(19) United States
(12) Patent Application Publication
(10) Pub. No.: US 2008/0085923 A1
(43) Pub. Date: Apr. 10, 2008

(54) Thrombin Receptor Antagonists Based On The Modified Tricyclic Unit Of Himbacine

(75) Inventors: Samuel Chackalamannil, Califon, NJ (US); Yuguang Wang, Monroe, NJ (US); Tiruvettipuram K. Thiruvengadam, Kendall Park, NJ (US); Ilia Zavialov, Princeton, NJ (US); Tao Li, Scotch Plains, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/866,012

(22) Filed: Oct. 2, 2007

Related U.S. Application Data
(60) Provisional application No. 60/849,284, filed on Oct. 4, 2006.

(51) Int. Cl. A61K 31/4525; A61P 25/00; A61P 35/00; A61P 9/00; C07D 405/08

(52) U.S. Cl. 514/337; 546/284.1

(57) ABSTRACT

Multiple stereoisomers of the heterocyclic-substituted tricyclics of the formula: 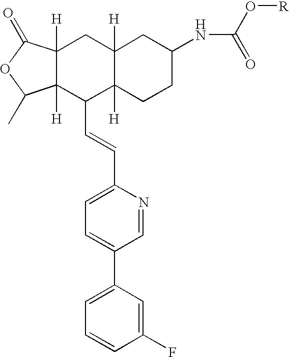 or a pharmaceutically acceptable salt, solvate, or ester of said compound wherein R and the stereochemistry are illustrated in the structural formulas herein are disclosed, as well as pharmaceutical compositions containing them and a method of treating diseases associated with thrombosis, atherosclerosis, restenosis, hypertension, angina pectoris, arrhythmia, heart failure, and cancer by administering said compounds. Combination therapy with other cardiovascular agents is also claimed.

This application claims the benefit of U.S. Provisional Application No. 60/849,284, filed Oct. 4, 2006, which is incorporated herein by reference in its entirety.

### BACKGROUND OF THE INVENTION

The present invention relates to himbacine derivatives, which can be useful as thrombin receptor antagonists in the treatment of diseases associated with thrombosis, atherosclerosis, restenosis, hypertension, angina pectoris, arrhythmia, heart failure, cerebral ischemia, stroke, neurodegenerative diseases and cancer. Thrombin receptor antagonists are also known as protease activated receptor-1 (PAR-1) antagonists. The compounds of the invention also can be useful as cannabinoid (CB₂) receptor inhibitors for the treatment of rheumatoid arthritis, systemic lupus erythematosus, multiple sclerosis, diabetes, osteoporosis, renal ischemia, cerebral stroke, cerebral ischemia, nephritis, inflammatory disorders of the lungs and gastrointestinal tract, and respiratory tract disorders such as reversible airway obstruction, chronic asthma and bronchitis. The invention also relates to pharmaceutical compositions comprising said compounds.

Thrombin is known to have a variety of activities in different cell types. Thrombin receptors are known to be present in such cell types as human platelets, vascular smooth muscle cells, endothelial cells and fibroblasts. It is therefore expected that thrombin receptor antagonists will be useful in the treatment of thrombotic, inflammatory, atherosclerotic and fibroproliferative disorders, as well as other disorders in which thrombin and its receptor play a pathological role.

Thrombin receptor antagonist peptides have been identified based on structure-activity studies involving substitutions of amino acids on thrombin receptors. In Bernatowicz et al., J. Med. Chem., 39 (1996) p. 4879-4887, tetra- and pentapeptides are disclosed as being potent thrombin receptor antagonists, for example N-trans-cinnamoyl-p-fluoroPhe-p-guanidinoPhe-Leu-Arg-NH₂ and N-trans-cinnamoyl-p-fluoroPhe-p-guanidinoPhe-Leu-Arg-Arg-NH₂. Peptide thrombin receptor antagonists are also disclosed in WO 94/03479, published Feb. 17, 1994. Properties of himbacine derived compounds that are thrombin receptor antagonists have been described. (Chackalamannil et. al. J. Med. Chem., 48 (2005), 5884-5887.)

Cannabinoid receptors belong to the superfamily of G-protein coupled receptors. They are classified into the predominantly neuronal CB₁ receptors and the predominantly peripheral CB₂ receptors. These receptors exert their biological actions by modulating adenylate cyclase and Ca⁺² and K⁺ currents. While the effects of CB₁ receptors are principally associated with the central nervous system, CB₂ receptors are believed to have peripheral effects related to bronchial constriction, immunomodulation and inflammation. As such, a selective CB₂ receptor binding agent is expected to have therapeutic utility in the control of diseases associated with rheumatoid arthritis, systemic lupus erythematosus, multiple sclerosis, diabetes, osteoporosis, renal ischemia, cerebral stroke, cerebral ischemia, nephritis, inflammatory disorders of the lungs and gastrointestinal tract, and respiratory tract disorders such as reversible airway obstruction, chronic asthma and bronchitis (R. G. Pertwee, Curr. Med. Chem. 6(8), (1999), 635; M. Bensaid, Molecular Pharmacology, 63 (4), (2003), 908.).

Himbacine, a piperidine alkaloid of the formula 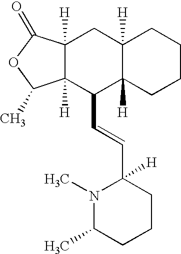
has been identified as a muscarinic receptor antagonist. The total synthesis of (+)-himbacine is disclosed in Chackalamannil et al., J. Am. Chem. Soc., 118 (1996), p. 9812-9813.

Substituted tricyclic thrombin receptor antagonists are disclosed in U.S. Pat. No. 6,063,847, U.S. Pat. No. 6,326,380, U.S. Pat. No. 6,645,987 (WO 01/96330), U.S. Ser. No. 10/271,715 and U.S. Ser. No. 10/412,982.

### SUMMARY OF THE INVENTION

The present invention provides compounds represented by the following formulas: 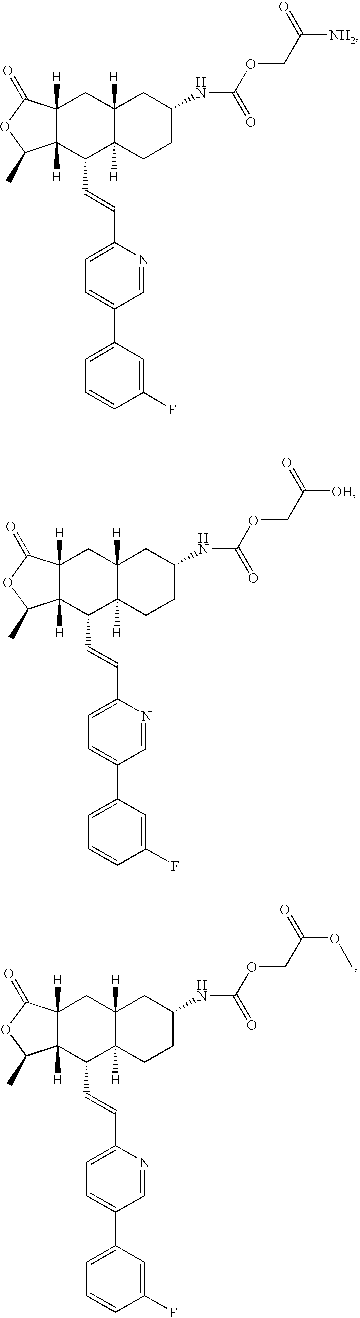 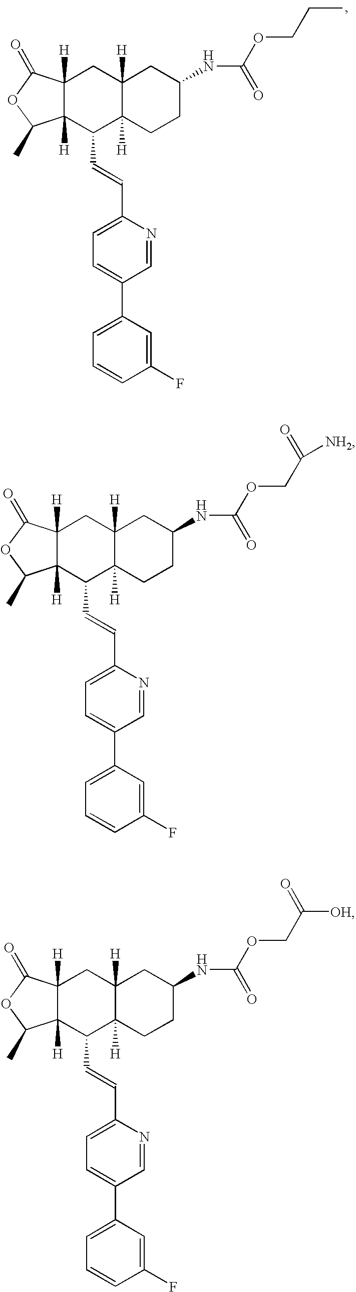 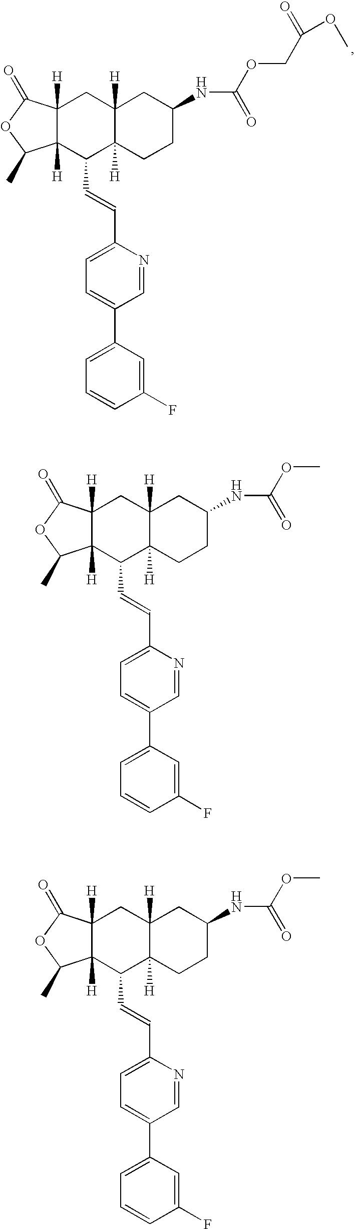 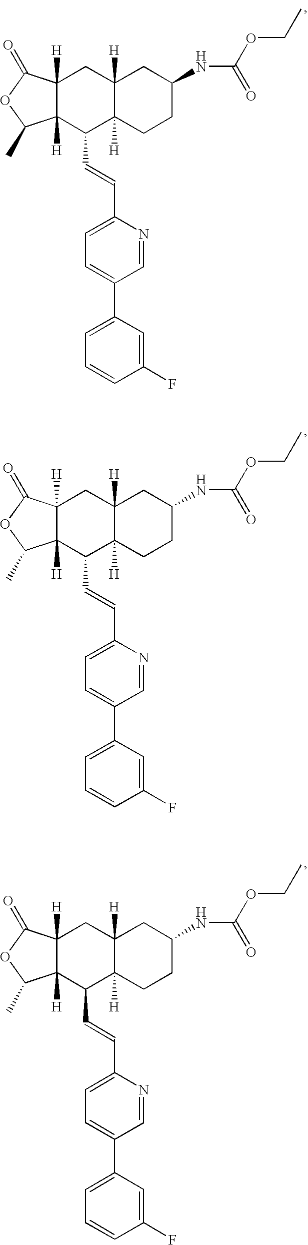 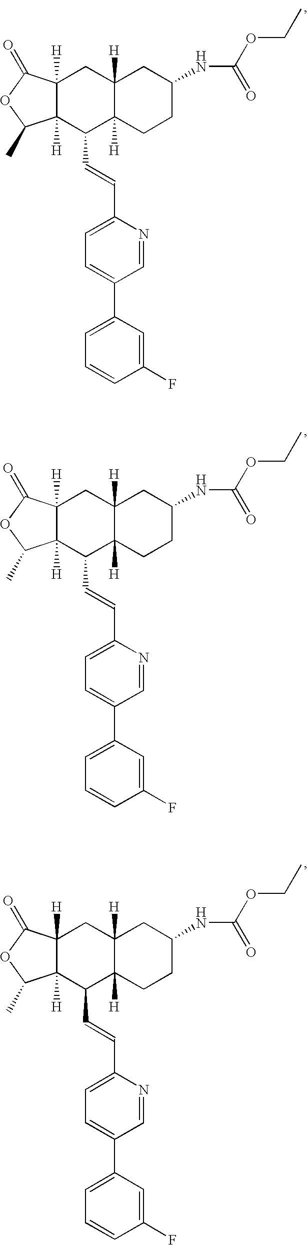 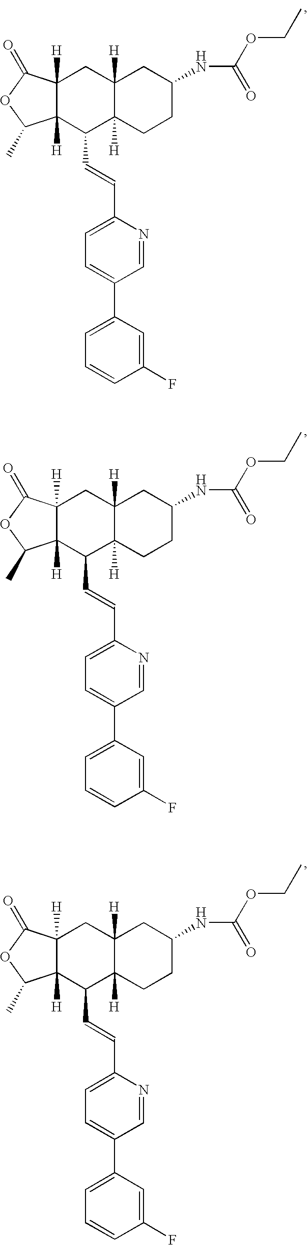 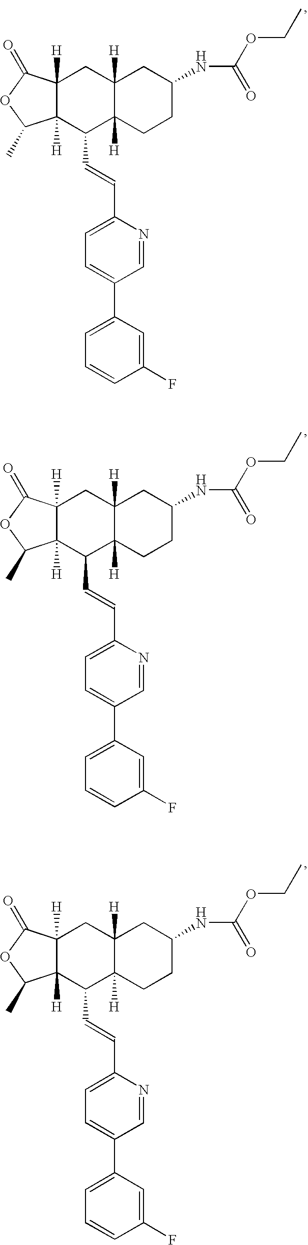 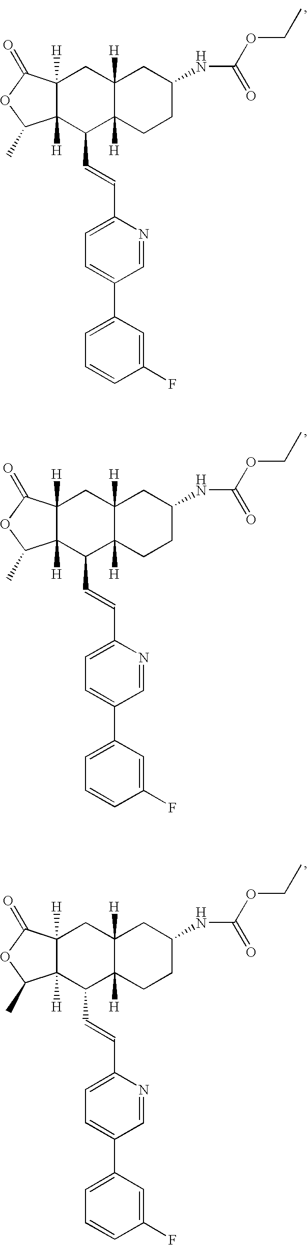 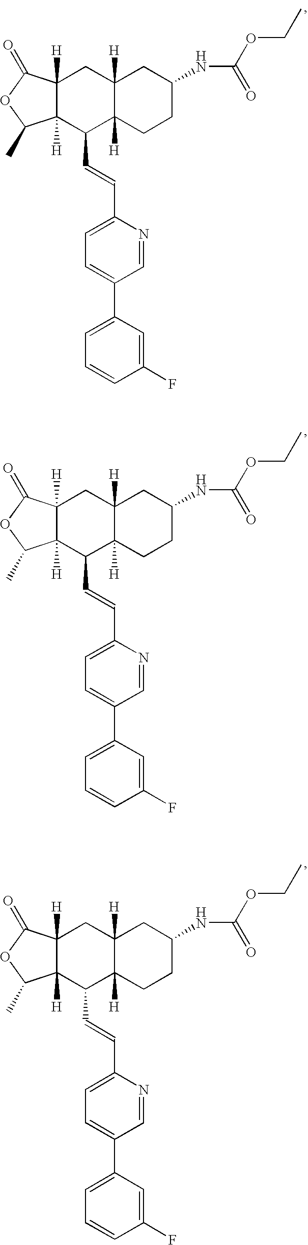 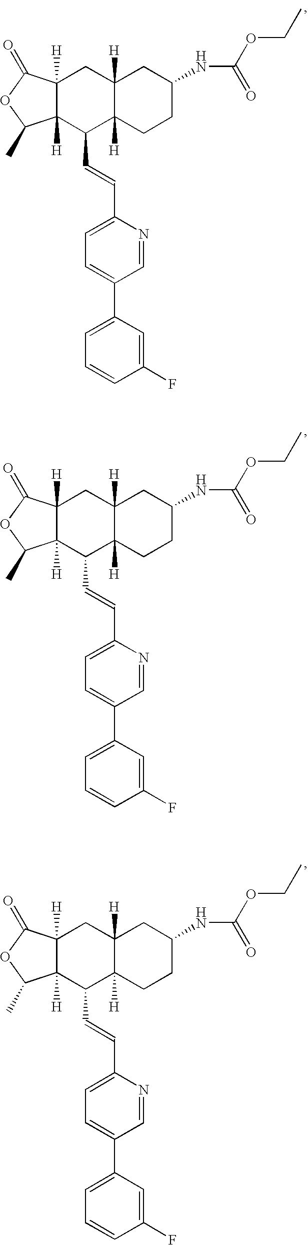 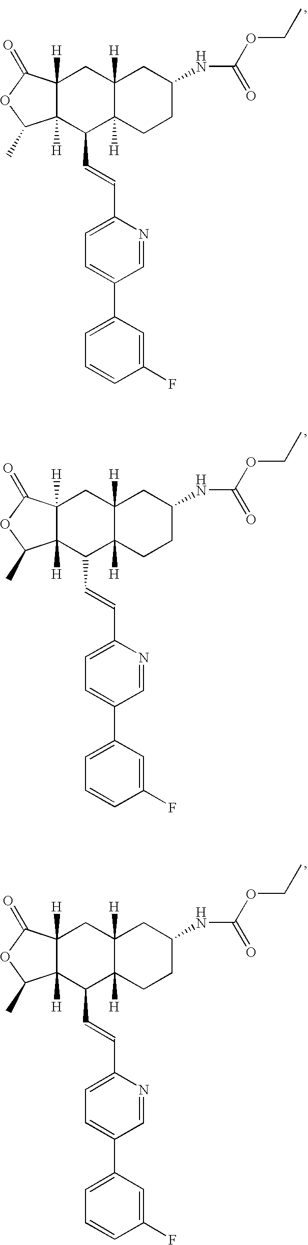 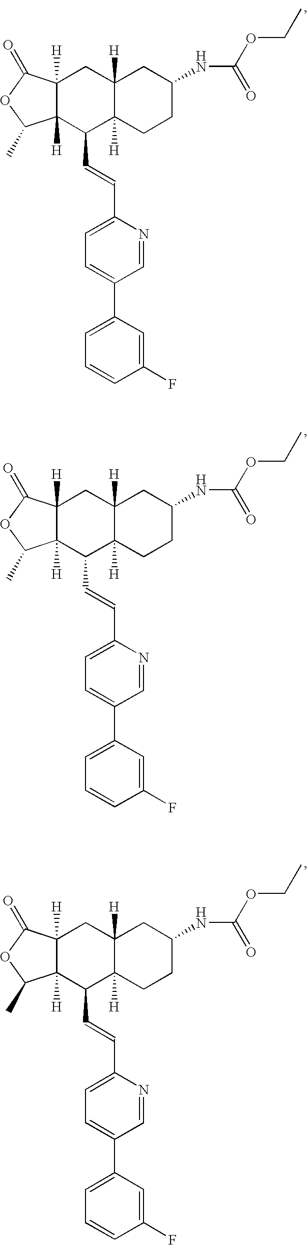 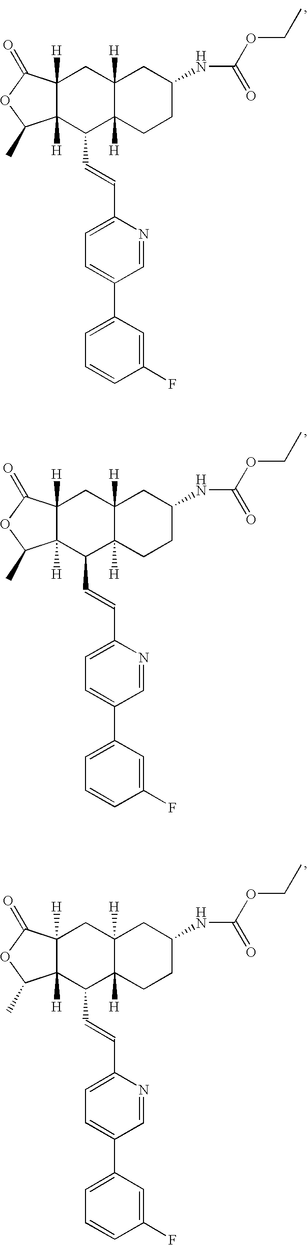 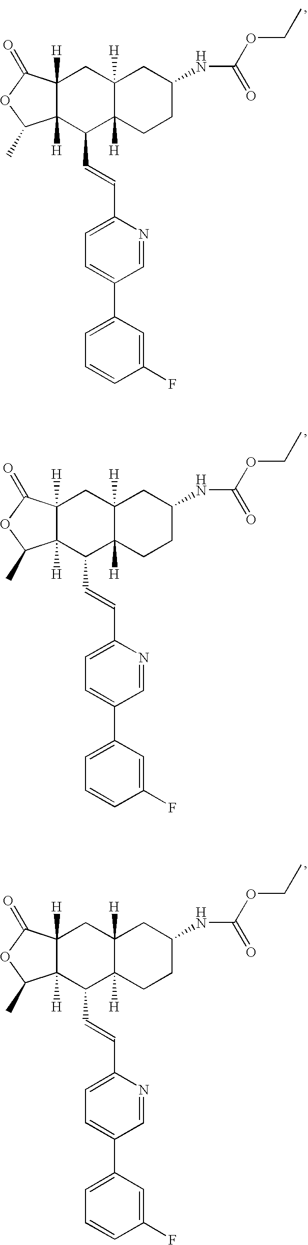 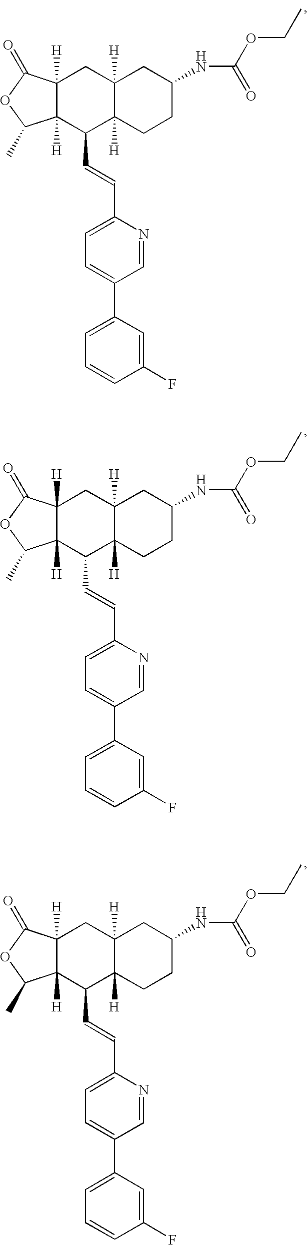 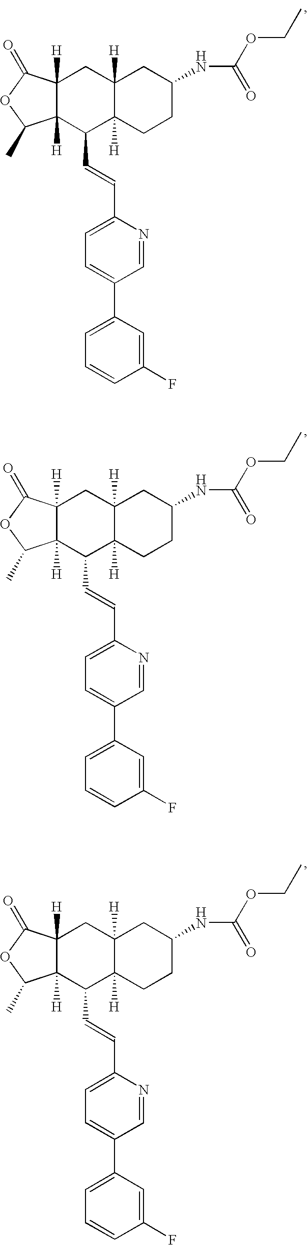 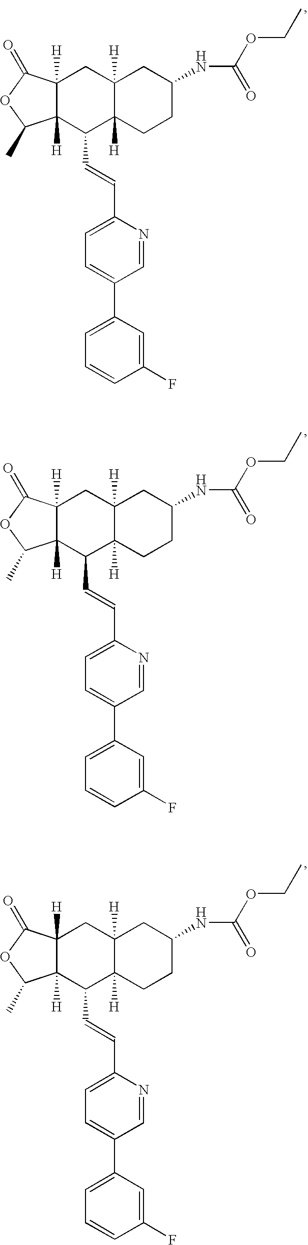 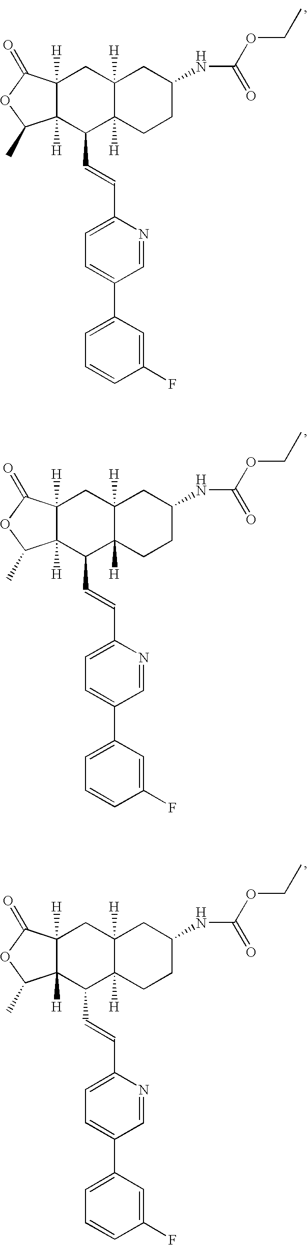 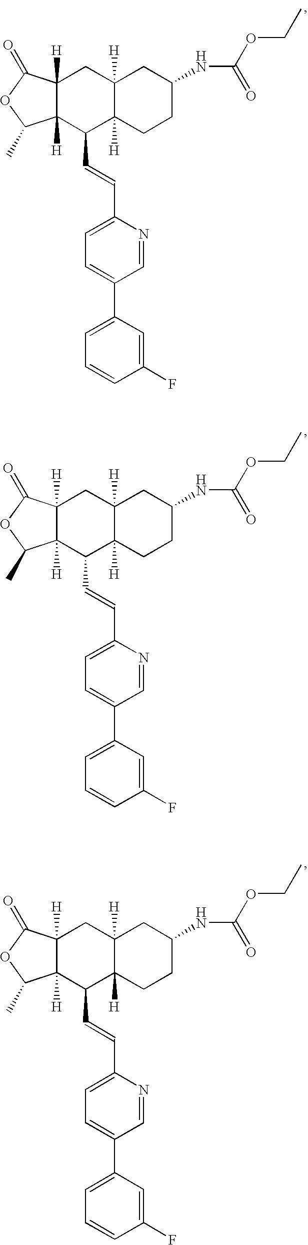 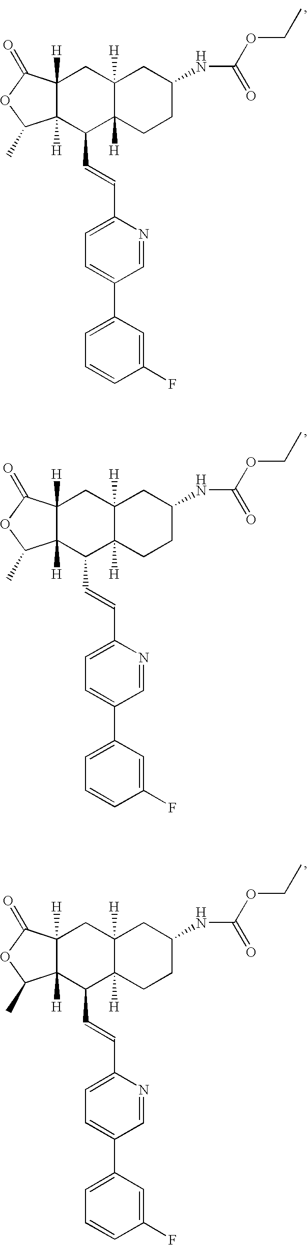 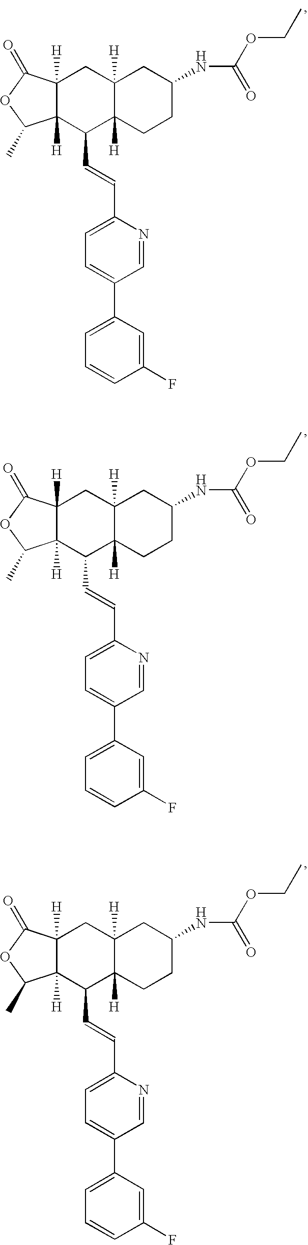 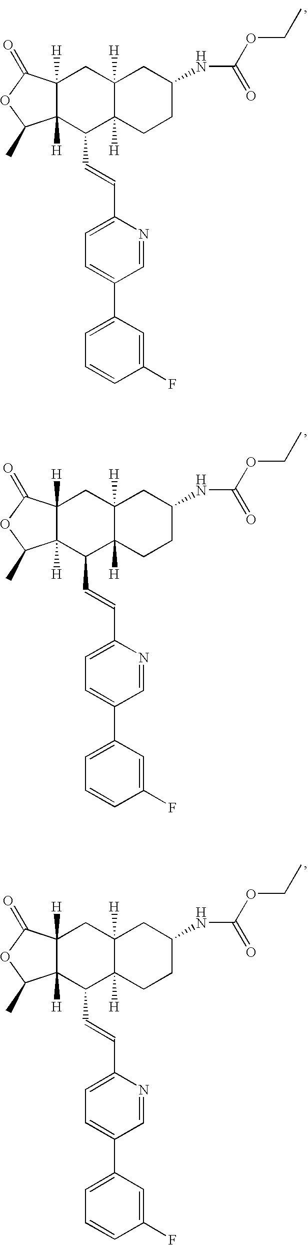 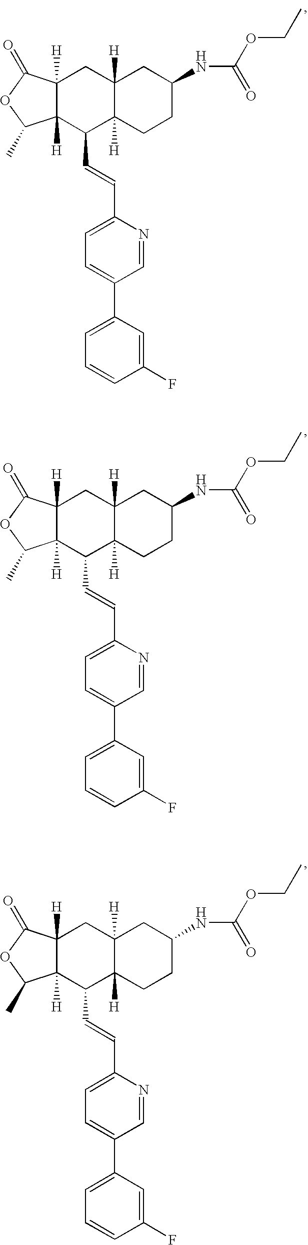 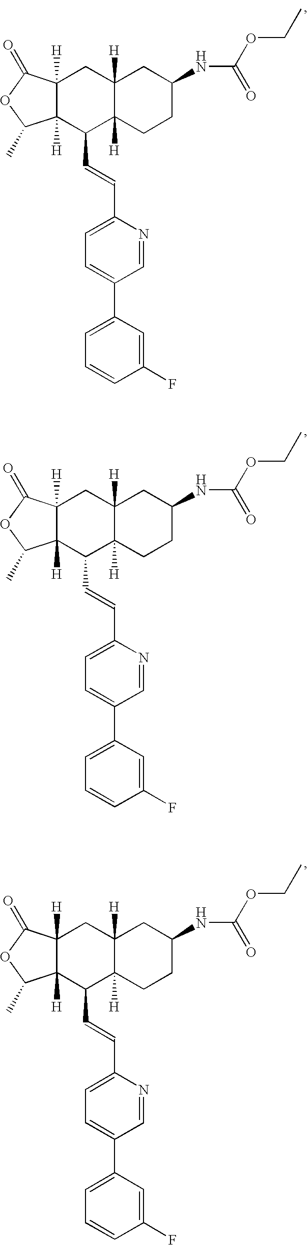 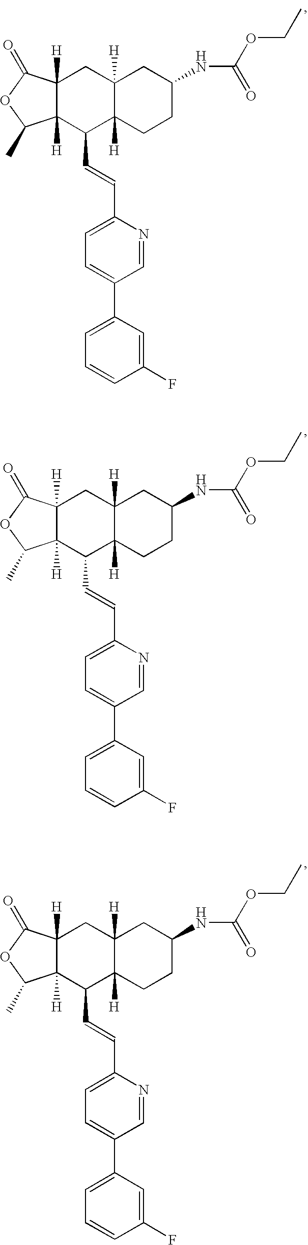 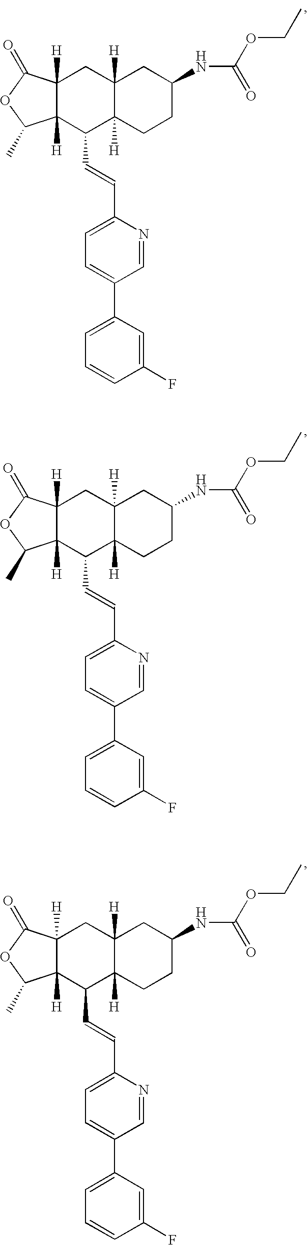 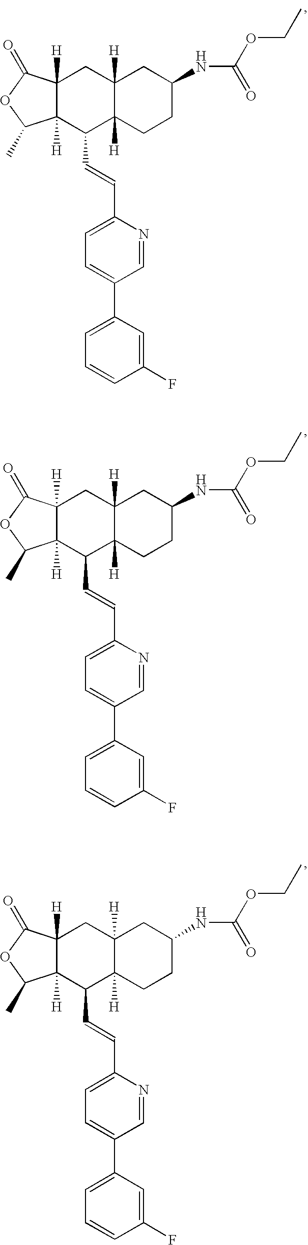 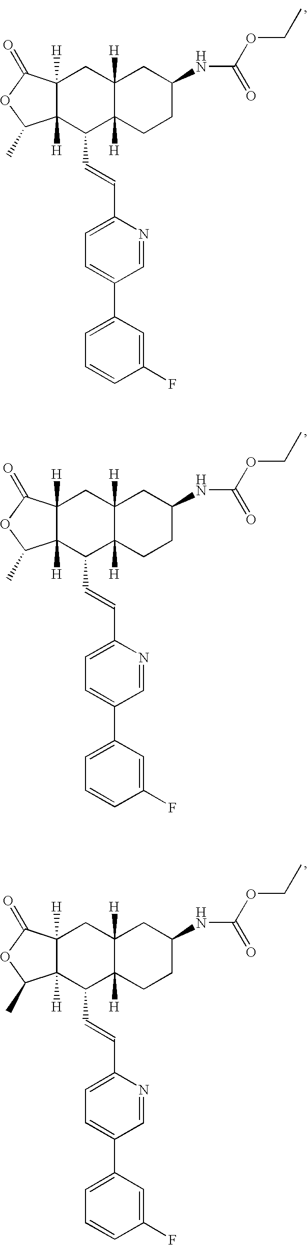 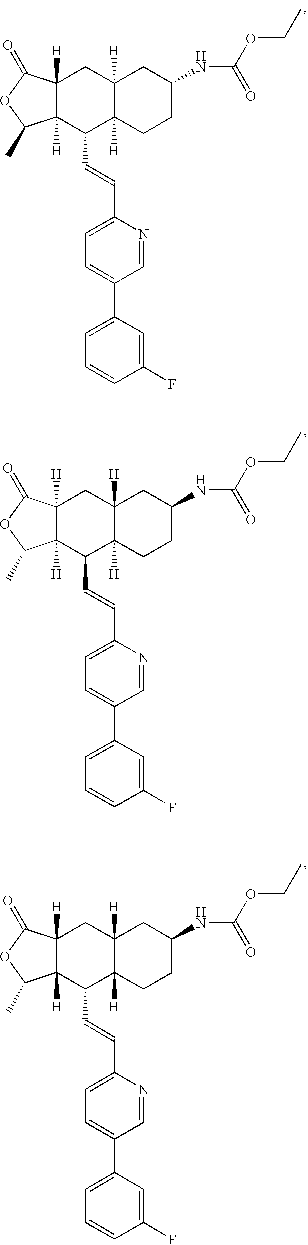 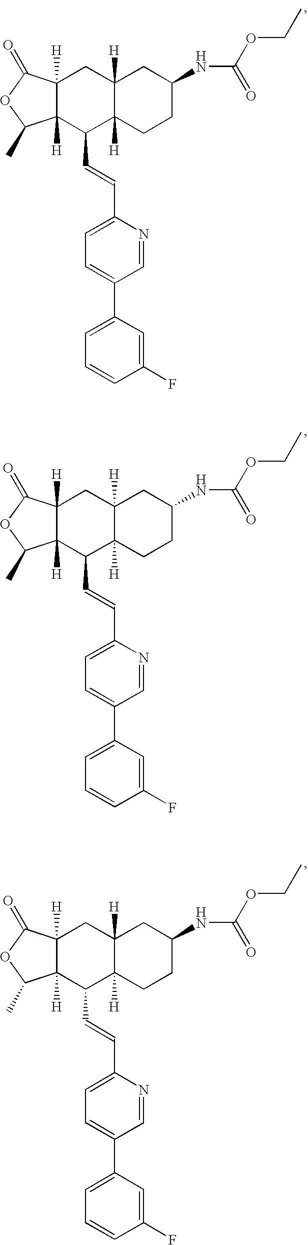 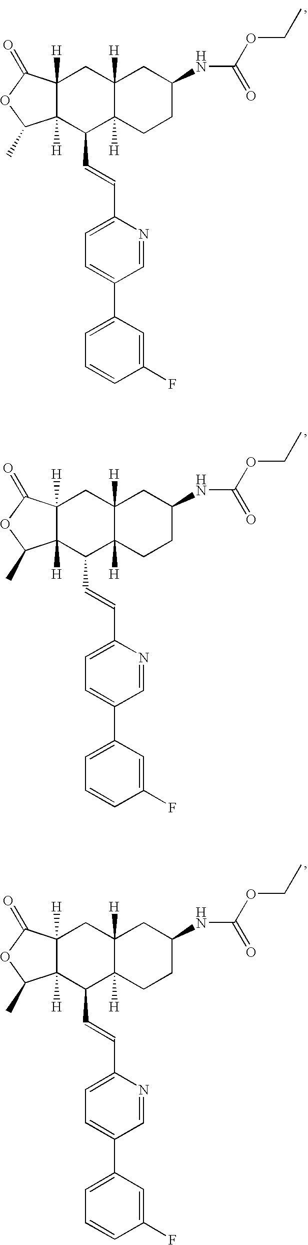 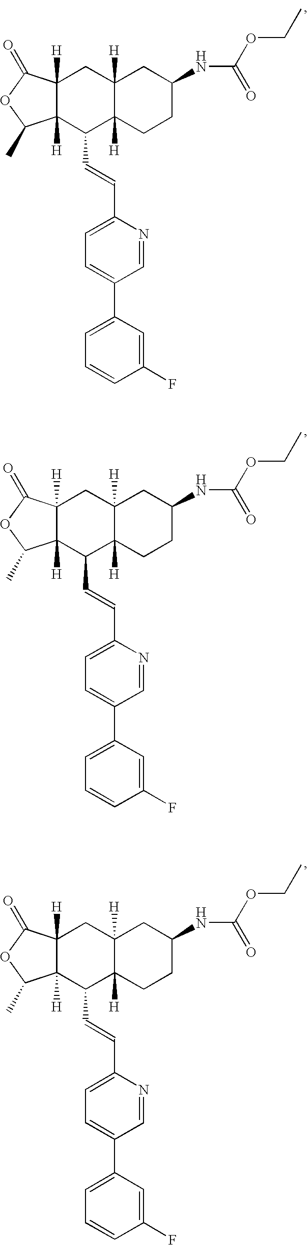 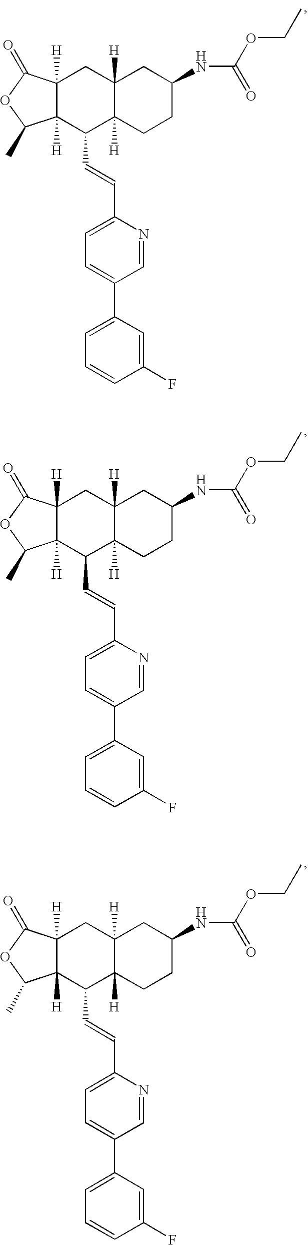 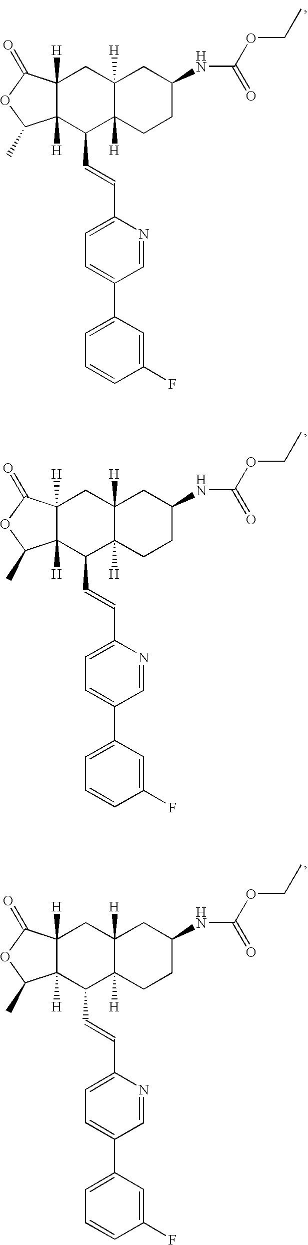 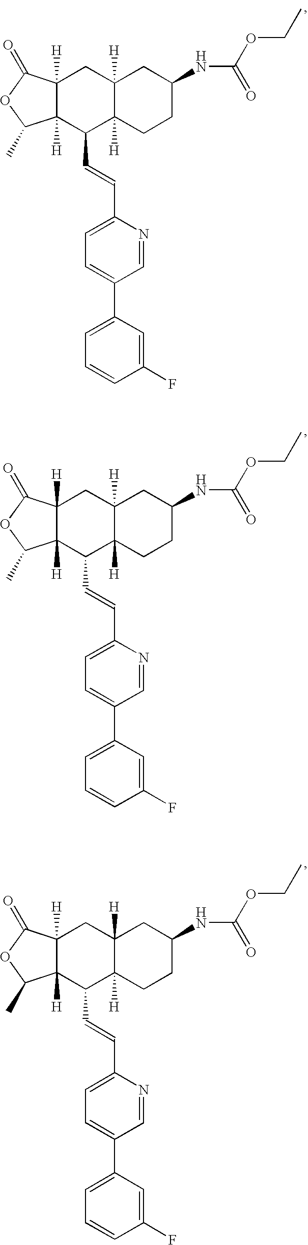 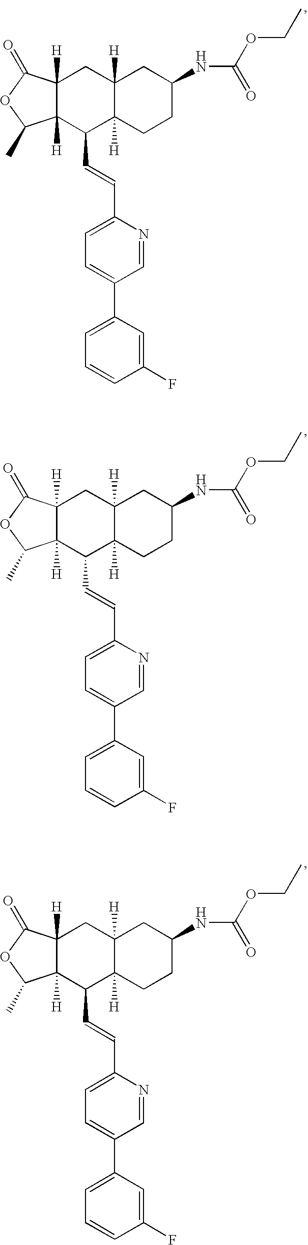 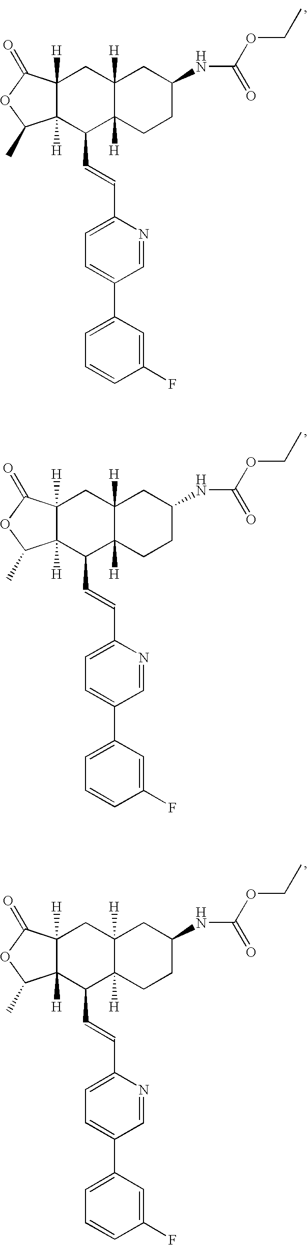 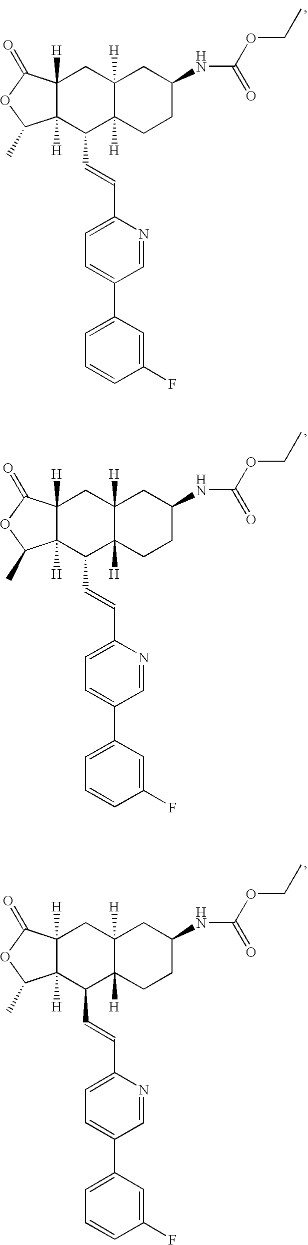 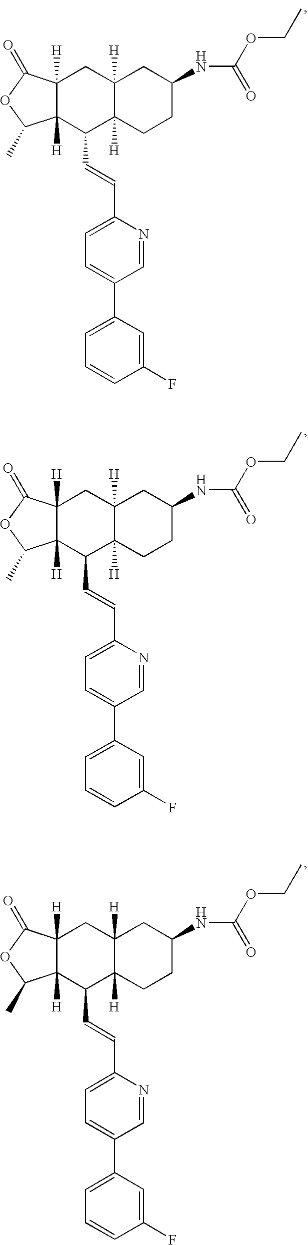 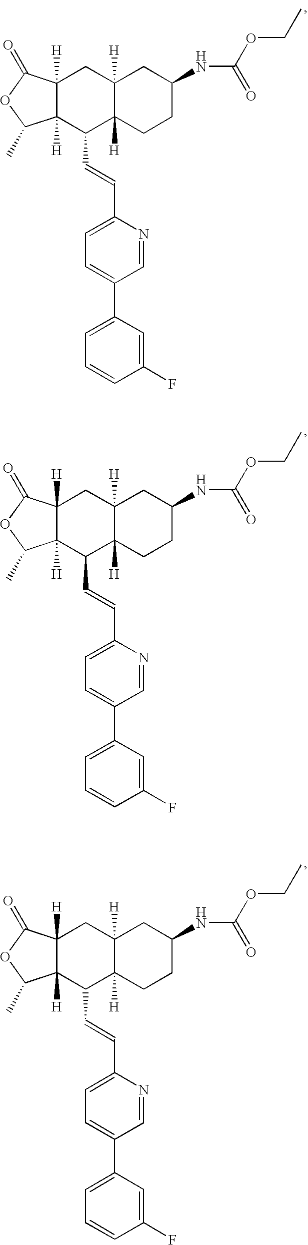 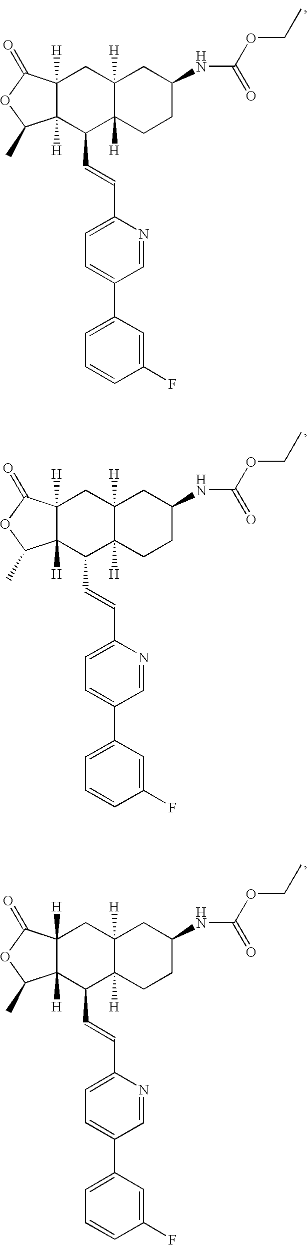 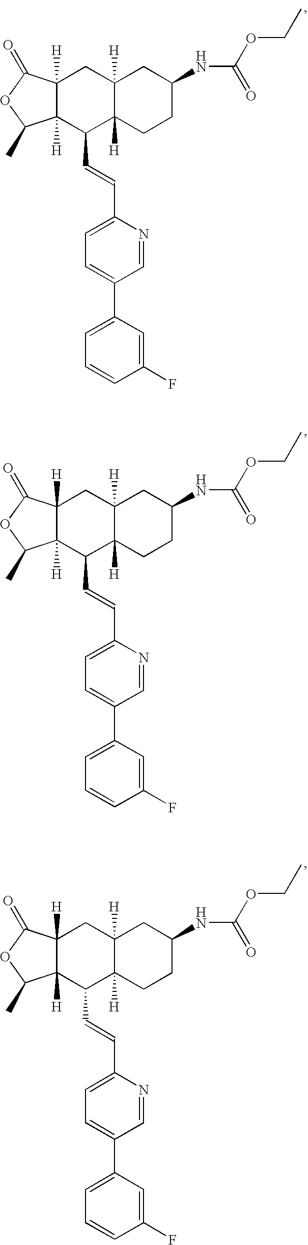 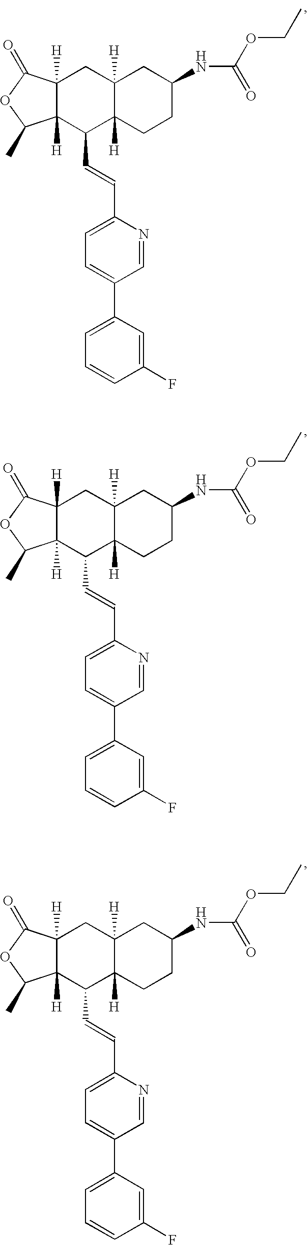 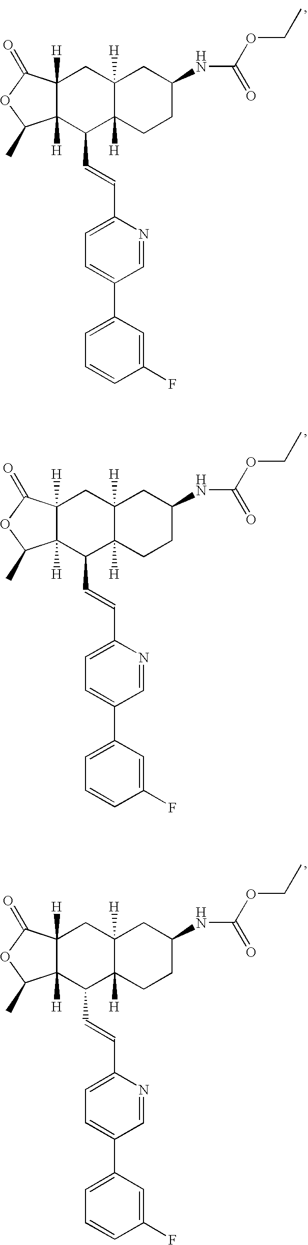 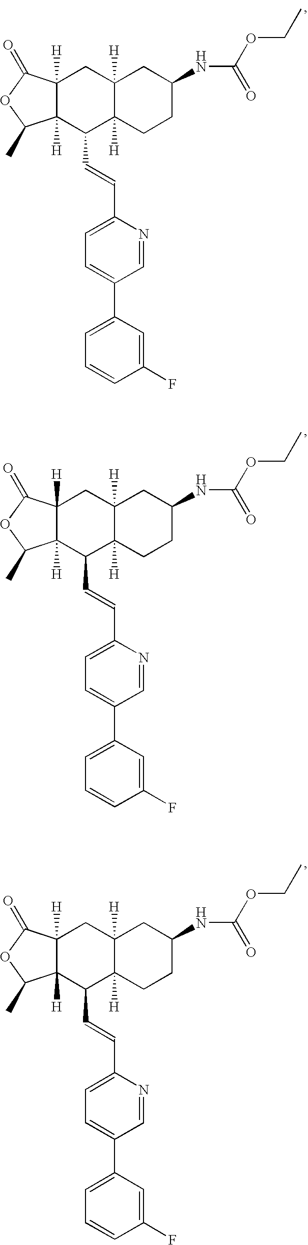 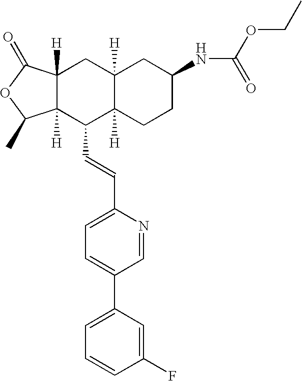
or a pharmaceutically acceptable salt, solvate, ester, polymorph, co-crystal, or polymers of any of said compounds.

Pharmaceutical compositions comprising at least one compound of the of the invention and at least one pharmaceutically acceptable carrier are also provided.

The compounds of the present invention can be useful as Thrombin receptor antagonists, also known as PAR-1 antagonists, or as cannabinoid (CB₂) receptor antagonists. Thrombin receptor antagonist compounds of the present invention can have anti-thrombotic, anti-platelet aggregation, anti-atherosclerotic, anti-restenotic anti-coagulant, and/or anti-inflammatory activity. CB₂ receptor inhibitor compounds of the present invention can be useful for the treatment of rheumatoid arthritis, systemic lupus erythematosus, multiple sclerosis, diabetes, osteoporosis, renal ischemia, cerebral stroke, cerebral ischemia, nephritis, inflammatory disorders of the lungs and gastrointestinal tract, and respiratory tract disorders such as reversible airway obstruction, chronic asthma and bronchitis.

Compounds of the invention can be useful for the treatment of thrombosis, atherosclerosis, restenosis, hypertension, angina pectoris, angiogenesis related disorders, arrhythmia, a cardiovascular or circulatory disease or condition, heart failure, acute coronary syndrome (ACS), myocardial infarction, glomerulonephritis, thrombotic stroke, thromboembolytic stroke, peripheral vascular diseases, deep vein thrombosis, venous thromboembolism, a cardiovascular disease associated with hormone replacement therapy, disseminated intravascular coagulation syndrome, cerebral infarction, migraine, erectile dysfunction, rheumatoid arthritis, rheumatism, astrogliosis, a fibrotic disorder of the liver, kidney, lung or intestinal tract, systemic lupus erythematosus, multiple sclerosis, osteoporosis, renal disease, acute renal failure, chronic renal failure, renal vascular homeostasis, renal ischemia, bladder inflammation, diabetes, diabetic neuropathy, cerebral stroke, cerebral ischemia, nephritis, cancer, melanoma, renal cell carcinoma, neuropathy, malignant tumors, neurodegenerative and/or neurotoxic diseases, conditions or injuries, Alzheimer's disease, an inflammatory disease or condition, asthma, glaucoma, macular degeneration, psoriasis, endothelial dysfunction disorders of the liver, kidney or lung, inflammatory disorders of the lungs and gastrointestinal tract, respiratory tract disease or condition, radiation fibrosis, endothelial dysfunction, periodontal diseases or wounds, or a spinal cord injury, or a symptom or result thereof, as well as other disorders in which thrombin and its receptor play a pathological role.

In particular, compounds of the present invention are used to treat acute coronary syndrome, myocardial infarction or thrombotic stroke.

Compounds of the present invention can also be used in a method to treat or prevent a condition associated with cardiopulmonary bypass surgery (CPB) comprising administering an effective amount of at least one thrombin receptor antagonist to a subject of said surgery. CPB surgery includes coronary artery bypass surgery (CABG), cardiac valvular repair and replacement surgery, pericardial and aortic repair surgeries. In particular, the present invention relates to a method of treating or preventing a condition associated with CABG surgery comprising administering an effective amount of at least one thrombin receptor antagonist to a subject of said surgery. The conditions associated with CABG are selected from the group consisting of: bleeding; thrombotic vascular events such as thrombosis, restenosis; vein graft failure; artery graft failure, atherosclerosis, angina pectoris; myocardial ischemia; acute coronary syndrome myocardial infarction; heart failure; arrhythmia; hypertension, transient ischemic attack, cerebral function impairment; thromboembolic stroke; cerebral ischemia; cerebral infarction; thrombophlebitis; deep vein thrombosis; and, peripheral vascular disease.

In another embodiment, compounds of the present invention can be useful in a method for treating and/or preventing radiation- and/or chemical-induced toxicity in non-malignant tissue in a patient comprising administering a therapeutically effective amount of at least one compound of the invention. In particular, the radiation- and/or chemical-induced toxicity is one or more of intestinal fibrosis, pneumonitis, and mucositis. In a preferred embodiment, the radiation- and/or chemical-induced toxicity is intestinal fibrosis. In another preferred embodiment, the radiation- and/or chemical-induced toxicity is oral mucositis. In yet another embodiment, the radiation- and/or chemical-induced toxicity is intestinal mucositis, intestinal fibrosis, intestinal radiation syndrome, or pathophysiological manifestations of intestinal radiation exposure.

The present invention also provides methods for reducing structural radiation injury in a patient that will be exposed, is concurrently exposed, or was exposed to radiation and/or chemical toxicity, comprising administering a therapeutically effective amount of at least one compound of the invention. The present invention also provides methods for reducing inflammation in a patient that will be exposed, is concurrently exposed, or was exposed to radiation and/or chemical toxicity, comprising administering a therapeutically effective amount of at least one compound of the invention. The present invention also provides methods for adverse tissue remodeling in a patient that will be exposed, is concurrently exposed, or was exposed to radiation and/or chemical toxicity, comprising administering a therapeutically effective amount of at least one compound of the invention. The present invention also provides methods for reducing fibroproliferative tissue effects in a patient that will be exposed, is concurrently exposed, or was exposed to radiation and/or chemical toxicity, comprising administering a therapeutically effective amount of at least one compound of the invention.

The present invention further provides methods useful for treating a cell proliferative disorder in a patient suffering therefrom comprising administering a therapeutically effective amount of at least one compound of the invention. In one embodiment, the cell proliferative disorder is pancreatic cancer, glioma, ovarian cancer, colorectal and/or colon cancer, breast cancer, prostate cancer, thyroid cancer, lung cancer, melanoma, or stomach cancer. In one embodiment, the glioma is an anaplastic astrocytoma. In another embodiment, the glioma is a glioblastoma multiforme.

As used above, the term inflammatory disease or condition includes irritable bowel syndrome, Crohn's disease, nephritis or a radiation- or chemotherapy-induced proliferative or inflammatory disorder of the gastrointestinal tract, lung, urinary bladder, gastrointestinal tract or other organ. The term respiratory tract disease or condition includes reversible airway obstruction, asthma, chronic asthma, bronchitis or chronic airways disease. “Cancer” includes renal cell carcinoma or an angiogenesis related disorder. “Neurodegenerative disease” includes Parkinson's disease, amyotropic lateral sclerosis, Alzheimer's disease, Huntington's disease or Wilson's disease.

Certain embodiments of this invention also relate to a method of using an effective amount of at least one compound of the invention in combination with one or more additional agents for the treatment of thrombosis, atherosclerosis, restenosis, hypertension, angina pectoris, angiogenesis related disorders, arrhythmia, a cardiovascular or circulatory disease or condition, heart failure, acute coronary syndrome (ACS), myocardial infarction, glomerulonephritis, thrombotic stroke, thromboembolytic stroke, peripheral vascular diseases, deep vein thrombosis, venous thromboembolism, a cardiovascular disease associated with hormone replacement therapy, disseminated intravascular coagulation syndrome, cerebral infarction, migraine, erectile dysfunction, rheumatoid arthritis, rheumatism, astrogliosis, a fibrotic disorder of the liver, kidney, lung or intestinal tract, systemic lupus erythematosus, multiple sclerosis, osteoporosis, renal disease, acute renal failure, chronic renal failure, renal vascular homeostasis, renal ischemia, bladder inflammation, diabetes, diabetic neuropathy, cerebral stroke, cerebral ischemia, nephritis, cancer, melanoma, renal cell carcinoma, neuropathy, malignant tumors, neurodegenerative and/or neurotoxic diseases, conditions or injuries, Alzheimer's disease, an inflammatory disease or condition, asthma, glaucoma, macular degeneration, psoriasis, endothelial dysfunction disorders of the liver, kidney or lung, inflammatory disorders of the lungs and gastrointestinal tract, respiratory tract disease or condition, radiation fibrosis, endothelial dysfunction, periodontal diseases or wounds, or a spinal cord injury, or a symptom or result thereof. It is contemplated that a combination of this invention may be useful in treating more than one of the diseases listed.

For treating and/or preventing radiation- and/or chemical-induced toxicity in non-malignant tissue, the present invention includes administering to a patient in need of such treatment an effective amount of a combination of at least one compound of the invention and one or more radiation-response modifiers selected from the group consisting of Kepivance™ (palifermin), L-glutamine, teduglutide, sucralfate mouth rinses, iseganan, lactoferrin, mesna and trefoil factor.

For treating a cell proliferative disorder the present invention includes administering to a patient in need of such treatment an effective amount of a combination of at least one compound of the invention and another antineoplastic agent. In one embodiment, the other antineoplastic agent is temozolomide and the cell proliferative disorder is glioma. In another embodiment, the other antineoplastic agent is interferon and the cell proliferative disorder is melanoma. In one embodiment, the other antineoplastic agent is PEG-Intron (peginterferon alpha-2b) and the cell proliferative disorder is melanoma.

Pharmaceutical compositions comprising a therapeutically effective amount of a combination of at least one compound of the invention and at least one additional cardiovascular agent in a pharmaceutically acceptable carrier are also provided.

Pharmaceutical compositions comprising a therapeutically effective amount of a combination of at least one compound of the invention and a radiation-response modifier in a pharmaceutically acceptable carrier are also provided.

Pharmaceutical compositions comprising a therapeutically effective amount of at least one compound of the invention and an antineoplastic agent in a pharmaceutically acceptable carrier are also provided.

It is further contemplated that the combination of the invention can be provided as a kit comprising in a single package of at least one compound of the invention in a pharmaceutical composition, and at least one separate pharmaceutical composition comprising a cardiovascular agent.

### DETAILED DESCRIPTION

In one embodiment, the present invention discloses compounds represented by the above listed structural formulas, or pharmaceutically acceptable salt, solvate, ester, polymorph, co-crystal, or polymers thereof.

As used above, and throughout this disclosure, the terms, unless otherwise indicated, shall be understood to have the meanings as defined in US Pub. No. 2003/0216437 A1 (pg 4, paragraph 0069 to pg 6, paragraph 0098).

The compounds of this invention may contain asymmetric or chiral centers, and, therefore, exist in different stereoisomeric forms. It is intended that all stereoisomeric forms of the compounds of this invention as well as mixtures thereof, including racemic mixtures, form part of the present invention. In addition, the present invention embraces all geometric and positional isomers. For example, if a compound of this invention incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of this invention may be atropisomers (e.g., substituted biaryls) and are considered as part of this invention. Enantiomers can also be separated by use of chiral HPLC column.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of this invention incorporates a double bond or a fused ring, both the cis- and trans-forms, as well as mixtures, are embraced within the scope of the invention. Also, for example, all keto-enol and imine-enamine forms of the compounds are included in the invention.).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

Polymorphic forms of the compounds of this invention, and of the salts, solvates, esters and prodrugs of the compounds of this invention, are intended to be included in the present invention.

The compounds according to the invention have pharmacological properties; in particular, the compounds of this invention can be nor-seco himbacine derivatives useful as thrombin receptor antagonists.

Compounds of the invention have at least one asymmetrical carbon atom and therefore all isomers, including enantiomers, stereoisomers, rotamers, tautomers and racemates of the compounds of this invention (where they exist) are contemplated as being part of this invention. The invention includes d and l isomers in both pure form and in admixture, including racemic mixtures. Isomers can be prepared using conventional techniques, either by reacting optically pure or optically enriched starting materials or by separating isomers of a compound of this invention. Isomers may also include geometric isomers, e.g., when a double bond is present. Polymorphous forms of the compounds of this invention, whether crystalline or amorphous, also are contemplated as being part of this invention.

The compounds according to the invention have pharmacological properties; in particular, the compounds of the invention can be nor-seco himbacine derivatives useful as thrombin receptor antagonists.

Compounds of the invention have at least one asymmetrical carbon atom and therefore all isomers, including enantiomers, stereoisomers, rotamers, tautomers and racemates of the compounds of the invention (where they exist) are contemplated as being part of this invention. The invention includes d and l isomers in both pure form and in admixture, including racemic mixtures. Isomers can be prepared using conventional techniques, either by reacting optically pure or optically enriched starting materials or by separating isomers of a compound of the invention. Isomers may also include geometric isomers, e.g., when a double bond is present. Polymorphous forms of the compounds of the invention, whether crystalline or amorphous, also are contemplated as being part of this invention.

Another embodiment of the invention discloses a method of making the compounds disclosed herein. The intermediates can be obtained by the methods disclosed in any of U.S. Pat. No. 6,063,847, U.S. Pat. No. 6,326,380, U.S. Pat. No. 6,645,987 and U.S. Ser. No. 10/271,715, all of which are incorporated herein by reference. The compounds may be prepared by several techniques known in the art, typical procedures are shown in Schemes 1 to 3 below.

The illustrations should not be construed to limit the scope of the invention, which is defined in the appended claims. Alternative mechanistic pathways and analogous structures will be apparent to those skilled in the art.

In the procedures, the following abbreviations are used:

DABCO: 1,4-diazabicyclo(2,2,2)octane

DBU: 1,8-Diazabicyclo[5.4.0]undec-7-ene

DCC: Dicyclohexylcarbodiimide

DCM: Dichloromethane

DMAP: 4-Dimethyl aminopyridine

DMF: N,N-Dimethylformamide

HPLC: High Performance Liquid Chromatography

LAH: Lithium aluminum hydride

LDA: Lithium diisopropylamide

MTBE: Methyl tertiary butyl ether

PhSeCl: phenylselenyl chloride

TEA: Triethylamine

TFA: Trifluoroacetic acid

THF: Tetrahydrofuran

THP: Tetrahydropyran

### EXPERIMENTAL EXAMPLES

The syntheses of all stereoisomers contemplated in this invention can be carried out either according to Scheme 1, Scheme 2, or Scheme 3.

### Scheme 1

Scheme 1 outlines the synthesis of isomer 10. The necessary precursor is resolved from racemic propargyl derivative 1 and further elaborated to the Diels-Alder precursor 4 as shown in scheme 1. The general approach involves a key intramolecular Diels-Alder reaction of intermediate 4 to form the tricyclic amide 6. The amide 6 was hydrolyzed to form the carboxylic acid 7 which was converted to the aldehyde 8, via the corresponding acid chloride. Emmons-Wadsworth reaction of aldehyde 8 with the phosphonate 9 yielded the desired target 10.
Preparation of: 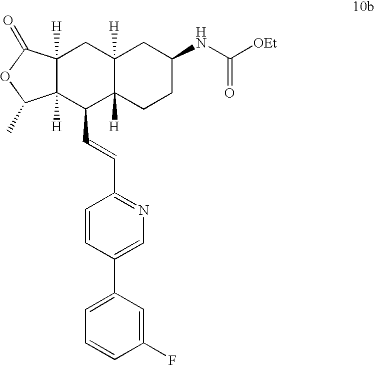
Step 1:

### Preparation of (S)-1 from Racemic (RS)-1

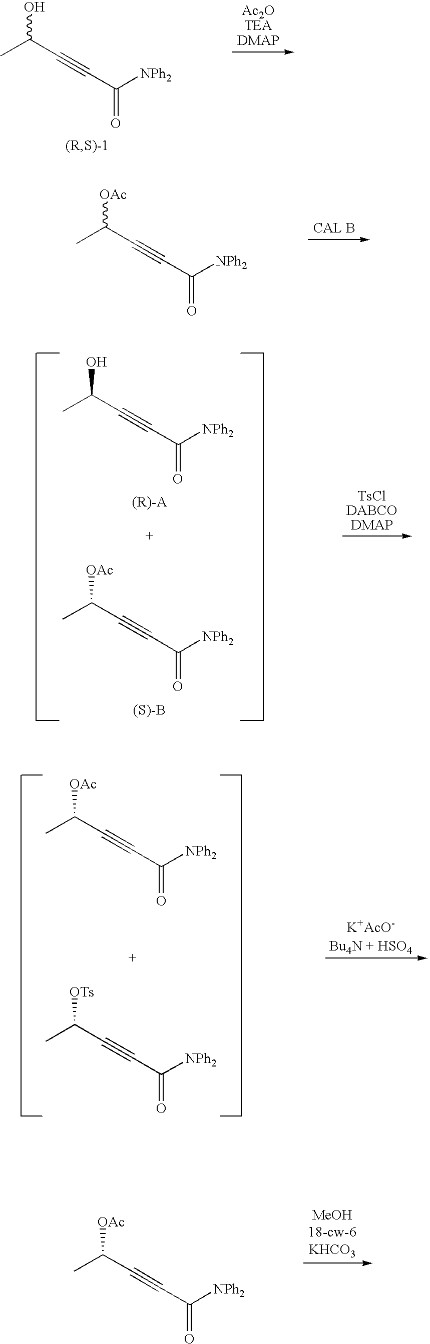

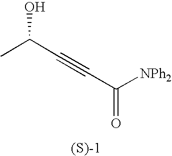

Methyl tertiary butyl ether (MTBE) (300 ml), (RS)-1 (50 g), triethylamine (TEA) (26.7 g), 4-(dimethylamine) pyridine (DMAP) (0.5 g) and acetic anhydride (28.9 g) were combined and agitated at 18° C. for 20 h. The reaction mixture was quenched with sulfuric acid (200 ml, 8%) and extracted. The organic phase was washed with a sodium bicarbonate solution (200 ml, 8%) and re-extracted. The solvent was removed from the organic phase by evaporation and the solution was reconstituted in 150 ml Toluene.

The toluene solution was mixed with 300 ml phosphate buffer (0.1 M) before adding 17 ml CAL B L. (Novozyme, Franklinton, N.C.). The hydrolysis reaction was carried out in a biphasic system. The pH of the aqueous phase was maintained at 7.0 by titration of 2 N NaOH with a pH stat. After 20 h, the conversion reached 51%, giving (R)-A and (S)—B in 97%, and 99% ee, respectively. The reaction mixture was filtered through a celite pad and the aqueous phase was removed.

The organic phase was concentrated to 100 ml by distillation and dry toluene (200 ml) was added. The reaction mixture was chilled to 0° C. then a solution of tosyl chloride in acetonitrile (21.5 g in 40 ml) was added. A solution of acetonitrile (60 ml) and 1,4-diazabicyclo(2,2,2)octane (DABCO) (13.7 g) and 4-(dimethylamino)pyridine (DMAP) (0.57 g) was added over 30 minutes at 0° C. After agitation for one more hour, the solution was quenched in sulfuric acid (200 ml 8%). The solution was extracted and the aqueous phase was removed and the organic phase was washed first with sodium bicarbonate (200 ml, 8%) then with brine (40 g of NaCl in 200 mL of water).

The inversion was carried out under phase transfer catalysis conditions. Water (4.8 ml) was added to the toluene solution. Potassium acetate (27.7 g), acetic acid (4 ml), and tetrabutylammonium acetate (6.4 g) were added to the toluene/water mixture. The reaction was agitated at 55° C. After 40 h, the conversion reached 94%, giving (S)—B as the only major product.

The toluene in the toluene/water mixture was replaced by methanol by adding 300 ml of methanol to the mixture, concentrating the mixture to 100 ml and repeating this process one time. Additional methanol (200 ml) was added for methanolysis and chilled to 5° C. Potassium bicarbonate (75 g) and 18-crown-6 (7.5 g) were added. The conversion from the (R) isomer to (S) isomer reached 98% after 10 h at 5° C. The solution was filtered through a celite pad after ethylacetate (100 ml) was added. Methanol was removed by distillation and the solution was reconstituted in ethylacetate (200 ml). The solution was washed first with sulfuric acid (200 ml, 8%), next with sodium bicarbonate (200 ml), and then with 200 ml brine.

The volume of the mixture was reduced to 150 ml by distillation. After heating to 70° C., heptane (450 ml) was added over 2 hours then the temperature was decreased to 20° C. to induce crystallization, Crystallization continued for 2 h and the crystals, S)-1 (31.7 g) were recovered by filtration, the purity was 98.2%, and ee was 99.5% for the S-enantiomer. (Mp 105° C., 1H NMR (400 MHz, DMSO-d₆) δ 1.04 (d, J=6.4 Hz, 3H), δ 4.27 (dq, J=5.6 Hz, 6.4 Hz, 1H), δ 5.49 (d, J=5.6 Hz, 1H), δ 7.2-7.5 (m, 10H); ¹³C NMR (DMSO-d₆) δ 23.7, 56.3, 76.9, 96.4, 126.8, 127.0, 128.5, 129.2, 129.4, 129.6, 141.5, 142.2, 152.9.)

Step 2:

### Preparation of 3 from (S)-1

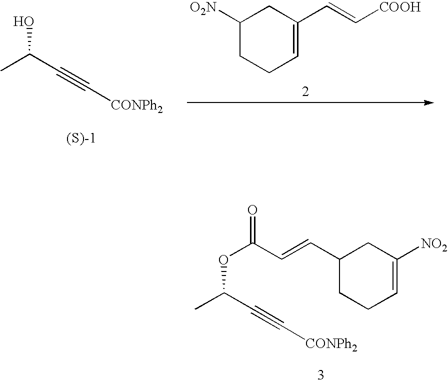

Compound 2 (90 g, 0.46 mole) was added to toluene (500 mL) and the suspension was cooled to about 0° C. N-methylmorpholine (91 mL, 0.83 mole) and trimethylacetyl chloride (56 mL, 0.46 mole) were slowly added while keeping the reaction temperature below 5° C. The reaction mixture was agitated for 1 hour at 0° C. and assayed for completion of formation of mixed anhydride (>90% complete). A solution of (S)-1 (100 g, 0.38 mole) in toluene (400 mL) and tetra hydro furan (220 mL) was added while keeping the reaction temperature below 5° C. This was followed by addition of a solution of 4-dimethylaminopyridine (5.5 g, 0.046 mole) in THF (45 mL). The mixture was agitated at about 0° C. for 8-12 hours until reaction completion (<0.2% (S)-23 remained). The reaction was quenched by adding a solution of 2.0 NH₂SO₄ (400 mL), warmed up to 25° C. and filtered through a pad of celite. The layers were separated and the organic layer was washed with 5% K₂CO₃ solution (3×300 mL) to remove excess 2 (<1% remained). The mixture was washed with 5% NaCl solution (300 mL), filtered through a pad of celite, and concentrated to about 500 mL final volume. Solution yield 90-95%. ¹H NMR (CDCl₃, 400 MHz) δ 7.05-7.35 (m, 11H), 6.13 (br, 1H), 5.62 (dd, J=16, 4 Hz, 1H), 5.31 (q, J=7 Hz, 1H), 4.67 (m, 1H), 2.62-2.78 (m, 2H), 2.58 (br, 2H), 2.05 (m, 2H), 1.22 (d, J=7 Hz, 3H).

Step 3:

### Preparation of Compound 4 from 3

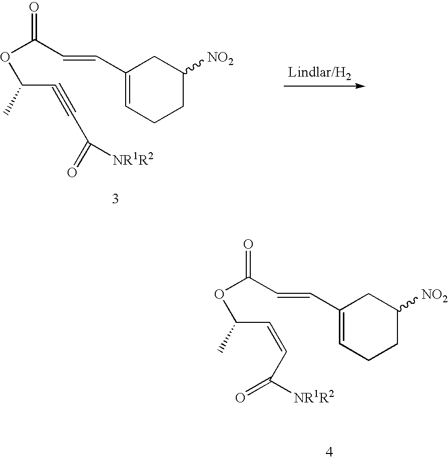

To a solution of 3 in toluene (50.0 g active, 112.5 mmol in 200 mL) Lindlar catalyst (2.5 g of 5% Pd/CaCO₃ with 5% Pb poisoned, 1.2 mmol) and quinoline (1.5 mL, 11.6 mmol) was added. The mixture was hydrogenated using 100 psi hydrogen at 25-30° C. until the reaction was completed as judged by HPLC. After removal of the catalyst by filtration, toluene was replaced with ethyl alcohol by regulated vacuum distillation of about 40° C. The product was dynamically crystallized from ethyl alcohol (180 mL) at 40° C. in the presence of triethyl amine (8.5 mL). The reaction mixture was slowly cooled to 5° C. over a period of 4 hours. After stirring at 5° C. for 3 hours, the product was filtered and washed with cold ethyl alcohol. The product was dried at 60° C. in a vacuum oven with nitrogen purge overnight to give 4 as a yellow crystalline solid. Yield: 73.7%. ¹H NMR (400 MHz, CDCl₃) δ 1.48 (d, J=6.4 Hz, 3H), 2.21-2.46 (m, 4H), 2.80 (m, 2H), 4.71 (m, 1H), 5.81-5.91 (m, 3H), 6.19 (m, 1H), 6.29 (q, J=6.4 Hz, 1H), 7.28-7.37 (m, 11H).

Step 4:

### Preparation of Compound 5 from Compound 4

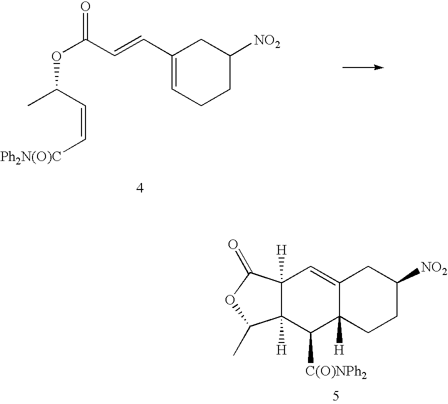

Compound 4 (25 g, 0.056 mol) and ethyl acetate (210 mL) were added into a 2 L 3 neck round bottom flask. The contents were stirred until compound 4 completely dissolved. The solution was washed with 0.25 M H₂SO₄ (75 mL) and water (3×75 mL). The organic phase was concentrated under reduced pressure to about 200 mL, and 1-methyl-2-pyrrolidinone (50 mL) was added. The solution was heated under distillation mode until a temperature of 145° C. was attained. The solution was held at this temperature for 3.5 h. The solution was cooled to room temperature, and 1,8-Diazabicyclo[5.4.0]undec-7-ene (DBU) (0.57 mL, 6.8 mol %) was added. The solution was stirred for 1 h and was quenched with 0.1 M H₂SO₄ (125 mL) and the product was extracted into ethyl acetate (125 mL). The organic phase was washed with water (125 mL) and was treated with DARCO-G60 (2.5 g) at 65° C. for 1 h. The suspension was filtered through a pad of Celite while the solution remained hot. The solution was concentrated by atmospheric distillation to 38 mL. The remaining ethyl acetate was replaced with isopropyl alcohol by azeotropic distillation. The volume of the solution was adjusted to 225 mL. The solution was diluted with ethyl alcohol and denatured with toluene (0.5%, 100 mL). The solution was slowly cooled to about 65° C. and DBU (0.29 mL, 3.4 mol %) was added. The suspension was slowly cooled to 15° C. and held at this temperature for 5 h. The product was filtered and washed with a 2:1 mixture of isopropyl alcohol and ethyl alcohol (50 mL), 19.3 g of compound 5 was obtained upon drying for 24 h at 50° C. (90.2 wt % purity, 17.4 g active, 72.5% yield). ¹H NMR (400 MHz, CDCl₃): δ 0.99 (m, 1H), 1.56 (d, J=6.0 Hz, 3H), 2.03 (m, 1H), 2.25-2.31 (m, 1H), 2.42-2.53 (m, 2H), 2.62-2.76 (m, 3H), 2.86-2.91 (m, 1H), 2.96-3.00 (m, 1H), 4.28-4.36 (m, 1H), 4.67-474 (m, 1H), 5.42 (br s, 1H), 7.22-7.53 (m, 10H).

Step 5:

### Preparation of Compound 6 from Compound 5

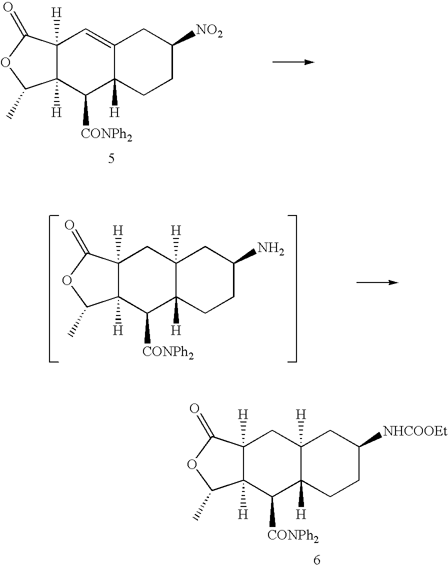

Compound 5(100 g), THF (600 ml), 10% palladium on carbon (50% wet, 35 g) and water (400 ml) were sequentially added to a three-neck flask equipped with an agitator, thermometer and nitrogen inlet. The mixture was agitated for about 10 minutes at room temperature and then heated to about 50° C. Formic acid (70 ml) was added slowly while the temperature was maintained between 45 and 55° C. The reaction mixture was agitated for 4 hours at 45-55° C. After the reaction was judged complete by HPLC, the reaction mixture was cooled to 20° C. and the pH was adjusted to 1-2 with 25% H₂SO₄ (60 mL). THE (200 mL) was added to the reaction mixture, which was then filtered through a pad of Celite to remove the catalyst. A mixed solution of THF (300 mL), water (300 ml) and H₂SO₄ (5 mL, 25%) was used to rinse the flask and catalyst, and filtered through the Celite. The combined solution was placed into a clean flask and the mixture was cooled to below 10° C. The pH was adjusted to about 9 with 25% NaOH (30 mL) at below 10° C. and NaCl (150 g) was then added. The mixture was warmed to 20° C. and two phases were separated. The aqueous phase was extracted with THF (400 mL) and the combined organic phases were washed with a brine solution (40 g of NaCl in 200 mL of water). The organic layer was cooled to 5° C. and triethyl amine (56 mL) was added. Then ethyl chloroformate (23.6 mL) was added slowly. The mixture was warmed to 20° C. and stirred for 30 minutes. After the reaction was judged complete, 200 ml of methyl tertiary butyl ether (MTBE) and 100 mL of water were added to the reaction mixture, followed by the slow addition of 100 mL of 25% H₂SO₄. The two phases were separated and the organic layer was washed with 200 ml of 12% H₂SO₄. The organic layer was then concentrated and azeotropically distilled with ethanol and water was at 70-80° C. The product was precipitated out from the ethanol-water solution with seeding at 55-65° C. After agitating for 1 hour at 55-65° C., 150 ml water was added at this temperature and held for 1 hour. After cooling to 15-25° C., the mixture was agitated for an additional 3 hours at 15-25° C. and then the product was filtered and washed with ethanol-water. The product, ent-6, was dried at 50-60° C. to provide an off-white solid (86 g, Yield, 85%). ¹H NMR (CDCl₃) δ 7.25-7.55 (m, 10H), 4.89 (m, 1H), 4.51 (bs, 1H), 4.09 (d, J=6.98 Hz, 2H), 3.49 (brs, 1H), 2.41 (m, 2H), 2.25 (m, 1H), 2.06 (d, J=10.8 Hz, 2H), 1.96 (d, J=10.9 Hz, 1H), 1.83 (ddd, J=13.5, 6.09, 2.51 Hz, 1H), 1.63 (m, 1H), 1.52 (d, J=58 Hz, 3H), 1.23 (m, 5H), 1.17 (q, J=11.5 Hz, 2H), 0.92 (q, J=11.5 Hz, 1H).

Step 6:

### Preparation of 8 from Ent-6

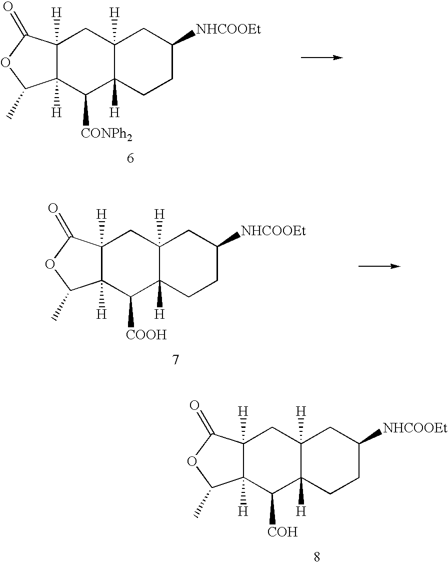

Compound 6 (10 g, 20.4 mmol) and tetrahydrofuran (THF) (50 mL) was added to a 250-mL 3-neck flask equipped with an agitator, thermometer, and a reflux condenser. To this solution was added an aqueous solution of sodium hydroxide (5% (w/w), 50 mL). The reaction mixture was then heated to and agitated at 40° C. for about 4 hours. When the hydrolysis reaction was judged complete, toluene (50 mL) was added and the mixture was agitated at a rather fast rate for about 10 minutes, The organic phase containing the by-product was separated from the aqueous phase containing product. The organic phase was back-extracted with 5% aqueous sodium hydroxide solution (50 mL). The combined aqueous solutions were extracted twice with toluene (2×50 mL) and the organic extracts were discarded. To the aqueous solution were added a solvent mixture of toluene (25 mL) and THF (50 mL). The resulting mixture was cooled to between 0 to 5° C. A 2 N hydrochloric acid aqueous solution (about 59 mL) was added to adjust the pH of the mixture from about 13 to 2.5 at 0 to 50° C. The aqueous phase was then separated from the organic phase and extracted with a solvent mixture of toluene (25 mL) and THF (50 mL). The organic phase and organic wash were combined and diluted with THF (50 mL). The mixture was then concentrated atmospherically to a final moisture content of ≦0.05% by repeated distillations, if necessary. The crude product 7 was used in the next step without further isolation and purification.

To a three-neck flask equipped with an agitator, thermometer and nitrogen inert were added the crude product 7 solution (containing about 3.1 g of active in 30 mL solution of THF) and anhydrous DMF (0.01 mL). After the mixture was agitated for 5 minutes, oxalyl chloride (1.22 mL) was added slowly while maintaining the batch temperature between 15 and 25° C. The reaction mixture was agitated for about an hour after the addition and checked by NMR for completion of reaction. After the reaction was judged complete, the mixture was concentrated under vacuum to 13.5 mL while maintaining the temperature of the reaction mixture below 30° C. The excess oxalyl chloride was removed completely by two cycles of vacuum concentration at below 50° C. with replenishment of toluene (31 mL) each time, resulting in a final volume of 7 mL. The reaction mixture was then cooled to 15 to 25° C., after which THF (16 mL) and 2,6-lutidine (2.2 mL) were added. The mixture was agitated for 16 hours at 20 to 25° C. under 100 psi hydrogen in the presence of dry 5% Pd/C (0.9 g). After the reaction was judged complete, the reaction mixture was filtered through celite to remove catalyst. More THF was added to rinse the hydrogenator and catalyst, and the reaction mixture was again filtered through celite. Combined filtrates were concentrated under vacuum at below 25° C. to 31 mL. MTBE (16 mL) and 10% aqueous solution of phosphoric acid (16 mL) were added for a thorough extraction at 10° C. to remove 2,6-lutidine. Then phosphoric acid was removed by extracting the organic layer with very dilute aqueous sodium bicarbonate solution (about 2%), which was followed by a washing with dilute brine (40 g of NaCl in 200 mL of water). The organic solution was concentrated to a volume of 9 mL for solvent replacement. Isopropyl alcohol (31 mL) was added to the concentrated crude product solution. The remaining residual solvent was purged to <0.5% of THF (by gas chromatography) by repeated concentration under vacuum to 7 mL, with replenishment of IPA (31 mL) before each concentration. The concentrated (7 mL) isopropyl alcohol solution was heated to 50° C., to initiate crystallization. To this mixture n-heptane (7 mL) was added very slowly while maintaining the batch temperature at 50° C. The crystallizing mixture was cooled very slowly over 2.5 hours to 25° C. Additional n-heptane (3.4 mL) was added very slowly into the suspension mixture at 25° C. The mixture was further cooled to 20° C. for about 20 hours. The solid was filtered and washed with a solvent mixture of 25% IPA in n-heptane, and then dried to provide 1.95 g of compound 8, which was a beige colored solid. (Yield-66%), ¹H NMR (CD3CN) 69.74 (d, J=3.03 Hz, 1H), 5.42 (br, 1H), 4.69 (m, 1H), 4.03 (q, J=7.02 Hz, 2H), 3.43 (qt, J=3.80, 7.84 Hz, 1H), 2.67 (m, 2H), 2.50 (dt, J=3.00, 8.52 Hz, 1H), 1.93 (d, J=12.0 Hz, 2H), 1.82 (dt, J=3.28, 9.75 Hz, 2H), 1.54 (qd, J=3.00, 10.5 Hz, 1H), 1.27 (d, J=5.97 Hz, 3H), 1.20 (m, 6H), 1.03-0.92 (m, 2H).

Step 7:

### Preparation of 10 from 8

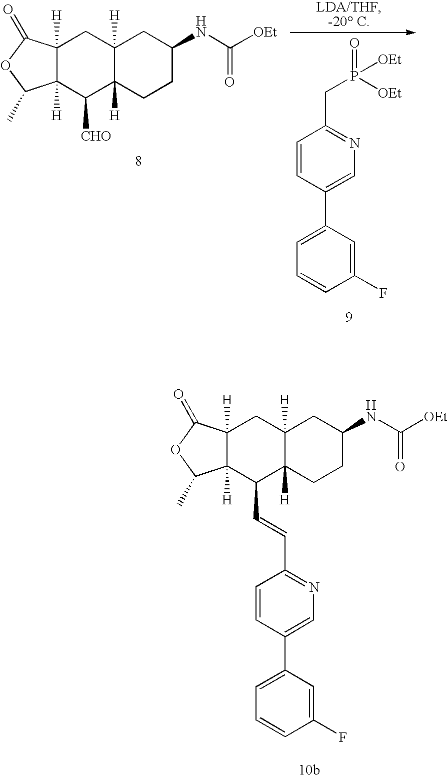

To a three-neck flask equipped with an agitator, thermometer and nitrogen inertion was added compound 9 (13.0 g) and THF (30 mL). The mixture was cooled to below −20° C. after which lithium diisopropylamide (2M, 20 mL) was slowly added. The reaction mixture was agitated for an additional hour (Solution A). To another flask was added compound 8 (10.0 g) and THF (75 mL). The mixture was stirred for about 30 minutes and then slowly transferred into the solution A while maintaining the temperature below −20° C. The mixture was stirred at below −20° C. for an additional hour before quenching the reaction by adding 20 mL of water. The reaction mixture was warmed to 0° C. and the pH was adjusted to about 7 by addition of 25% H₂SO₄ (11 mL). The mixture was further warmed to 20° C. and then diluted with 100 mL of ethyl acetate and 70 mL of water. The two phases that had formed were separated and the aqueous layer was extracted with 50 mL of ethyl acetate. The solvents THF and ethyl acetate were then replaced with ethanol, and the product 10b was precipitated out as a crystalline solid from ethanol with seeding at 35 to 40° C. After cooling to 0° C., the suspension was stirred for an additional hour and then the product 10b was filtered and washed with cold ethanol. The product was dried at 50-60° C. under vacuum to provide an off-white solid. Yield: 12.7 g, (90%). ¹H NMR (CDCl₃) δ 8.88 (d, J=2.4 Hz, 1H), 8.10 (dd, J=8.2, 2.4 Hz, 1H), 7.64 (1H), 7.61 (d, J=8.8 Hz, 1H), 7.55 (m, J=8.2, 6.2 Hz, 1H), 7.5 (d, J=8.0 Hz, 1H), 7.25 (dt, J=9.0, 2.3 Hz, 1H), 7.08 (d, J=8.0 Hz, 1H), 6.68 (dd, J=15.4, 9.4 Hz, 1H), 6.58 (d, J=9.6 Hz, 1H), 4.85 (dd, J=14.2, 7.2 Hz, 1H), 3.95 (dd, J=14.2, 7.1 Hz, 2H), 3.29 (m, 1H), 2.66 (m, J=12.0, 6.4 Hz, 1H), 2.33 (m, 2H), 1.76 (m, 4H), 1.30 (d, J=5.6 Hz, 3H), 1.19 (m, 4H), 1.14 (t, J=7.2 Hz, 3H), 0.98 (m, 1H), 0.84 (m, 1H). MS (EI) m/z: calcd. 492, actual 492.

Using an analogous procedure, 10a, 10c, 10d, 10e, and 10f were prepared by using the corresponding chloroformate in place of ethyl chloroformate in Step 5 of Scheme 1. The corresponding chloroformate include methylchloroformate for 10a, carbamoyl methyl chloroformate for 10c, chloroformate-acetic acid for 10d, chloroformate-acetic acid methyl ester for 10e and n-propyl chloroformate for 10f.

### Scheme 2

Scheme 2 outlines the conversion of either (R)- or (S)-propargylic alcohol 11 to the target compound.

The hydroxyl group of (R)-propargylic alcohol 11 was protected with tetra hydropyran (THP) followed by direct lithiation with n-butyl lithium (n-BuLi) and conversion to the ester. The O-THP protected ester was deprotected under acidic conditions to yield the ester with a free hydroxyl group 12, which was reacted with dienoic acid 13 to form compound 14 containing a triple bond, which was selectively reduced to form a double bond providing the intramolecular Diels-Alder precursor 15, which was thermally induced to initiate the Diels Alder reaction, which provided the diasteromeric mixture of the carboxylic acid 17, which was reduced to the aldehyde 18, which was further reacted with a diethylether 19 under the Emmons-Wadsworth reaction conditions to yield the ketal 20. The ketal 20 was deprotected under acidic conditions and subjected to reductive amination to yield the primary amine 21, which was treated with a chloroformate to yield the target compound 22, which was isolated as separate diastereomers.

Enantiomers of each of the separate diastereomers was synthesized by starting with the (S)-propargylic alcohol and following the same sequence of steps of described in Scheme 2 above.

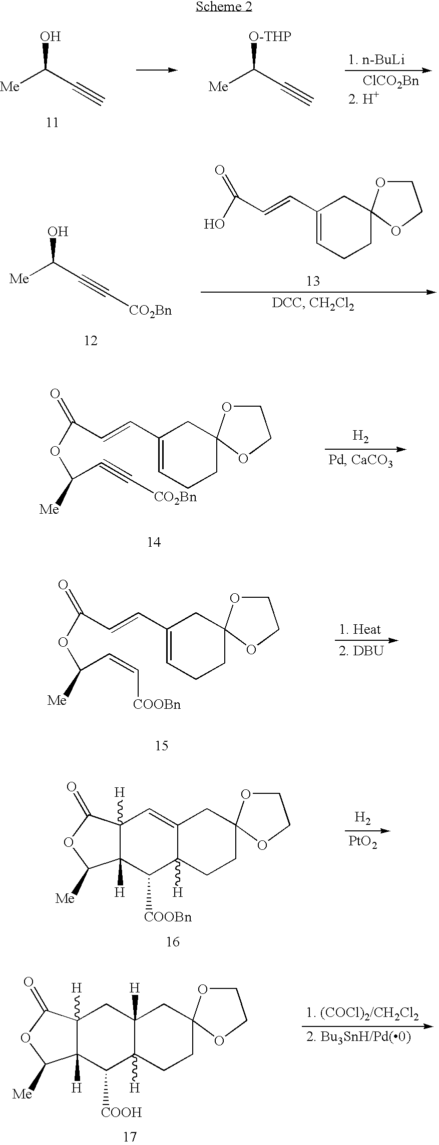

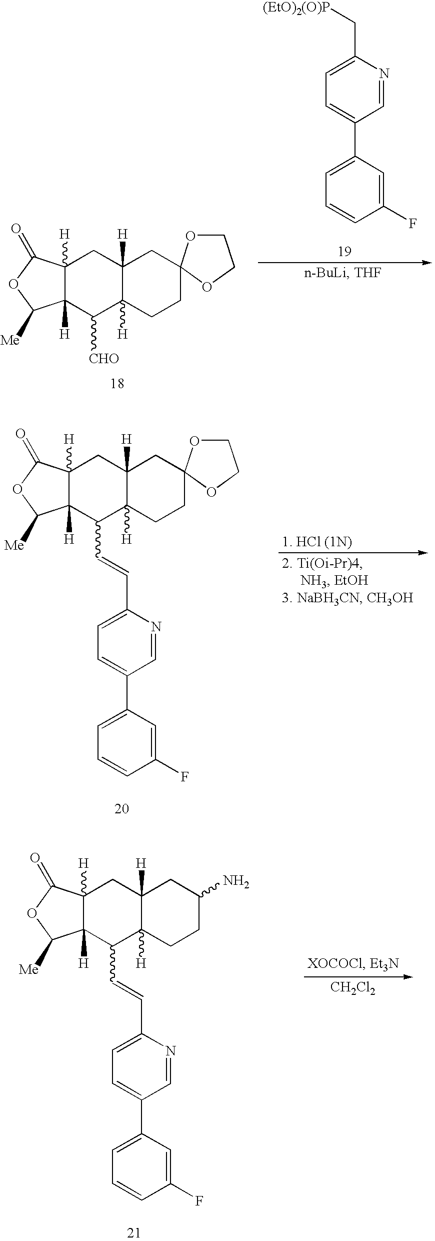

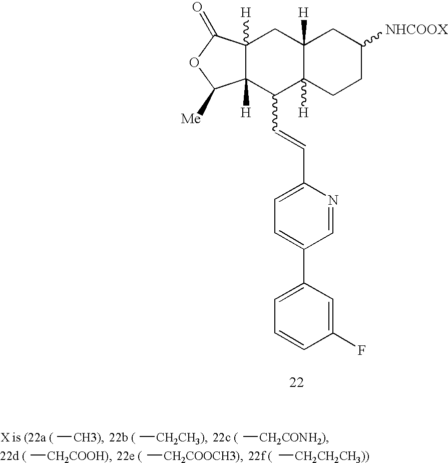

The compound numbers in the examples refer to the compound numbers in the schemes.
Preparation of: 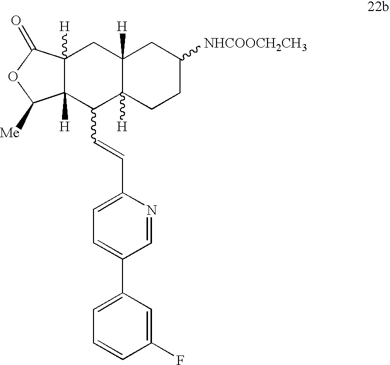
A.
Step 1: The hydroxyl group of R-propargylic alcohol 11 was protected with tetra hydropyran followed by direct lithiation with n-butyl lithium and conversion to the ester. The O-THP protected ester was deprotected under acidic conditions to yield the ester with a free hydroxyl group 12.
Step 2: The ester with a free hydroxyl group 12, was reacted with dienoic acid 13 to form compound 14, which has a triple bond.
Step 3: The triple bond of compound 14 was selectively reduced to form a double bond providing the intramolecular Diels-Alder precursor 15.
Step 4: The intramolecular Diels-Alder precursor 15, was thermally induced to initiate the Diels Alder reaction, which provided the diasteromeric mixture of the carboxylic acid 17.
Step 5: The diasteromeric mixture of the carboxylic acid 17, was reduced to the aldehyde 18.
Step 6: The aldehyde 18, was further reacted with a diethylether 19 under the Emmons-Wadsworth reaction conditions to yield the ketal 20.
Step 7: The ketal 20 was deprotected under acidic conditions and subjected to reductive amination to yield the primary amine 21.
Step 8: The primary amine 21, was treated with ethylchloroformate to yield the target compound 22b, which was isolated as separate diastereomers.
B.

Using an analogous procedure, 22a, 22c, 22d, 22e and 22f were prepared by using the corresponding chloroformate, which include methylchloroformate for 22a, carbamoyl methyl chloroformate for 22c, chloroformate-acetic acid for 22d, chloroformate-acetic acid methyl ester for 22e and n-propyl chloroformate for 22f.

C.

Enantiomers of each of the separate diastereomers was synthesized by starting with the (S)-propargylic alcohol and following the same sequence of steps of described in Scheme 2 above.

### Scheme 3

Scheme 3 outlines the conversion of a known monoketal derivative 23 (Johnson, J. et al. J. Am. Chem. Soc. 1962, 84, 2181, 2191) to a tricyclic ketone, which is converted to the final products using Emmons-Wadsworth reaction followed by other identical reaction steps as shown in Scheme 2.

The known monoketal derivative 23 (Johnson, J. et. al. J. Am. Chem. Soc. 1962, 84, 2181, 2191) can be converted to the enone 24 using a standard dehydrogenation protocol. Cyanide conjugate addition to the enone 24 followed by a silyl enol ether mediated aldol reaction, provides the intermediate 27 which can be converted to the tricyclic ketone 28 by acid mediated hydrolysis. Wittig reaction of ketone 28 followed by hydrolysis of the resultant enol ether furnished the aldehyde 29, which reacts under Emmons-Wadsworth reaction conditions to form compound 30, which can be converted to the final product 32 using the protocol as described in Scheme 2. 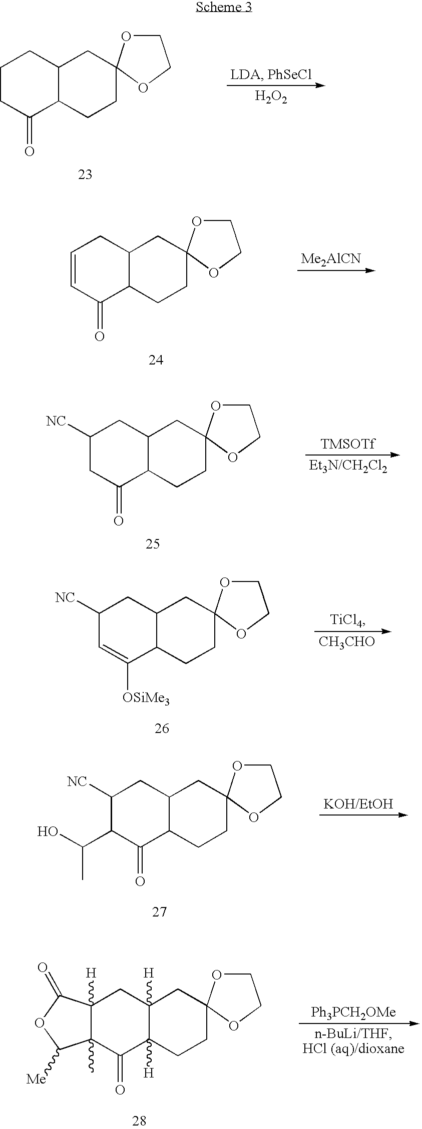 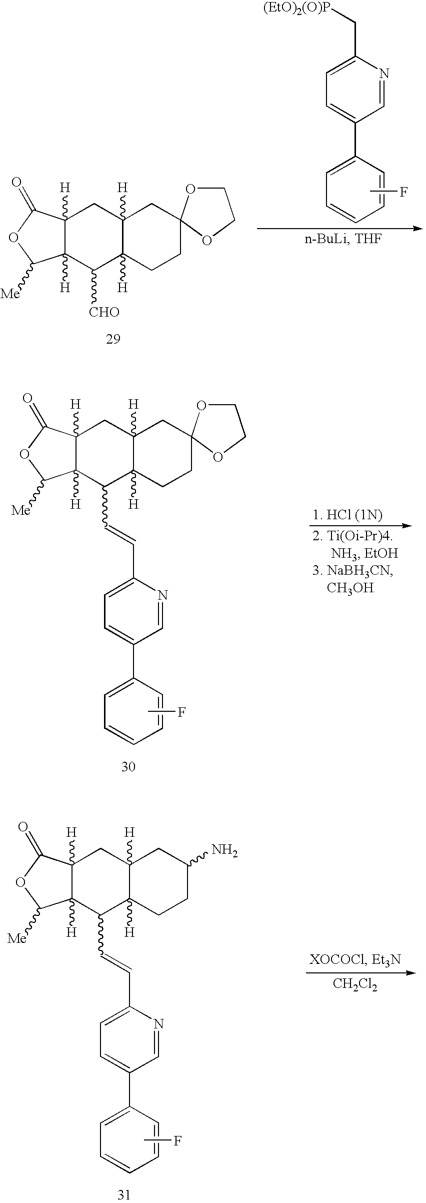 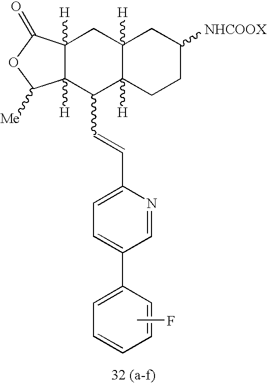
Step 1: The known monoketal derivative 23 (Johnson, J. et. al J. Am. Chem. Soc. 1962, 84, 2181, 2191) is reacted with a strong base lithium diisopropylamide (LDA) and phenylselenyl chloride (PhSeCl) and hydrogen peroxide to form the enone 24.
Step 2: Enone 24 is treated with the organic aluminum cyanide, dimethylaluminum cyanide, to form 25, which is then reacted with a silyation reagent, trimethylsilyl triflate and the catalyst TiCl₄ for the aldol coupling of 25 with Acetaldehyde, which can provide the intermediate 27 which upon hydrolysis using potassium hydroxide in ethanol forms the tricyclic ketone 28.
Step 3: The tricyclic ketone 28 can be subjected to the Wittig reaction by reacting 28 with Ph₃PCH₂Ome, nBuLi in tetrahydrofuran, to form an enol ether, which can be hydrolysed using hydrochloric acid in dioxane to yield the aldehyde 29.
Step 4: The aldehyde 29 can be reacted under Emmons-Wadsworth reaction conditions to form compound 30.
Step 5: Compound 30 can be converted to the final products 32 a-f, which are the same as products or stereoisomers of 32 a-f from Scheme 2, using the same protocol following the Emmons-Wadsworth reaction as described in Scheme 2.

Further embodiments of the invention encompass the administration of compounds of the invention along with at least one additional cardiovascular agent. The contemplated additional cardiovascular agent is one that differs in either atomic make up or arrangement from the compounds of the invention. Additional cardiovascular agents that can be used in combination with the novel compounds of this invention include drugs, which have anti-thrombotic, anti-platelet aggregation, antiatherosclerotic, antirestenotic and/or anti-coagulant activity. Such drugs are useful in treating thrombosis-related diseases including thrombosis, atherosclerosis, restenosis, hypertension, angina pectoris, angiogenesis related disorders, arrhythmia, a cardiovascular or circulatory disease or condition, heart failure, myocardial infarction, glomerulonephritis, thrombotic stroke, thromboembolytic stroke, peripheral vascular diseases, cerebral ischemia, rheumatoid arthritis, rheumatism, astrogliosis, a fibrotic disorder of the liver, kidney, lung or intestinal tract, systemic lupus erythematosus, multiple sclerosis, osteoporosis, glomerulonephritis, renal disease, acute renal failure, chronic renal failure, renal vascular homeostasis, renal ischemia, bladder inflammation, diabetes, diabetic neuropathy, cerebral stroke, cerebral ischemia, nephritis, cancer, melanoma, renal cell carcinoma, neuropathy and/or malignant tumors, neurodegenerative and/or neurotoxic diseases, conditions, or injuries, inflammation, asthma, glaucoma, macular degeneration, psoriasis, endothelial dysfunction disorders of the liver, kidney or lung inflammatory disorders of the lungs and gastrointestinal tract, respiratory tract disease or condition, radiation fibrosis, endothelial dysfunction, periodontal diseases or wounds or a spinal cord injury, or a symptom or result thereof, as well as other disorders in which thrombin and its receptor play a pathological role. Suitable cardiovascular agents are selected from the group consisting of thromboxane A2 biosynthesis inhibitors such as aspirin; thromboxane antagonists such as seratrodast, picotamide and ramatroban; adenosine diphosphate (ADP) inhibitors such as clopidogrel; cyclooxygenase inhibitors such as aspirin, meloxicam, rofecoxib and celecoxib; angiotensin antagonists such as valsartan, telmisartan, candesartran, irbesartran, losartan and eprosartan; endothelin antagonists such as tezosentan; phosphodiesterase inhibitors such as milrinoone and enoximone; angiotensin converting enzyme (ACE) inhibitors such as captopril, enalaprif, enaliprilat, spirapril, quinapril, perindopril, ramipril, fosinopril, trandolapril, lisinopril, moexipril and benazapril; neutral endopeptidase inhibitors such as candoxatril and ecadotril; anticoagulants such as ximelagatran, fondaparin and enoxaparin; diuretics such as chlorothiazide, hydrochlorothiazide, ethacrynic acid, furosemide and amiloride; platelet aggregation inhibitors such as abciximab and eptifibatide; and GP IIb/IIIa antagonists.

Preferred types of drugs for use in combination with the novel compounds of this invention are thromboxane A2 biosynthesis inhibitors, GP IIb/IIIa antagonists, thromboxane antagonists, adenosine diphosphate inhibitors, cyclooxygenase inhibitors, angiotensin antagonists, endothelin antagonists, angiotensin converting enzyme inhibitors, neutral endopeptidase inhibitors, anticoagulants, diuretics, and platelet aggregation inhibitors. Especially preferred for use in the combinations are aspirin, cangrelor and/or clopidogrel bisulfate.

When the invention comprises a combination of compounds of the invention and another cardiovascular agent, the two active components may be co-administered simultaneously or sequentially, or a single pharmaceutical composition comprising compounds of the invention and another cardiovascular agent in a pharmaceutically acceptable carrier can be administered. The components of the combination can be administered individually or together in any conventional dosage form such as capsule, tablet, powder, cachet, suspension, solution, suppository, nasal spray, etc. The dosage of the cardiovascular agent can be determined from published material, and may range from 1 to 1000 mg per dose.

In this specification, the term “at least one compound of the invention” means that one to three different compounds of this invention may be used in a pharmaceutical composition or method of treatment. Preferably one compound of the invention is used. Similarly, the term “one or more additional cardiovascular agents” means that one to three additional drugs may be administered in combination with a compound of the invention; preferably, one additional compound is administered in combination with a compound of the invention. The additional cardiovascular agents can be administered sequentially or simultaneously with reference to the compounds of the invention.

When separate compounds of the invention and the other cardiovascular agents are to be administered as separate compositions, they can be provided in a kit comprising in a single package, one container comprising a compounds of the invention in a pharmaceutically acceptable carrier, and a separate container comprising another cardiovascular agent in a pharmaceutically acceptable carrier, with the compounds of the invention and the other cardiovascular agent being present in amounts such that the combination is therapeutically effective. A kit is advantageous for administering a combination when, for example, the components must be administered at different time intervals or when they are in different dosage forms.

The activity of the compounds of the invention can be determined by the following procedures.

In Vitro Testing Procedure for Thrombin Receptor Antagonists:

Preparation of [³H]haTRAP

A(pF-F)R(ChA)(hR)(I₂—Y)—NH₂ (1.03 mg) and 10% Pd/C (5.07 mg) were suspended in DMF (250 μl) and diisopropylethylamine (10 μl). The vessel was attached to the tritium line, frozen in liquid nitrogen and evacuated. Tritium gas (342 mCi) was then added to the flask, which was stirred at room temperature for 2 hours. At the completion of the reaction, the excess tritium was removed and the reacted peptide solution was diluted with DMF (0.5 ml) and filtered to remove the catalyst. The collected DMF solution of the crude peptide was diluted with water and freeze dried to remove the labile tritium. The solid peptide was redissolved in water and the freeze drying process repeated. The tritiated peptide ([³H]haTRAP) was dissolved in 0.5 ml of 0.1% aqueous TFA and purified by HPLC using the following conditions: column, Vydac™ C18, 25 cm×9.4 mm I.D.; mobile phase, (A) 0.1% TFA in water, (B) 0.1% TFA in CH₃CN; gradient, (A/B) from 100/0 to 40/60 over 30 min; flow rate, 5 ml/min, detection, UV at 215 nm. The radiochemical purity of [³H]haTRAP was 99% as analyzed by HPLC. A batch of 14.9 mCi at a specific activity of 18.4 Ci/mmol was obtained.

Preparation of Platelet Membranes

Platelet membranes were prepared using a modification of the method of Natarajan et al. (Natarajan et al, Int. J. Peptide Protein Res. 45:145-151 (1995)) from 20 units of platelet concentrates obtained from the North Jersey Blood Center (East Orange, N.J.) within 48 hours of collection. All steps were carried out at 4° C. under approved biohazard safety conditions. Platelets were centrifuged at 100×g for 20 minutes at 4° C. to remove red cells. The supernatants were decanted and centrifuged at 3000×g for 15 minutes to pellet platelets. Platelets were re-suspended in 10 mM Tris-HCl, pH 7.5, 150 mM NaCl, 5 mM EDTA, to a total volume of 200 ml and centrifuged at 4400×g for 10 minutes. This step was repeated two additional times. Platelets were re-suspended in 5 mM Tris-HCl, pH 7.5, 5 mM EDTA to a final volume of approximately 30 ml and were homogenized with 20 strokes in a Dounce™ homogenizer. Membranes were pelleted at 41,000×g, re-suspended in 40-50 ml 20 mM Tris-HCl, pH 7.5, 1 mM EDTA, 0.1 mM dithiothreitol, and 10 ml aliquots were frozen in liquid N2 and stored at −80° C. To complete membrane preparation, aliquots were thawed, pooled, and homogenized with 5 strokes of a Dounce homogenizer. Membranes were pelleted and washed 3 times in 10 mM triethanolamine-HCl, pH 7.4, 5 mM EDTA, and re-suspended in 20-25 ml 50 mM Tris-HCl, pH 7.5, 10 mM MgCl₂, 1 mM EGTA, and 1% DMSO. Aliquots of membranes were frozen in liquid N2 and stored at −80° C. Membranes were stable for at least 3 months. 20 units of platelet concentrates typically yielded 250 mg of membrane protein. Protein concentration was determined by a Lowry assay (Lowry et al., J. Biol. Chem., 193:265-275 (1951)).

High Throughput Thrombin Receptor Radioligand Binding Assay

Thrombin receptor antagonists were screened using a modification of the thrombin receptor radioligand binding assay of Ahn et al. (Ahn et al., Mol. Pharmacol., 51:350-356 (1997)). The assay was performed in 96 well Nunc plates (Cat. No. 269620) at a final assay volume of 200 μl. Platelet membranes and [³H]haTRAP were diluted to 0.4 mg/ml and 22.2 nM, respectively, in binding buffer (50 mM Tris-HCl, pH 7.5, 10 mM MgCl₂, 1 mM EGTA, 0.1% BSA). Stock solutions (10 mM in 100% DMSO) of test compounds were further diluted in 100% DMSO. Unless otherwise indicated, 10 μl of diluted compound solutions and 90 μl of radioligand (a final concentration of 10 nM in 5% DMSO) were added to each well, and the reaction was started by the addition of 100 μl of membranes (40 μg protein/well). The binding was not significantly inhibited by 5% DMSO. Compounds were tested at three concentrations (0.1, 1 and 10 μM). The plates were covered and vortex-mixed gently on a Lab-Line™ Titer Plate Shaker for 1 hour at room temperature. Packard UniFilter™ GF/C filter plates were soaked for at least 1 hour in 0.1% polyethyleneimine. The incubated membranes were harvested using a Packard FilterMate™ Universal Harvester and were rapidly washed four times with 300 μl ice cold 50 mM Tris-HCl, pH 7.5, 10 mM MgCl₂, 1 mM EGTA. MicroScint™ 20 scintillation cocktail (25 μl) was added to each well, and the plates were counted in a Packard TopCount™ Microplate Scintillation Counter. The specific binding was defined as the total binding minus the nonspecific binding observed in the presence of excess (50 μM) unlabeled haTRAP. The % inhibition by a compound of [³H]haTRAP binding to thrombin receptors was calculated from the following relationship:

$$\% Inhibition = \frac{Total binding \text{-} Binding in the presence of a test compound}{Total binding \text{-} Nonspecific binding} \times 100$$

Materials

A(pF-F)R(ChA)(hR)Y—NH₂ and A(pF-F)R(ChA)(hR)(I₂—Y)—NH₂, were custom synthesized by AnaSpec Inc. (San Jose, Calif.). The purity of these peptides was >95%. Tritium gas (97%) was purchased from EG&G Mound, Miamisburg, Ohio. The gas was subsequently loaded and stored on an IN/US Systems Inc. Trisorber. MicroScint™ 20 scintillation cocktail was obtained from Packard Instrument Co.

Cannabinoid CB₂ Receptor Binding Assay

Binding to the human cannabinoid CB₂ receptor was carried out using the procedure of Showalter, et al., (1996, J. Pharmacol Exp Ther. 278(3), 989-99), with minor modifications. All assays were carried out in a final volume of 100 ul. Test compounds were re-suspended to 10 mM in DMSO, then serially diluted in 50 mM Tris, pH 7.1, 3 mM MgCl₂, 1 mM EDTA, 50% DMSO. Aliquots (10 ul) of each diluted sample were then transferred into individual wells of a 96-well microtiter plate. Membranes from human CB₂ transfected CHO/Ki cells (Receptor Biology, Inc) were re-suspended in binding buffer (50 mM Tris, pH 7.1, 3 mM MgCl₂, 1 mM EDTA, 0.1% fatty acid free bovine serum albumin), then added to the binding reaction (˜15 ug in 50 ul per assay). The reactions were initiated with the addition of [³H] CP-55, 940 diluted in binding buffer (specific activity=180 Ci/mmol; New England Nuclear, Boston, Mass.). The final ligand concentration in the binding reaction was 0.48 nM. Following incubation at room temperature for 2 hours, membranes were harvested by filtration through pretreated (0.5% polyethylenimine; Sigma P-3143) GF-C filter plates (Unifilter-96, Packard) using a TomTec™ Mach 3U 96-well cell harvester (Hamden, Conn.). Plates were washed 10 times in 100 ul binding buffer, and the membranes allowed to air dry. Radioactivity on membranes was quantitated following addition of Packard Omniscintim 20 scintillation fluid using a TopCount™ NXT Microplate Scintillation and Luminescence Counter (Packard, Meriden, Conn.). Non-linear regression analysis was performed using Prism™ 20b, (GraphPad Software, San Diego, Calif.).

What is claimed is:

1. A compound represented by any of the following structural formulas: 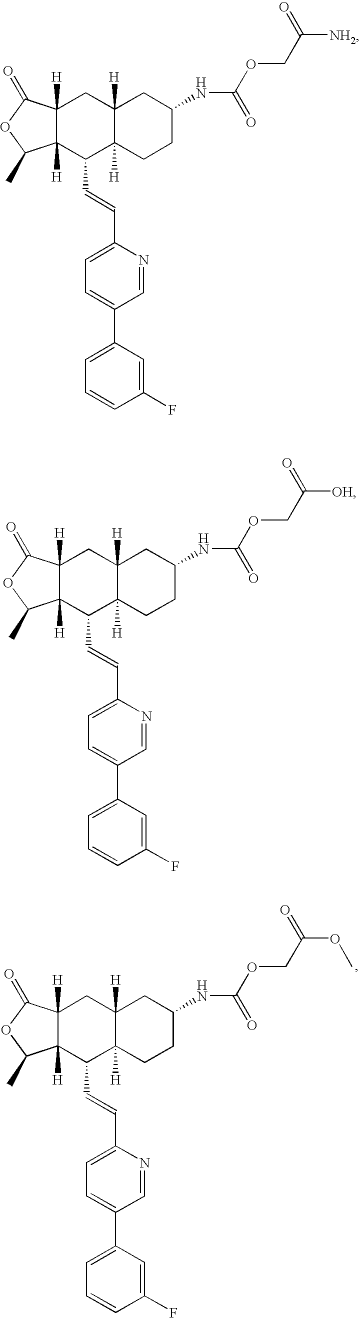 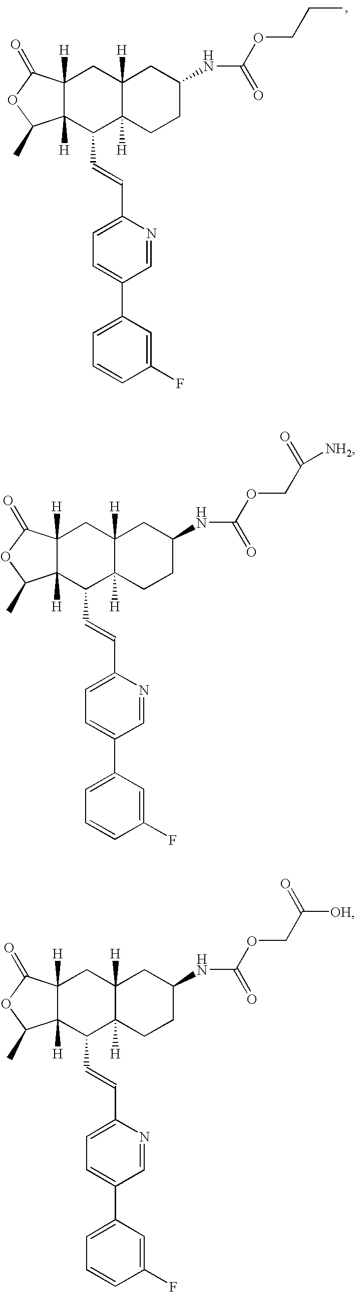 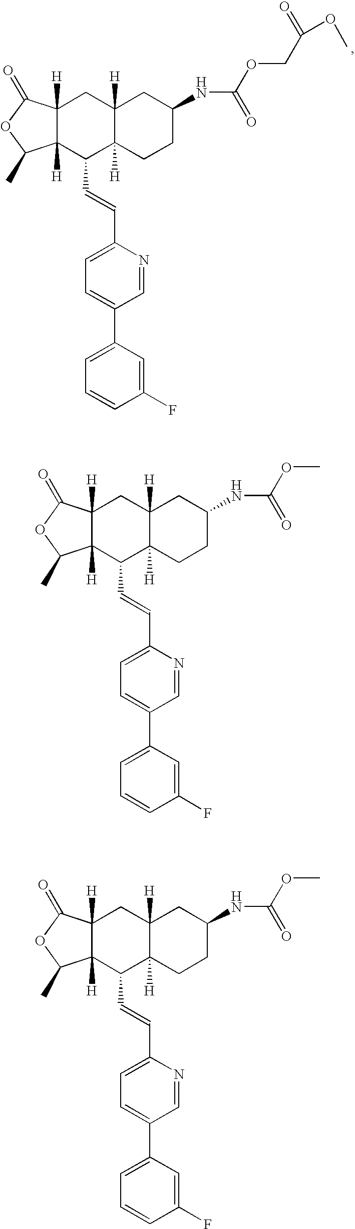 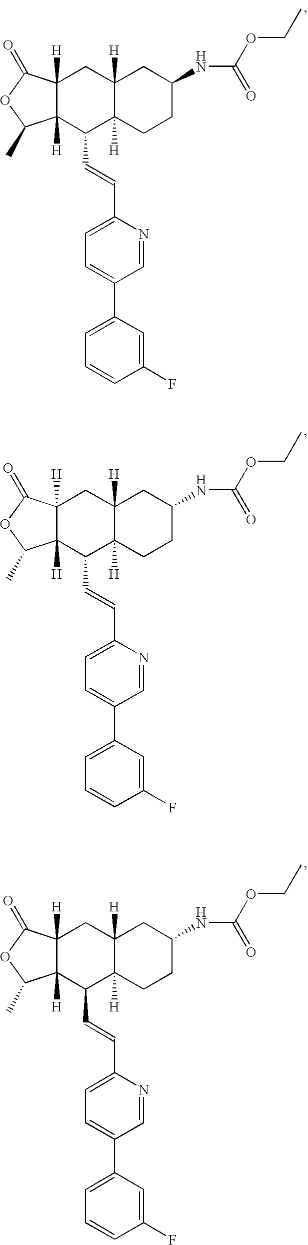 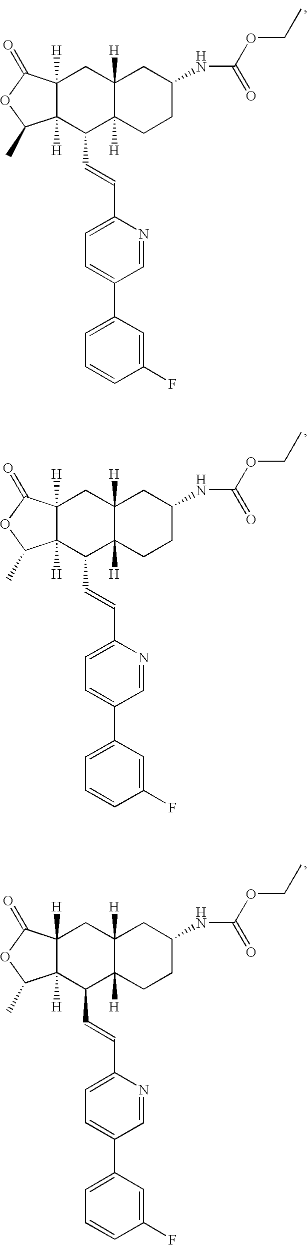 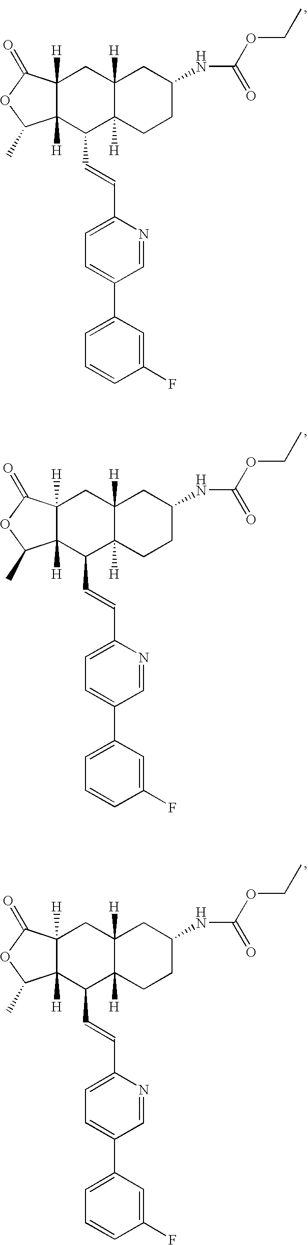 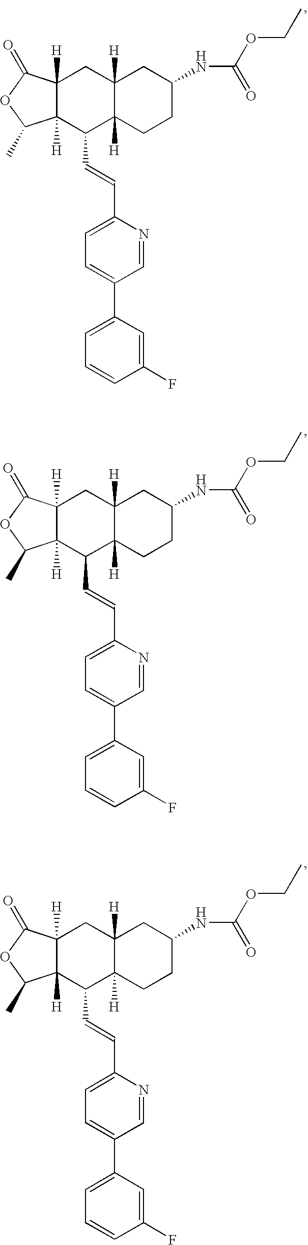 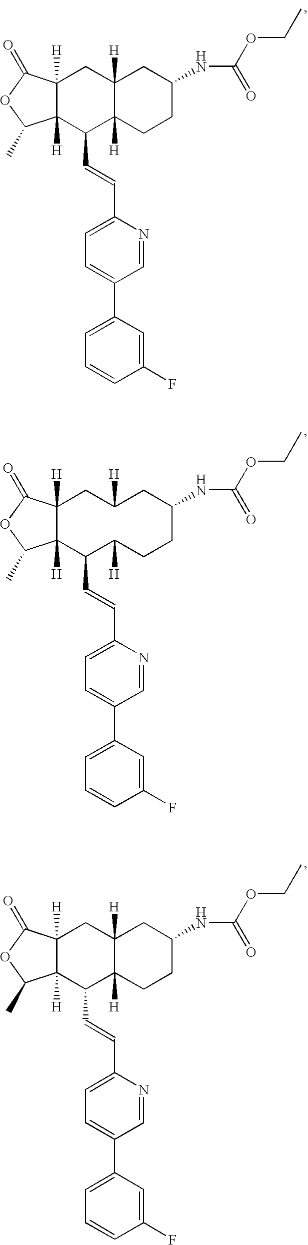 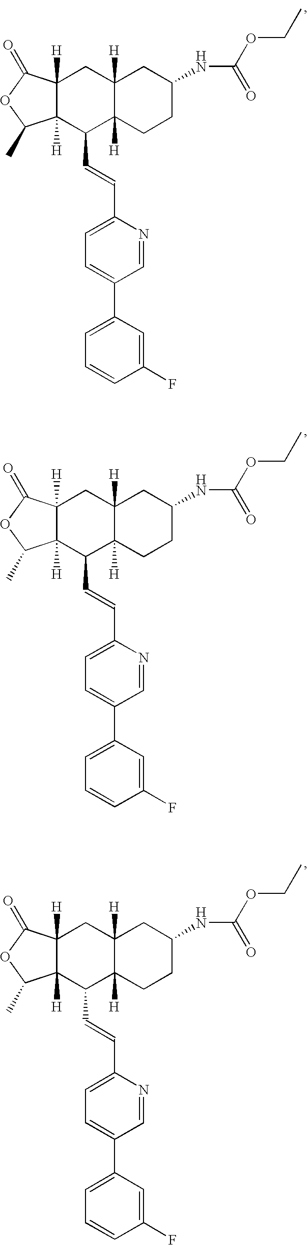 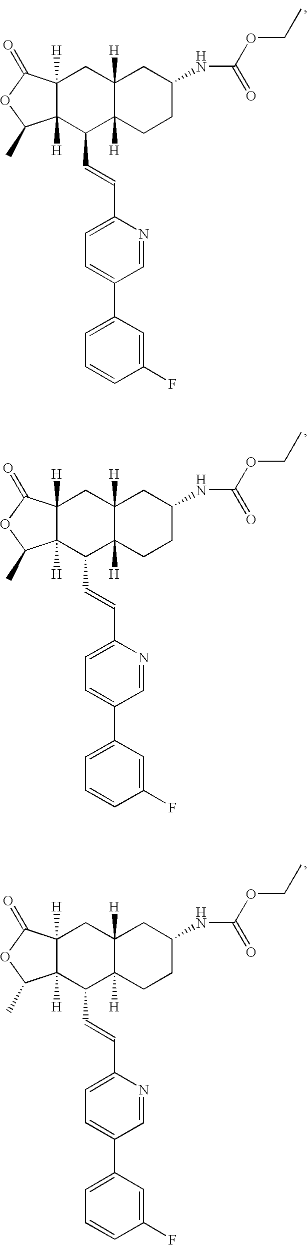 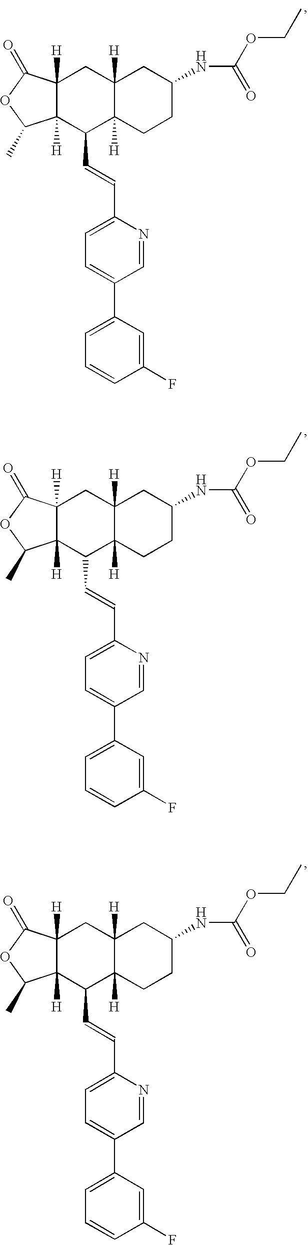 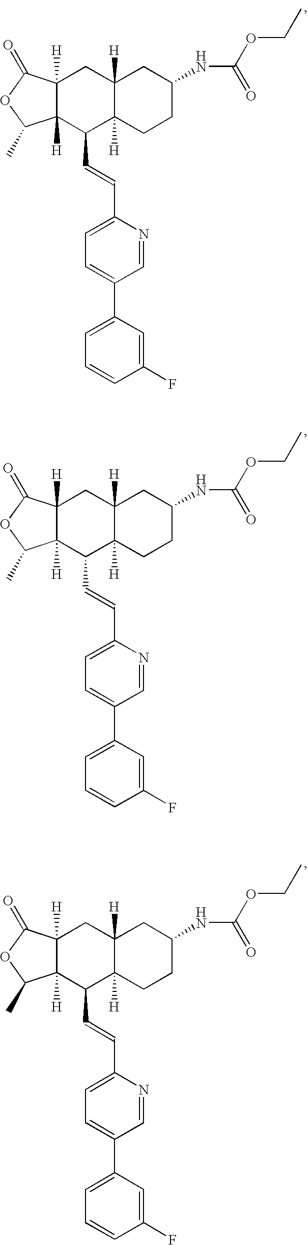 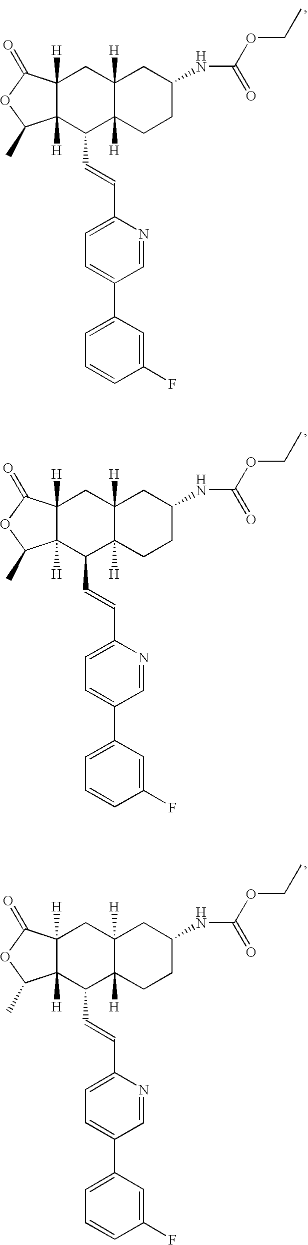 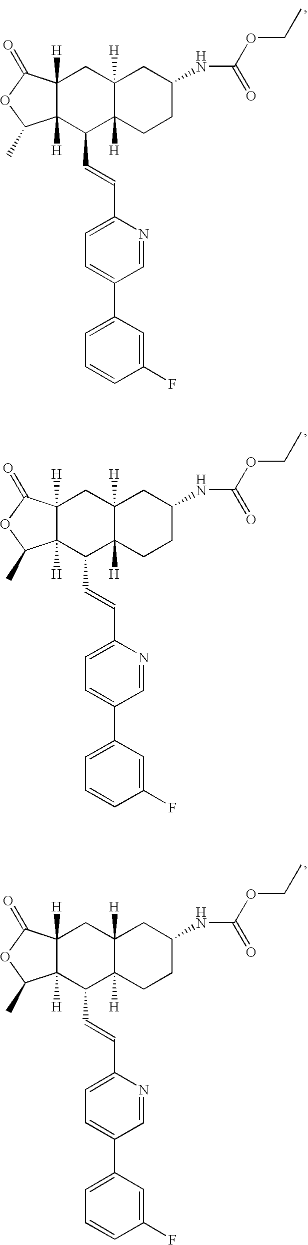 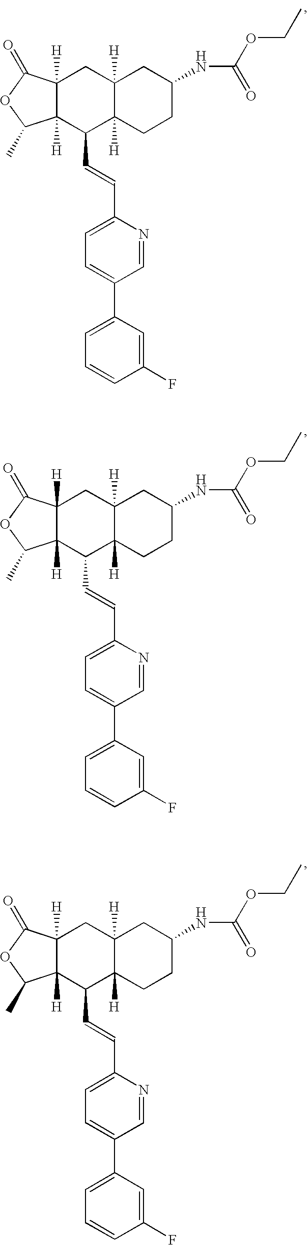 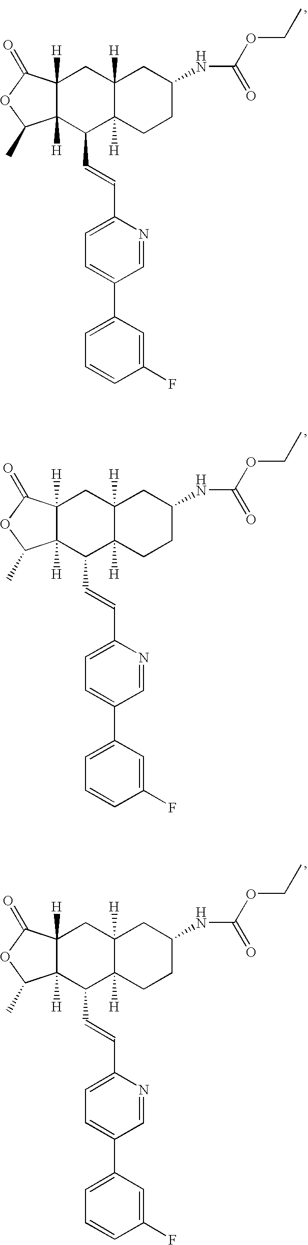 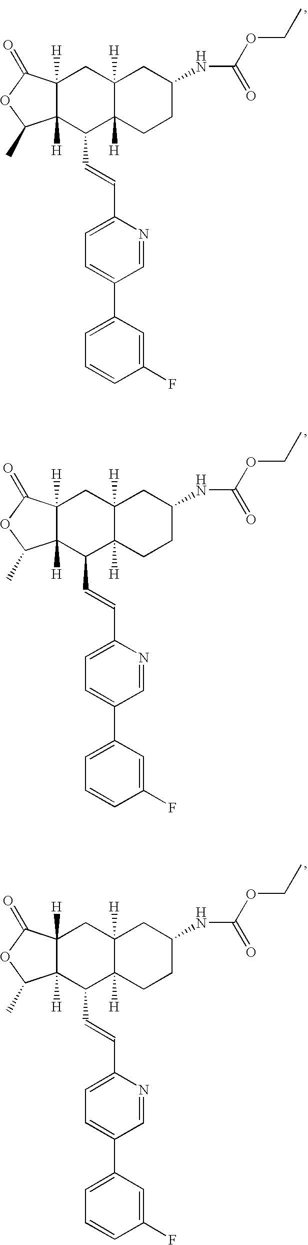 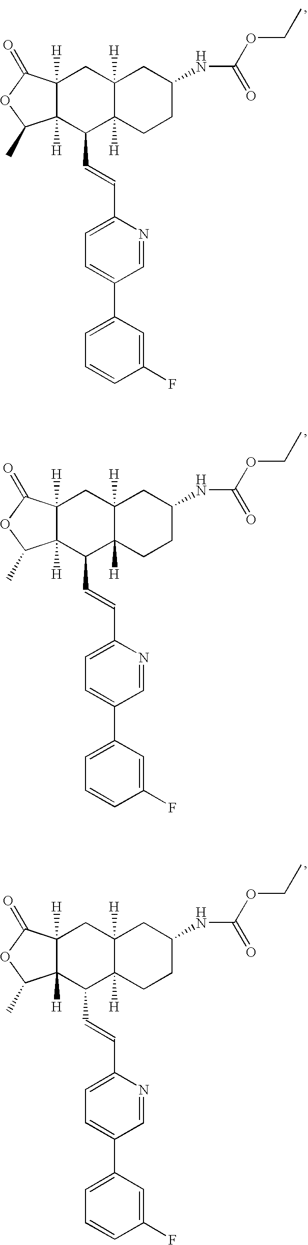 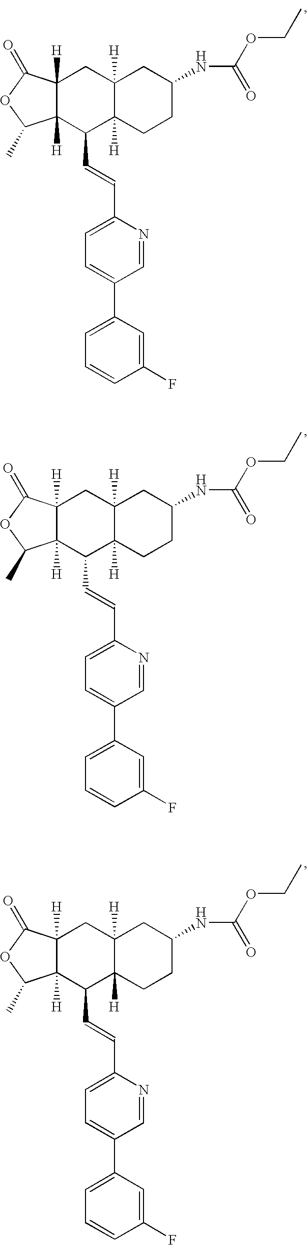 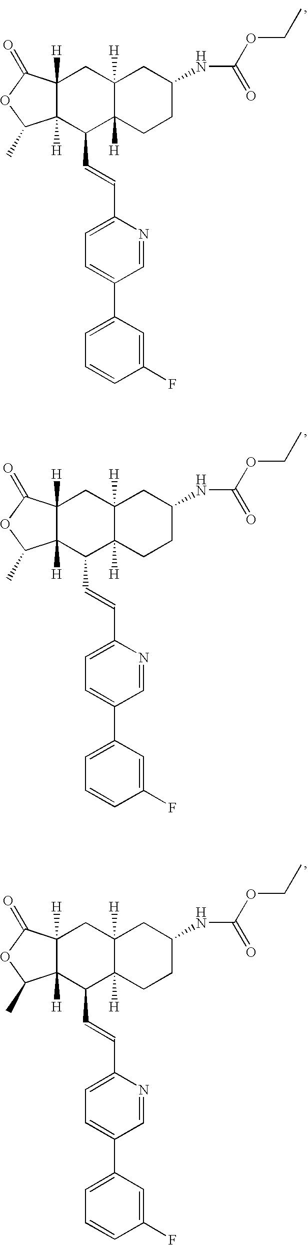 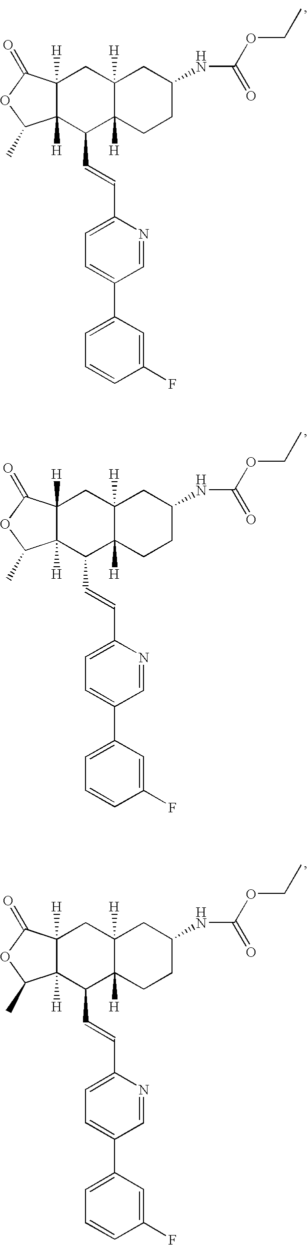 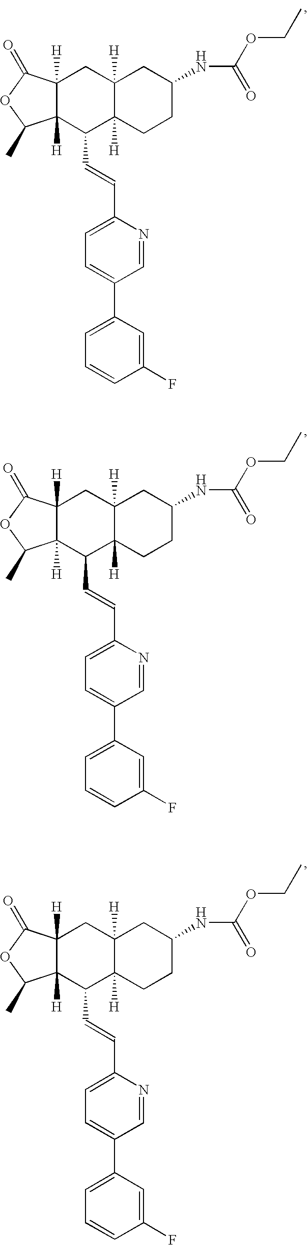 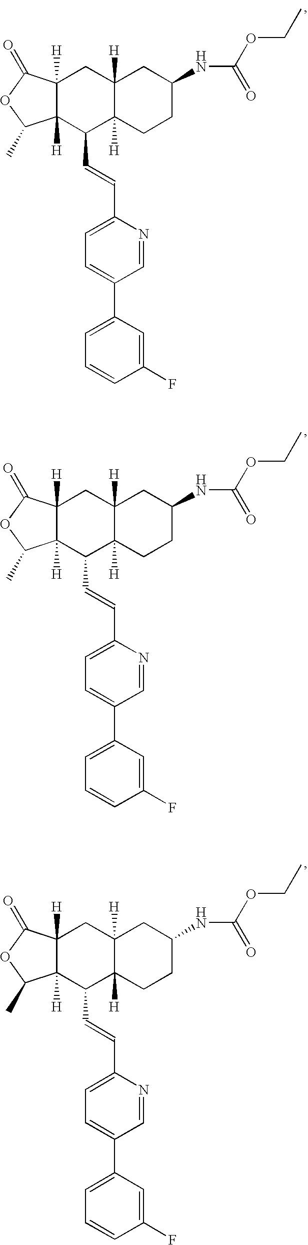 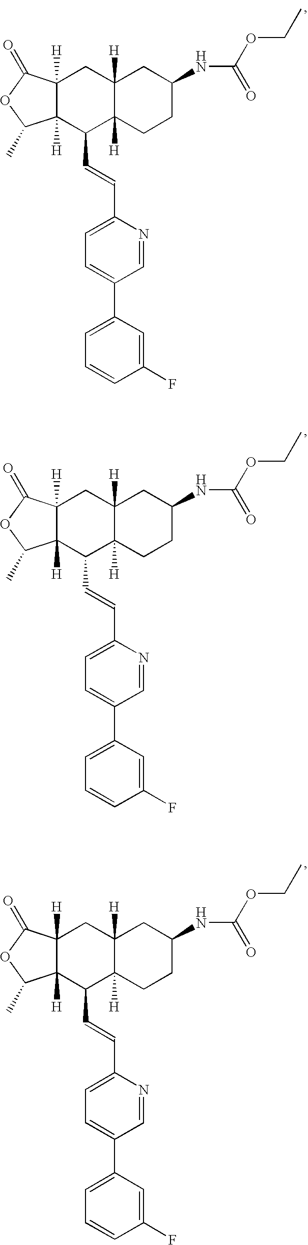 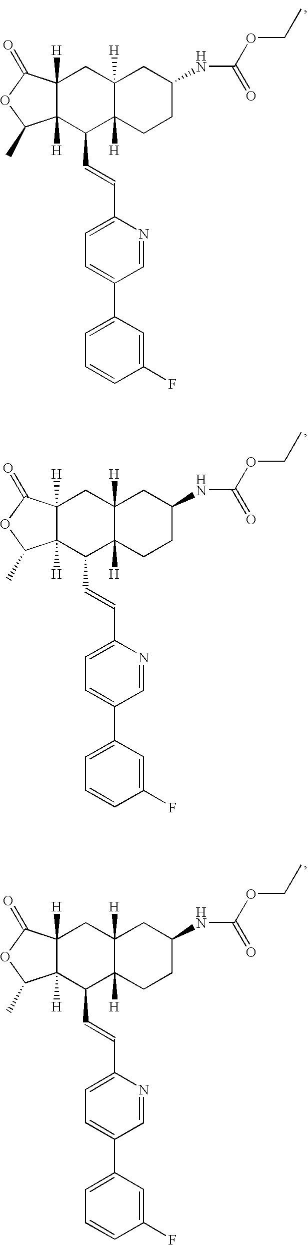 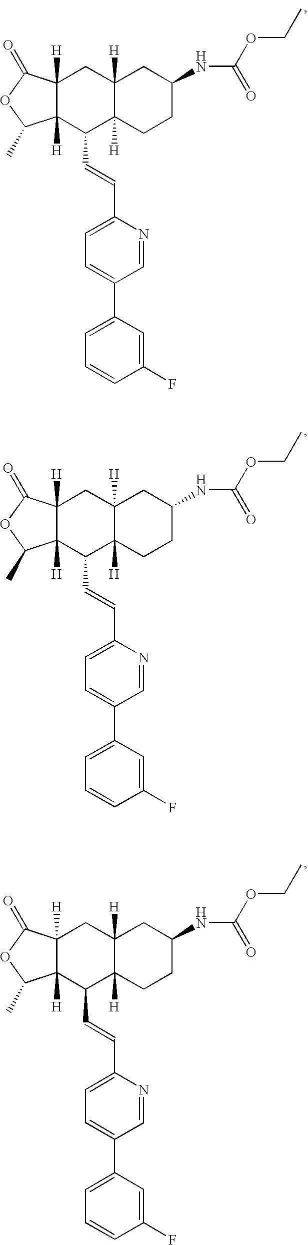 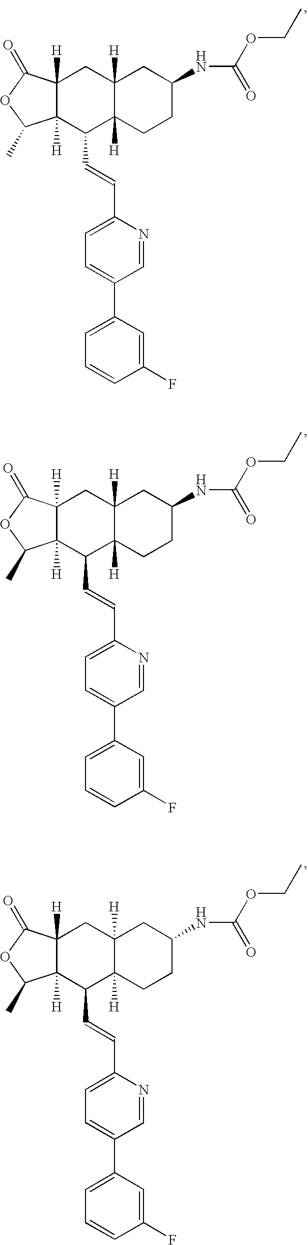 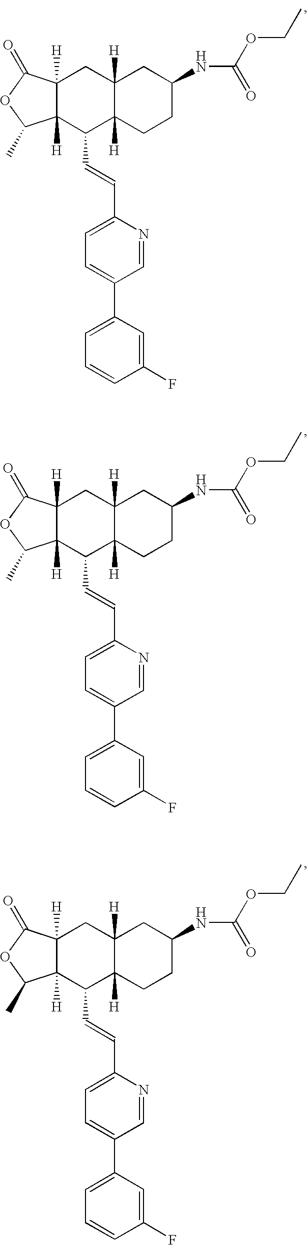 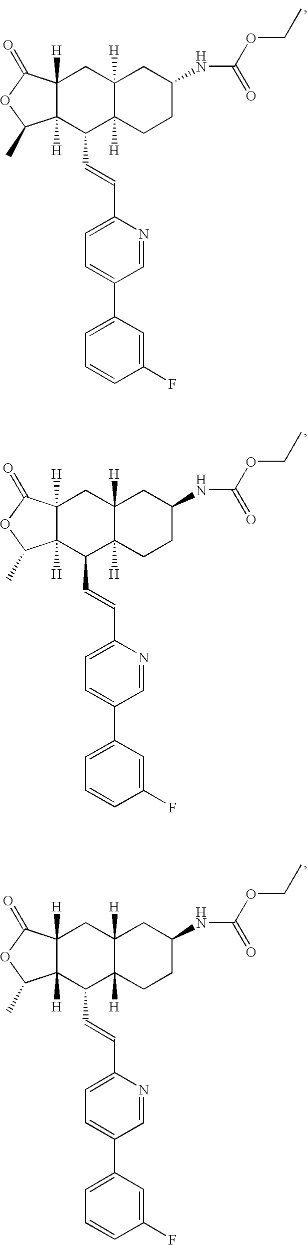 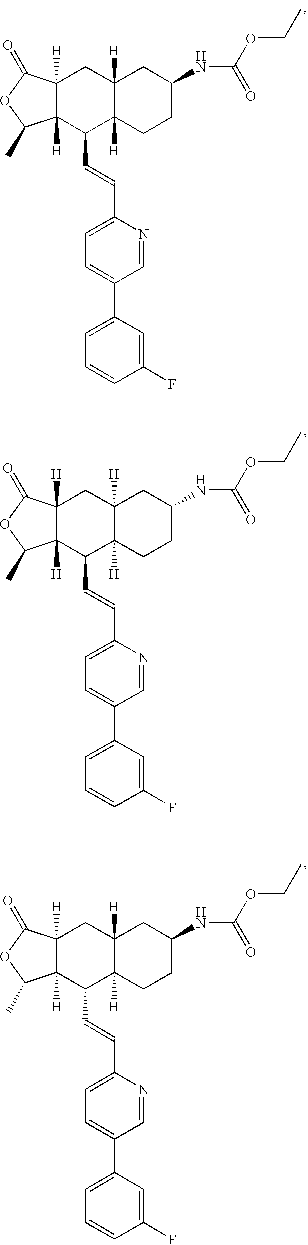 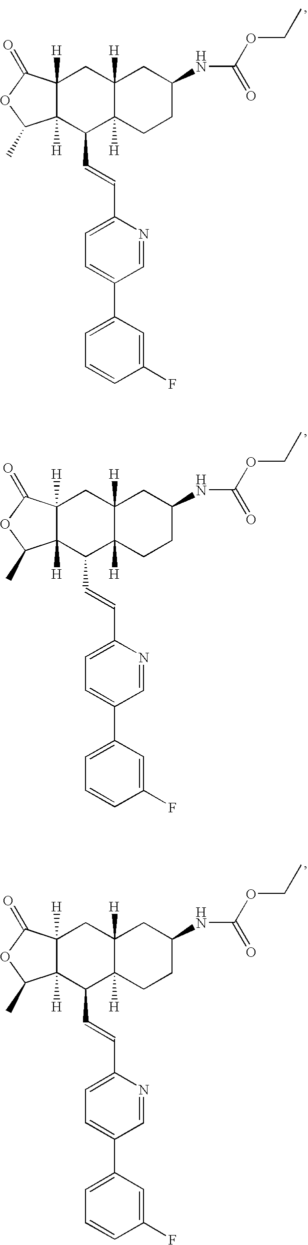 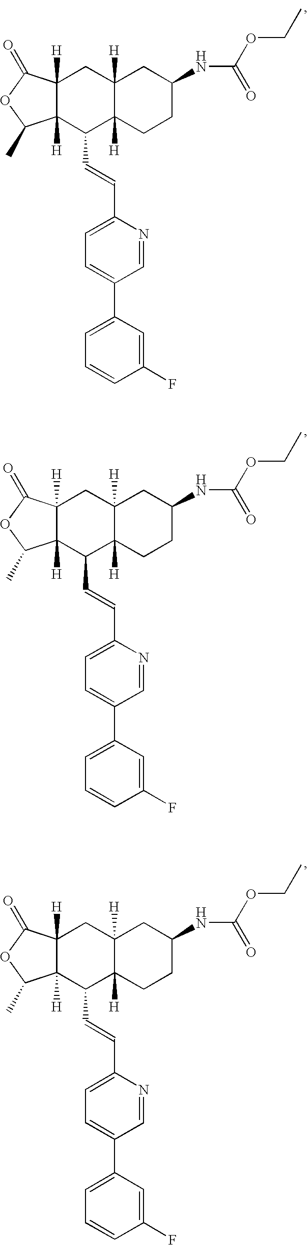 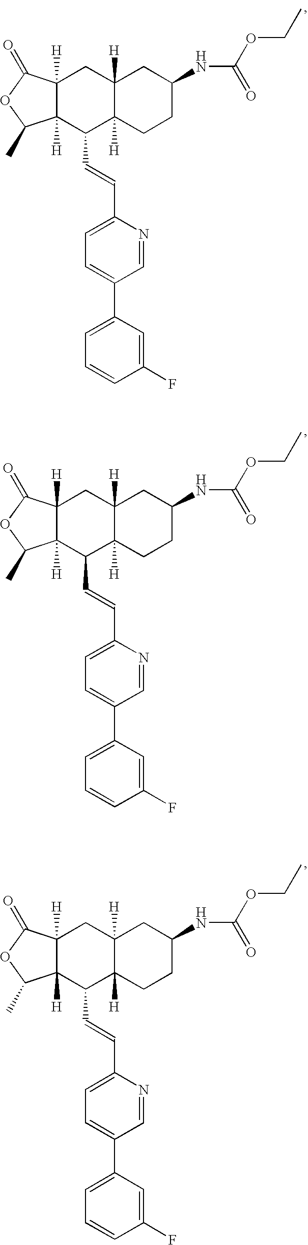 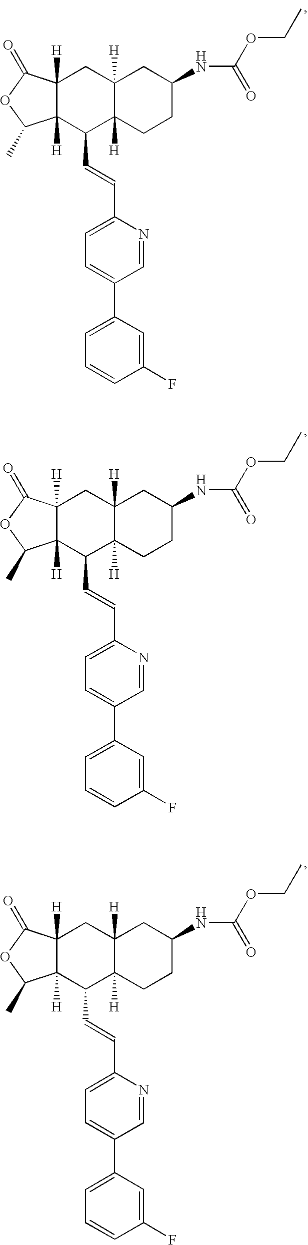 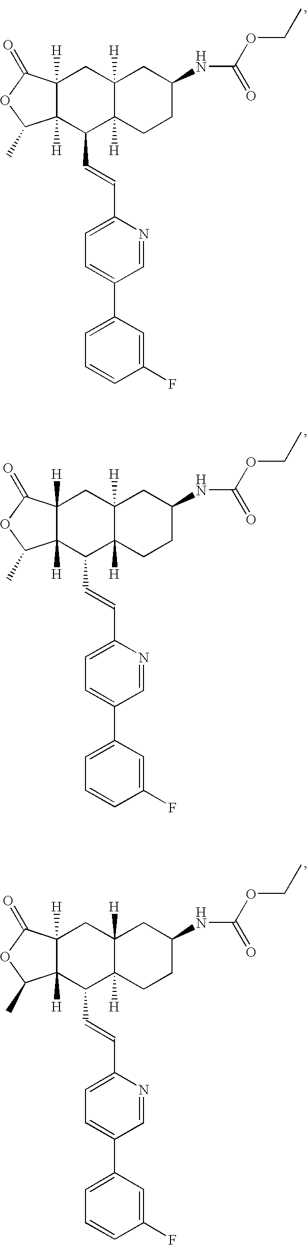 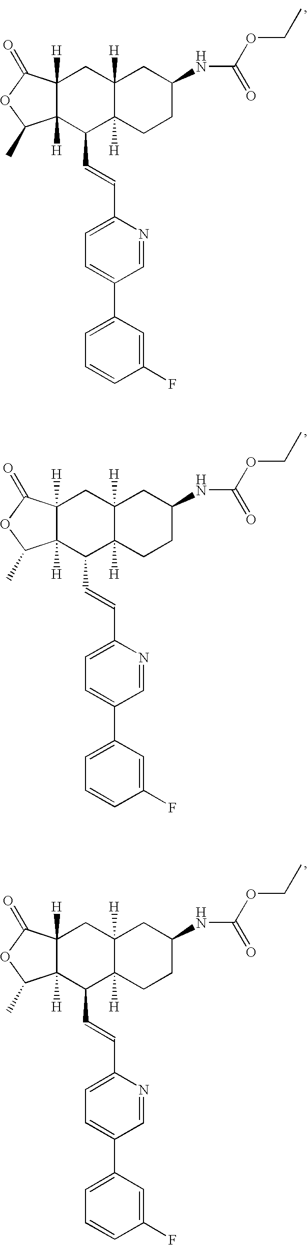 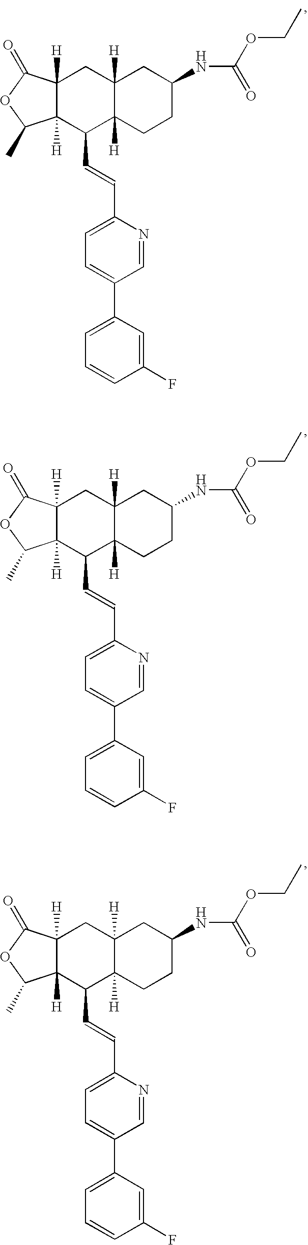 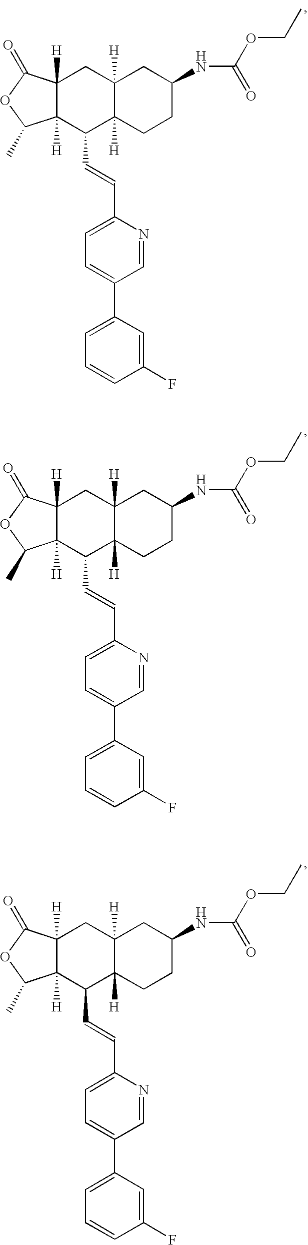 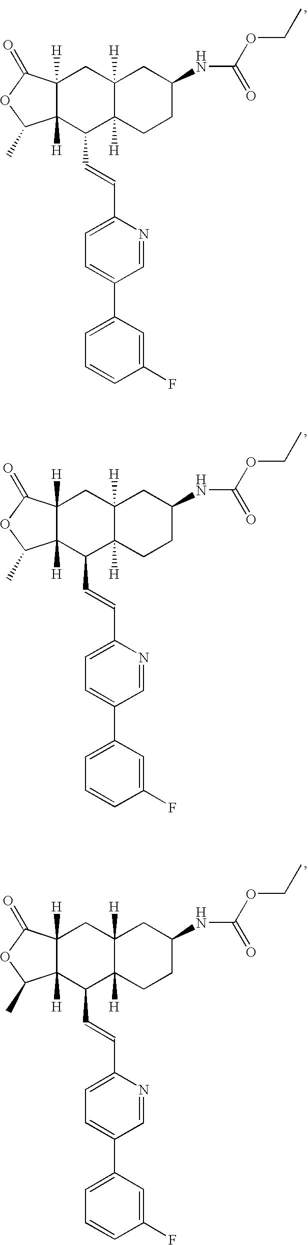 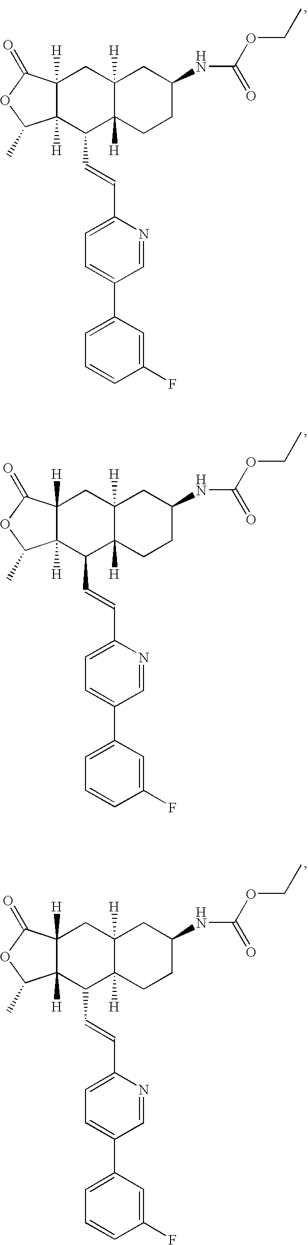 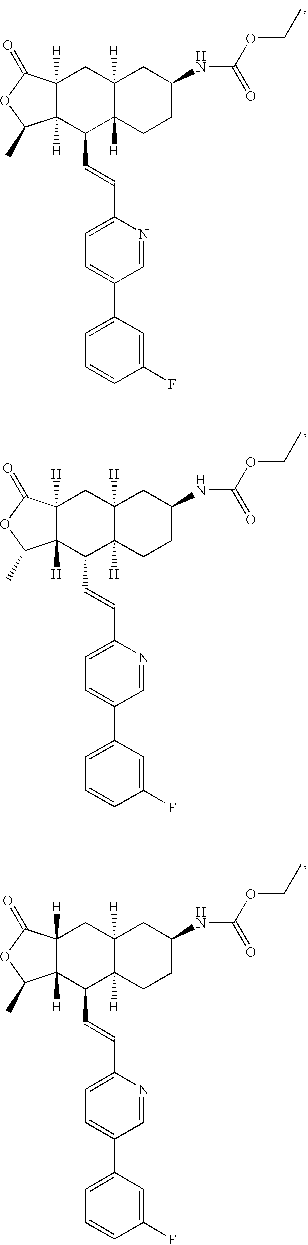 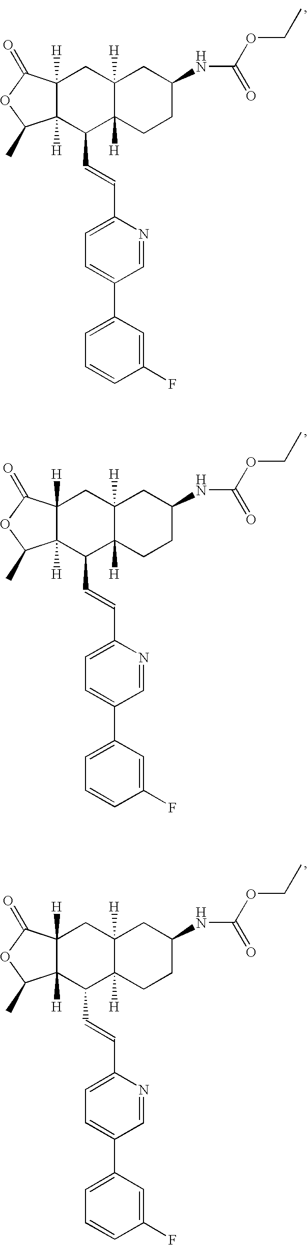 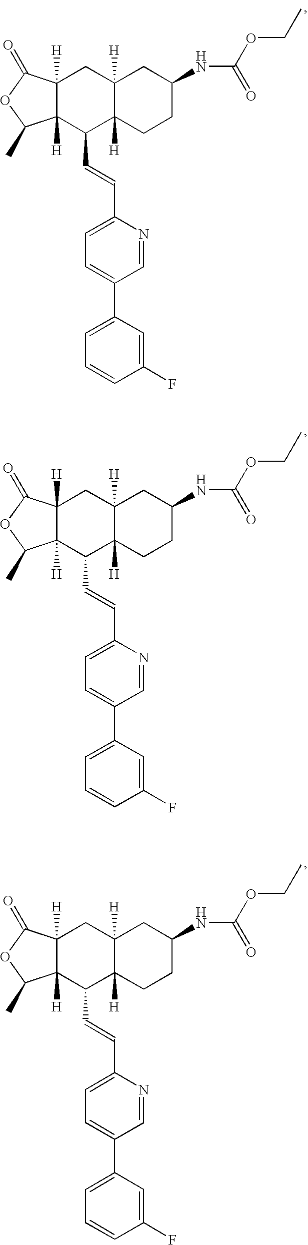 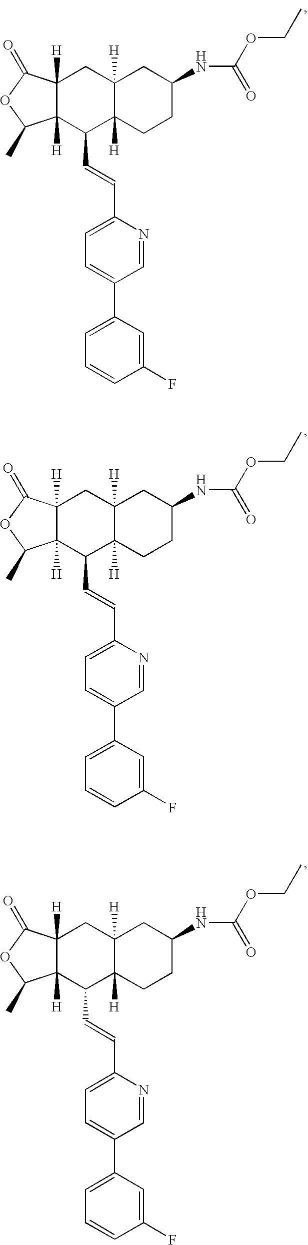 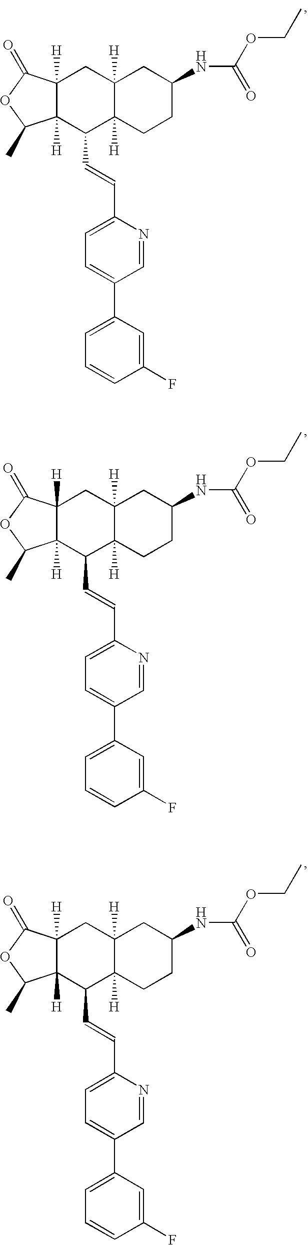 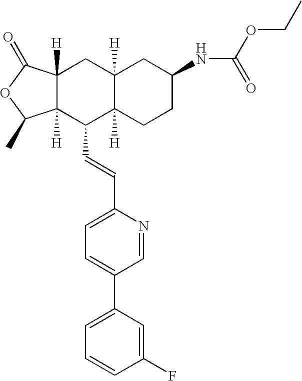
or a pharmaceutically acceptable salt, solvate, or ester of said compound.

2. A compound of the formula: 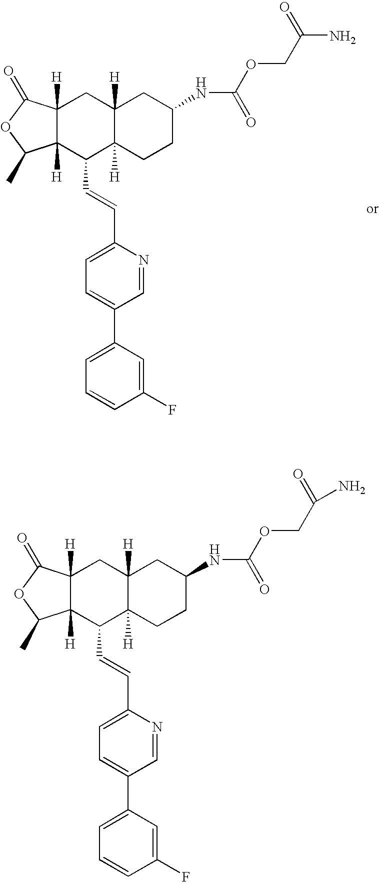
or a pharmaceutically acceptable salt, solvate, or ester thereof.

3. A compound of the formula: 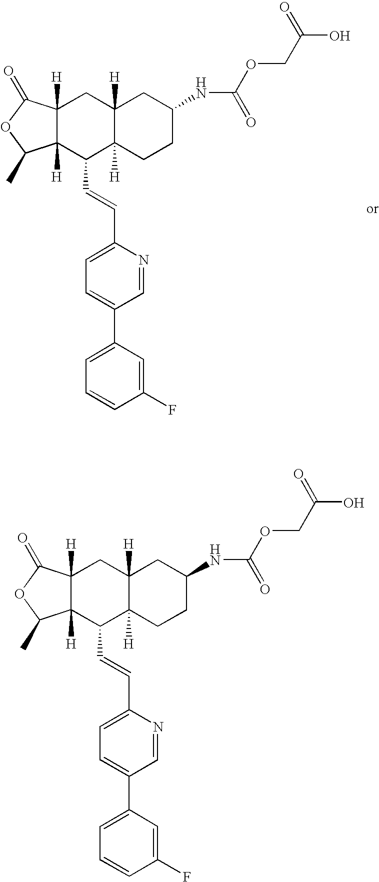
or a pharmaceutically acceptable salt, solvate, or ester thereof.

4. A compound of the formula: 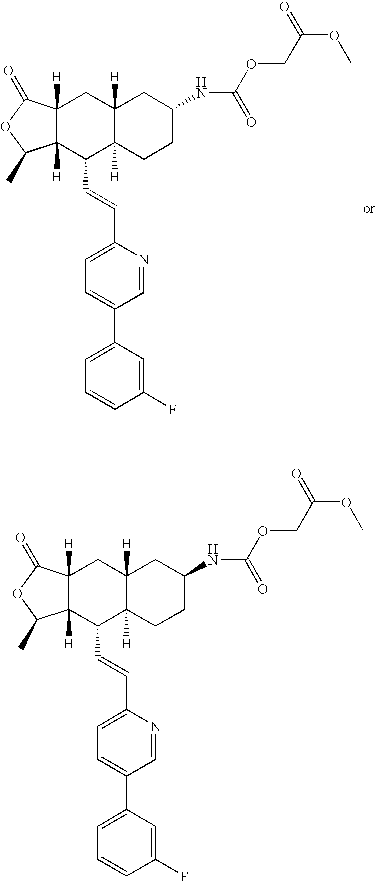
or a pharmaceutically acceptable salt, solvate, or ester thereof.

5. A compound of the formula: 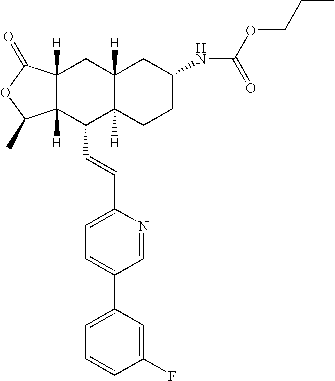
or a pharmaceutically acceptable salt, solvate, or ester thereof.

6. A compound of the formula: 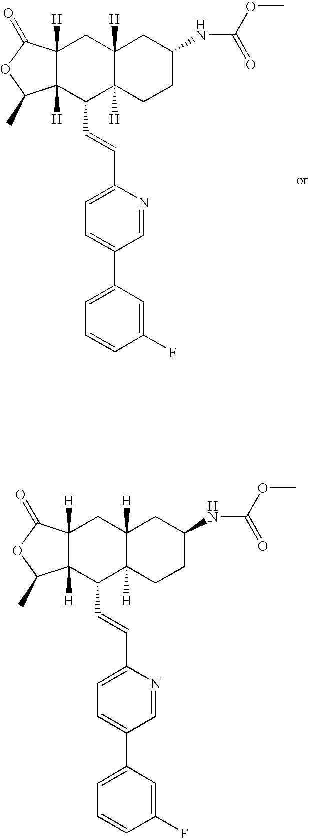
or a pharmaceutically acceptable salt, solvate, or ester thereof.

7. A compound of the formula: 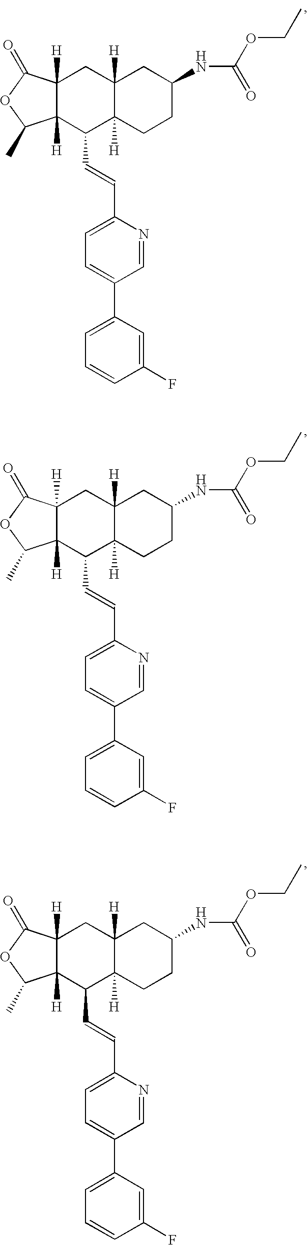 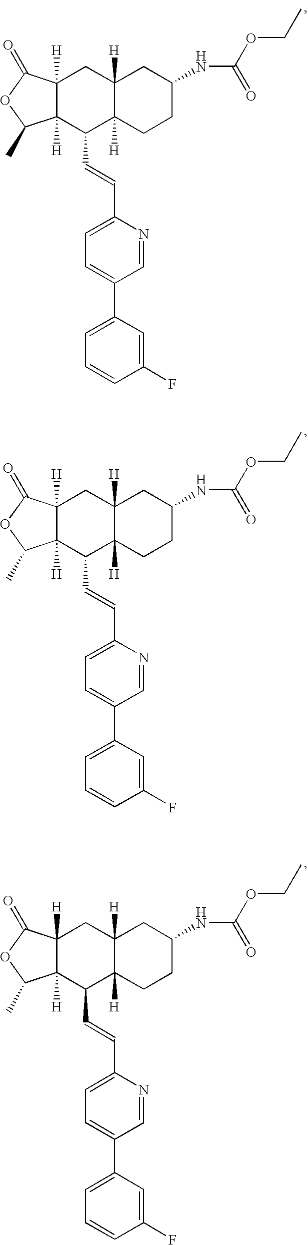 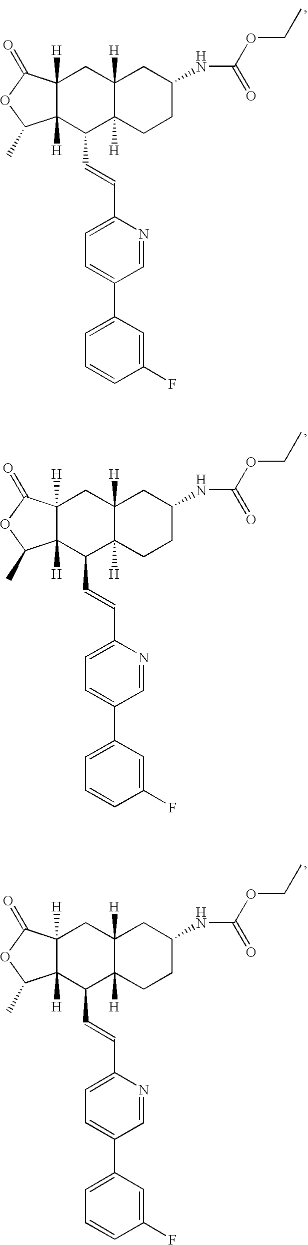 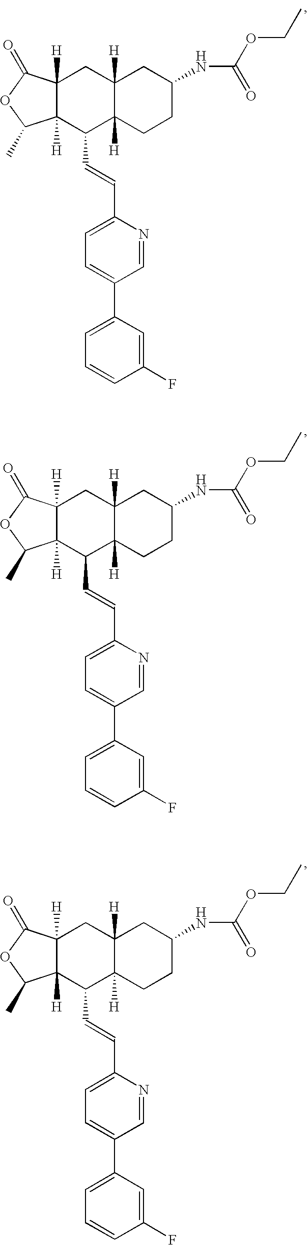 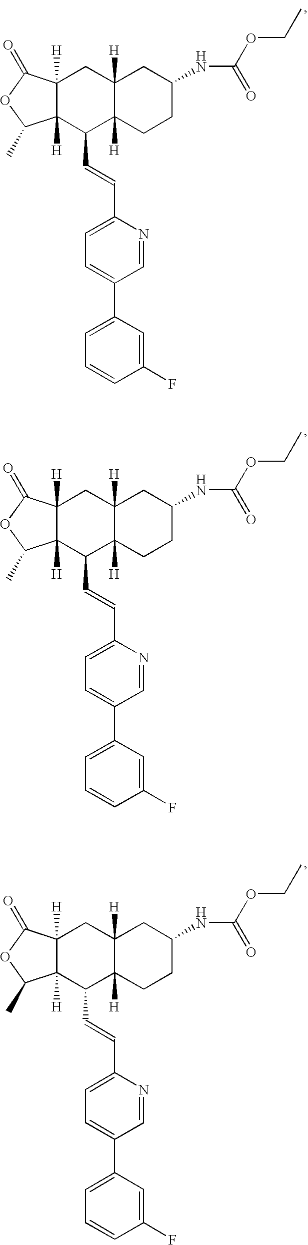 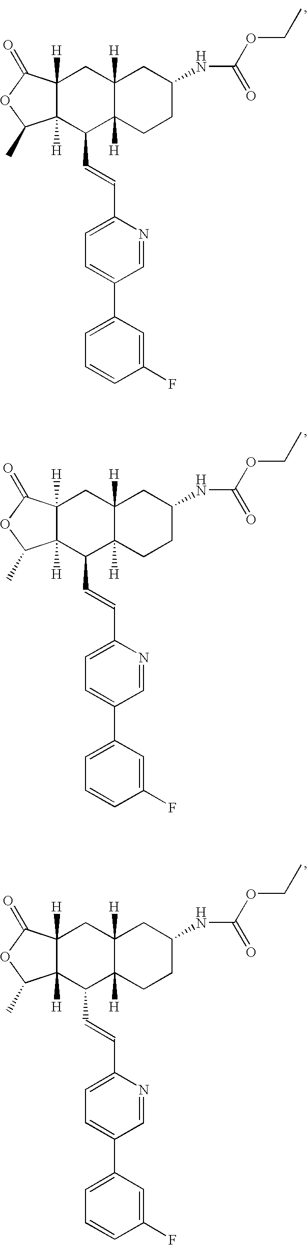 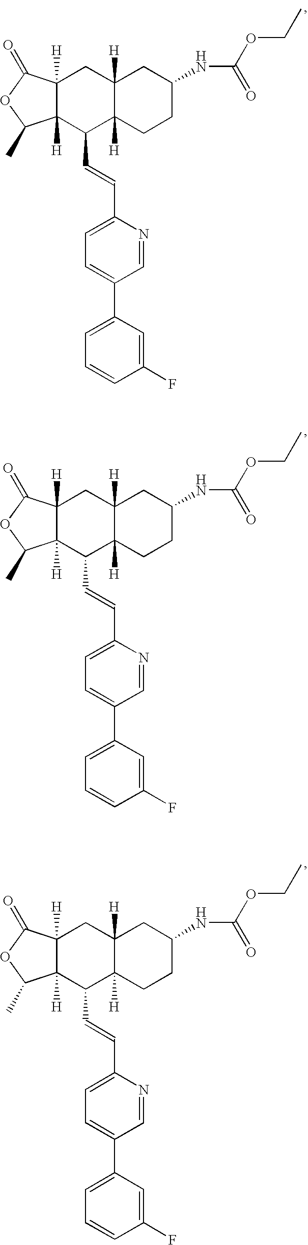 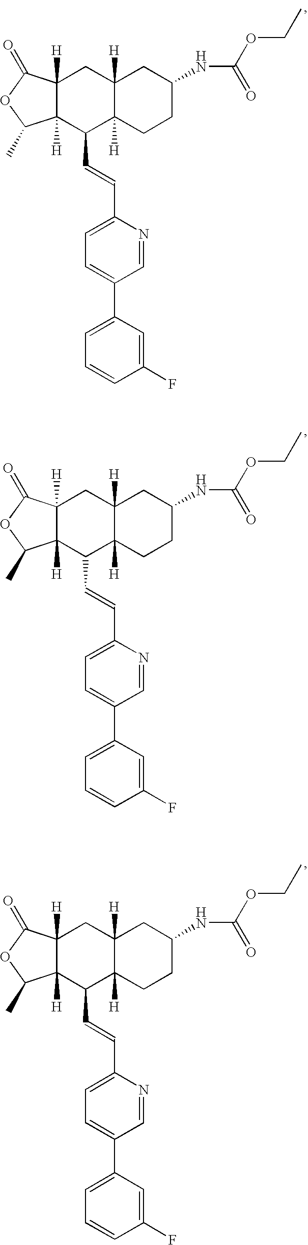 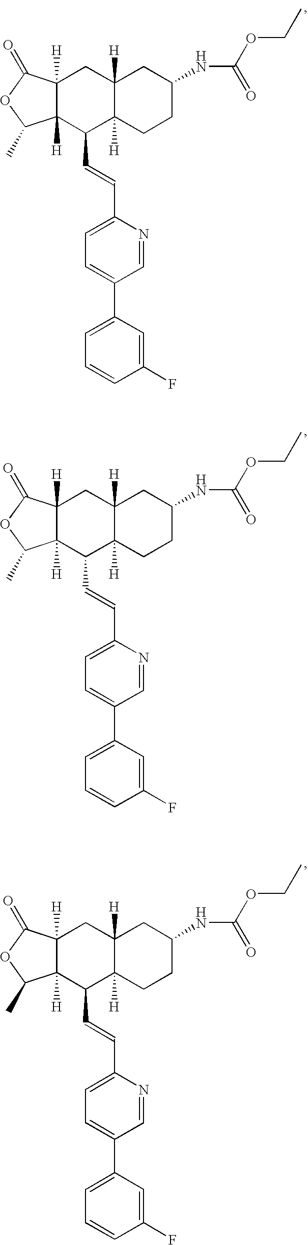 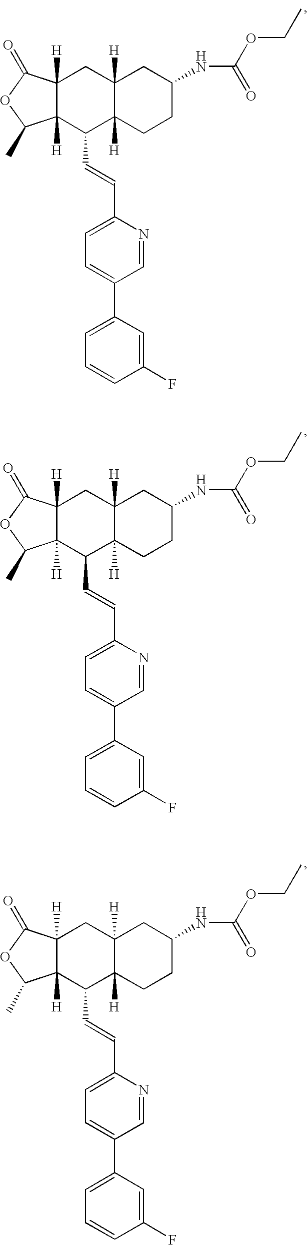 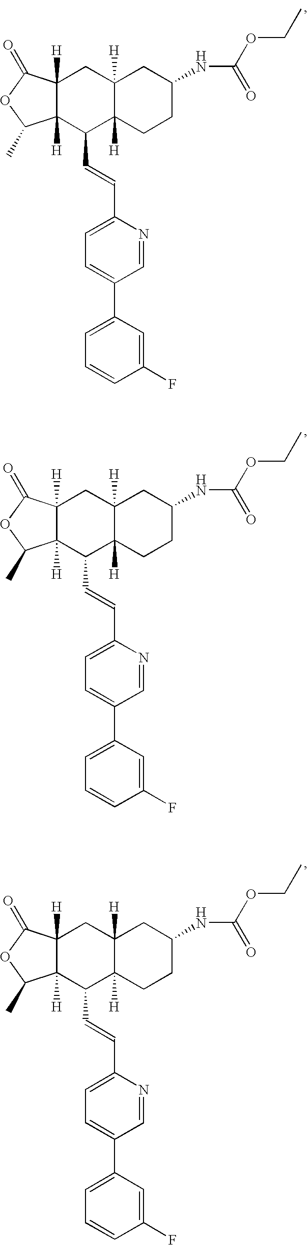 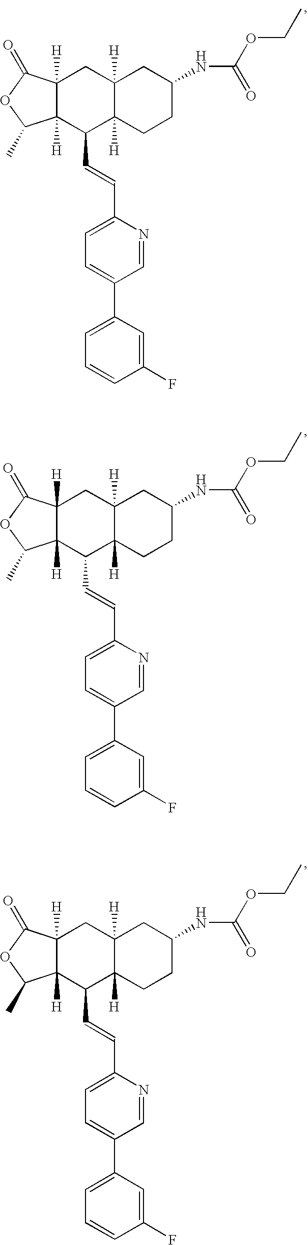 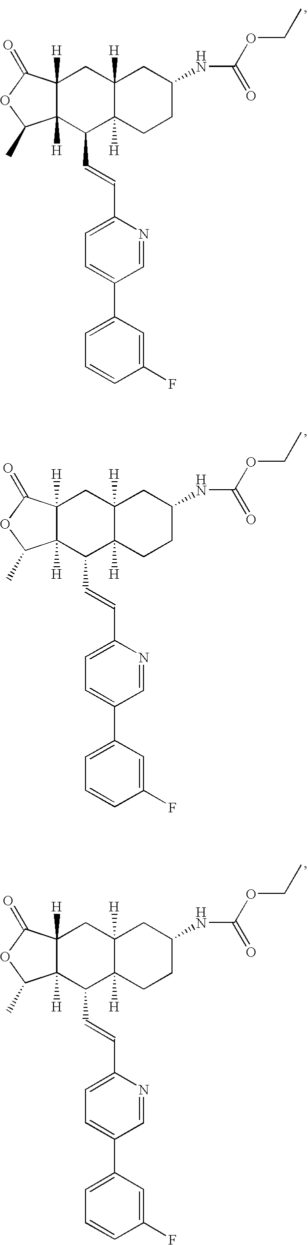 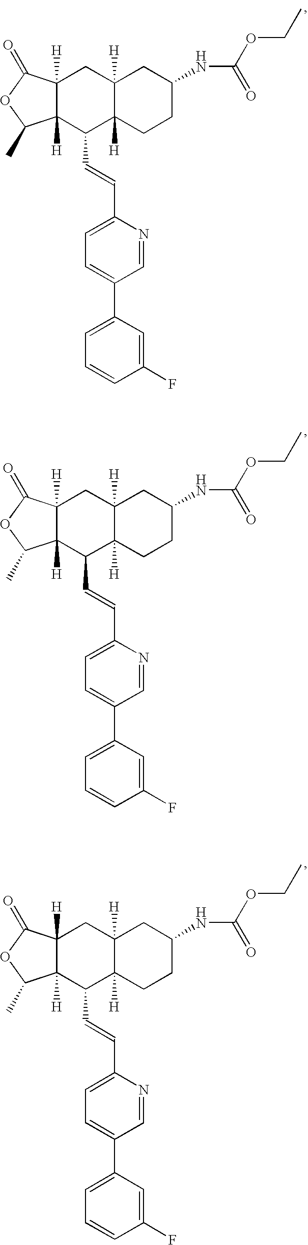 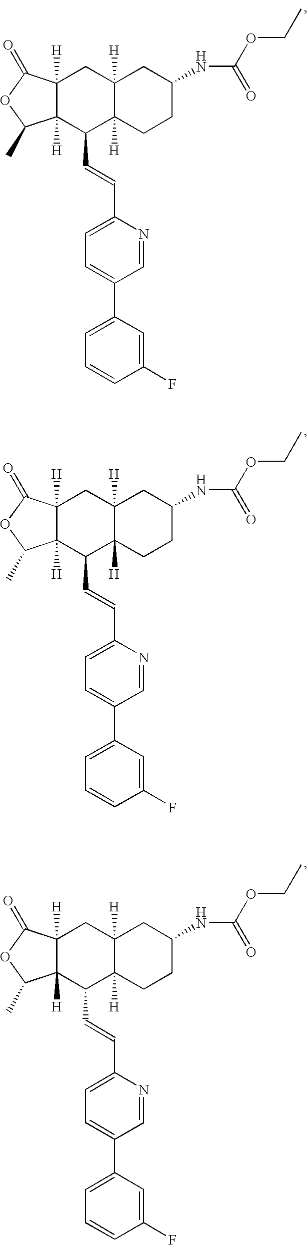 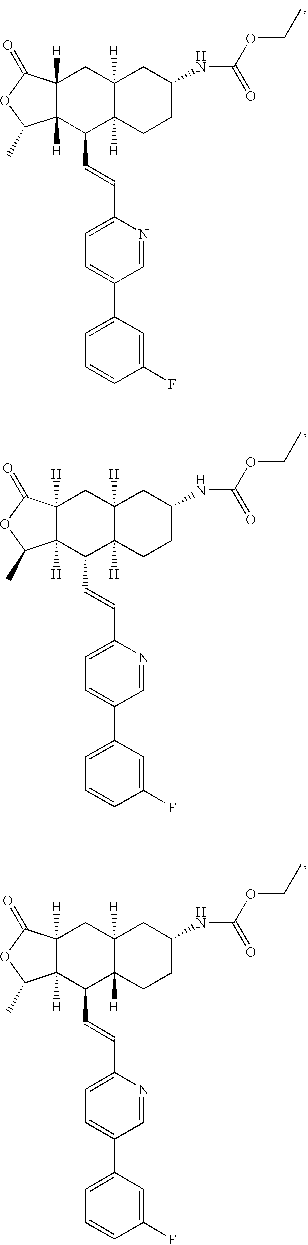 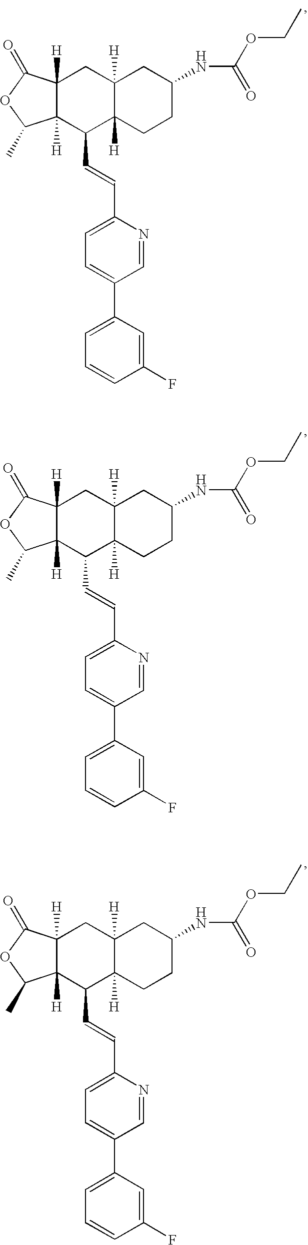 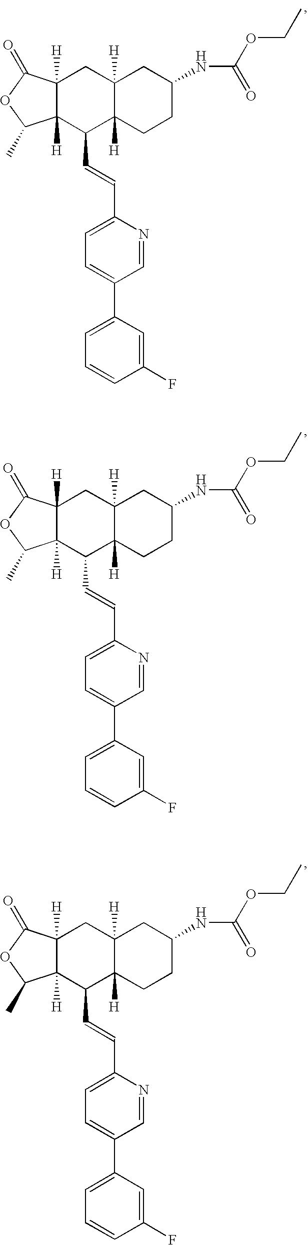 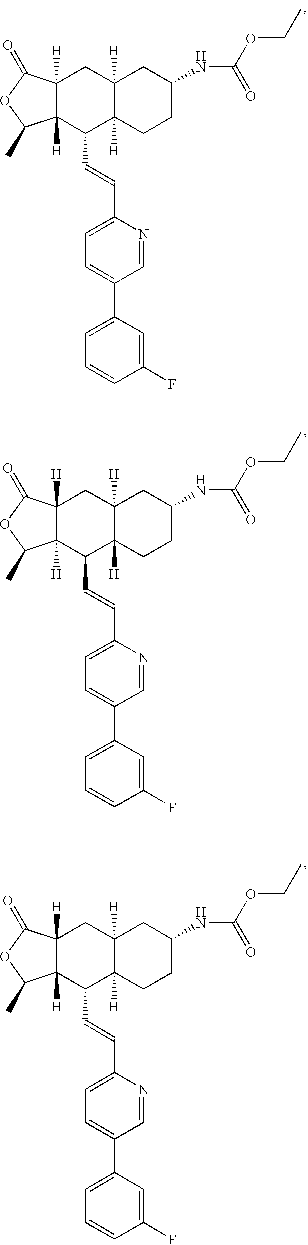 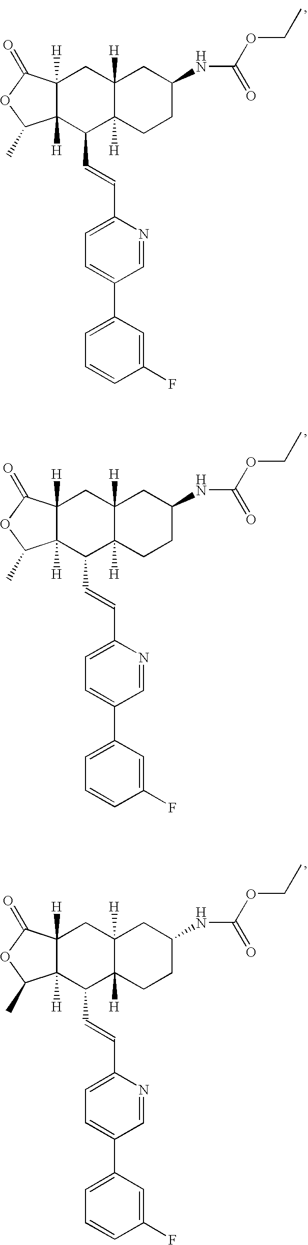 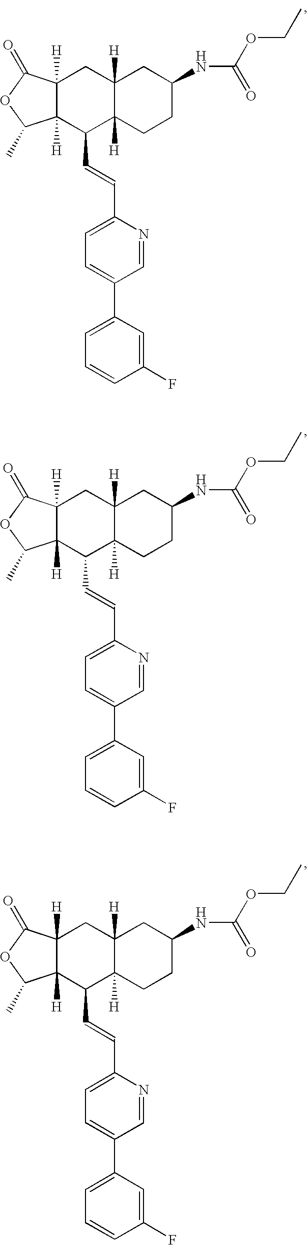 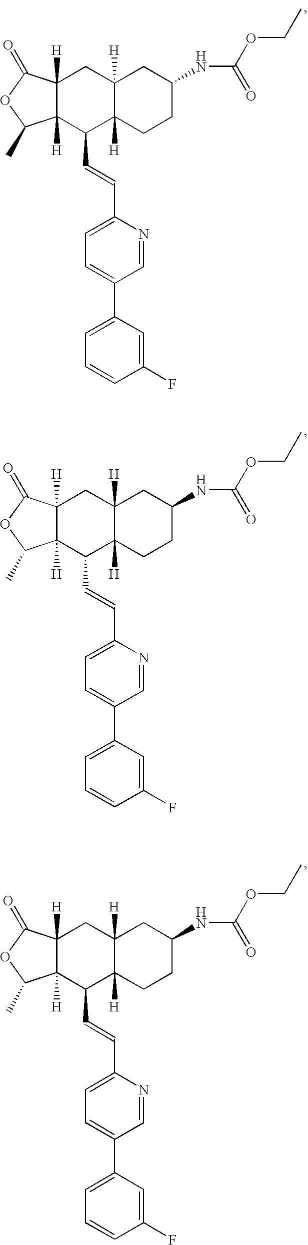 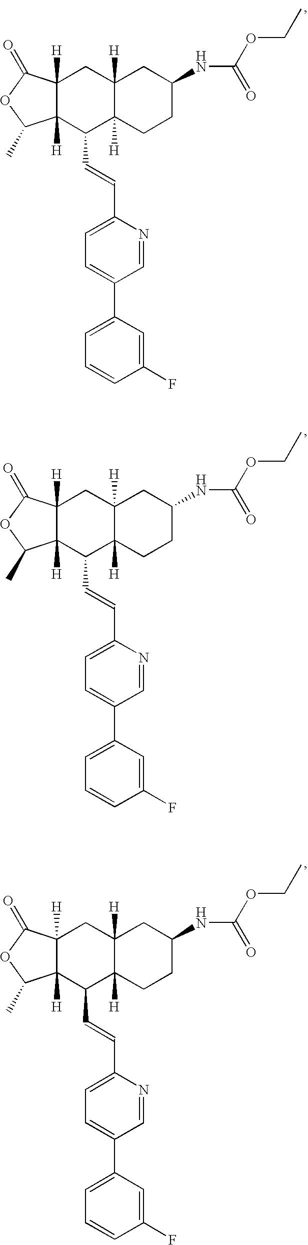 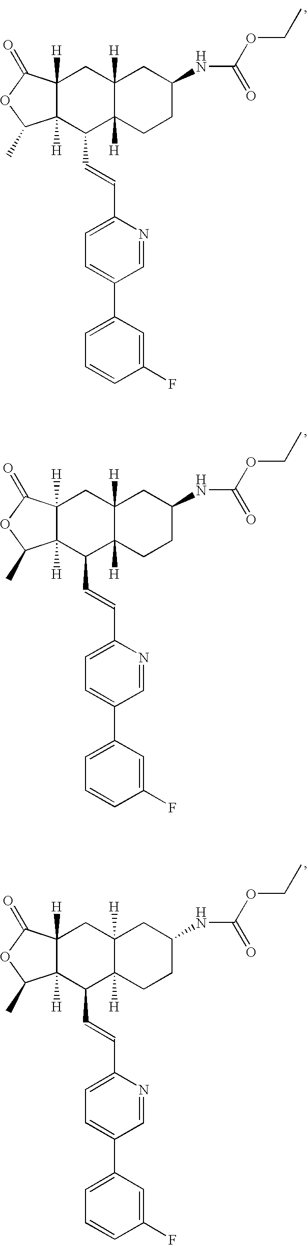 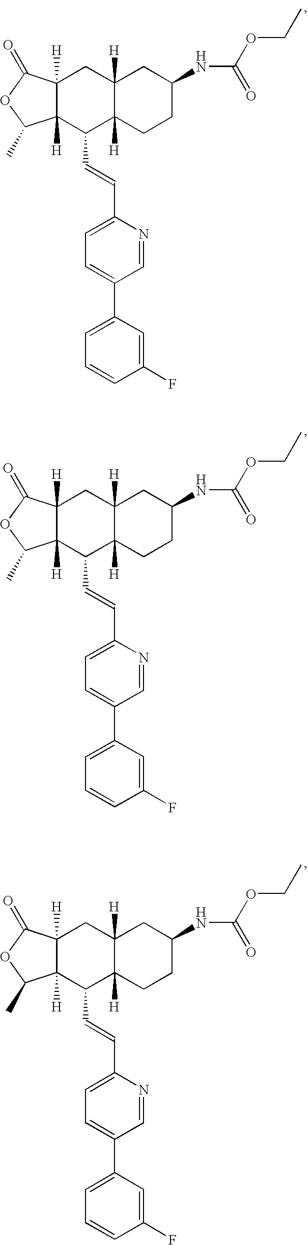 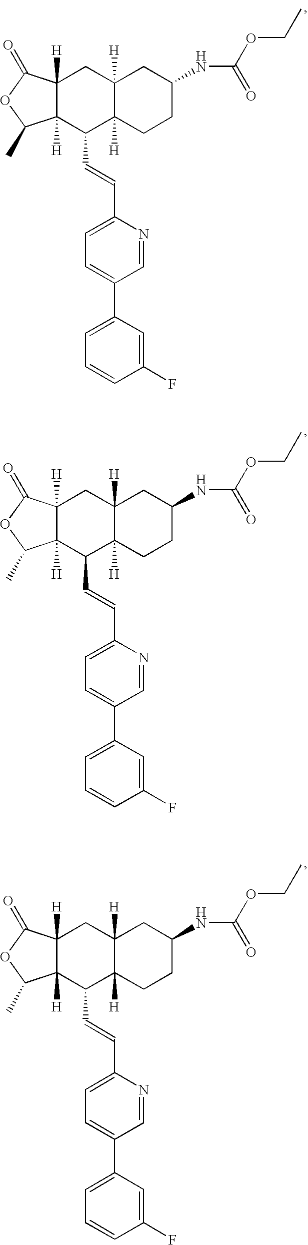 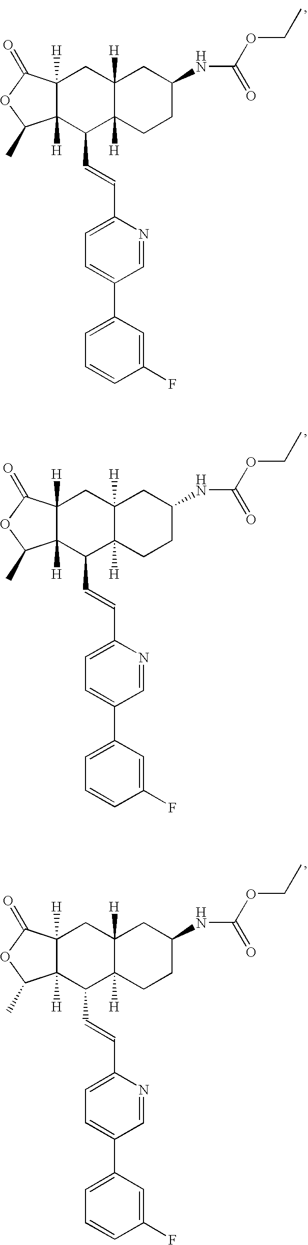 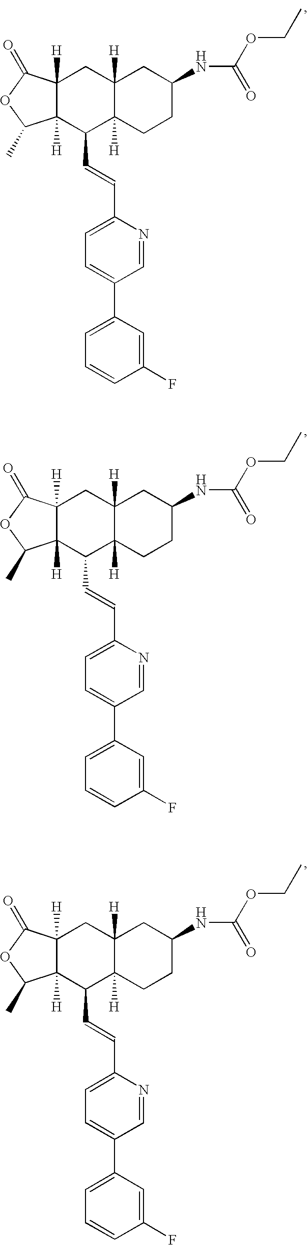 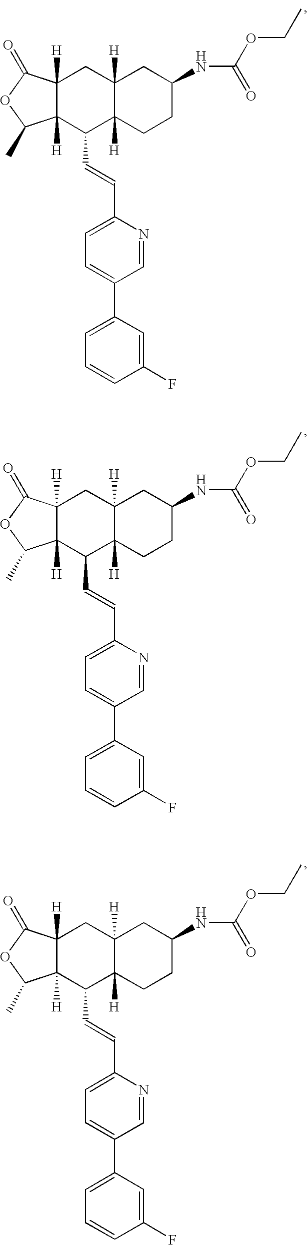 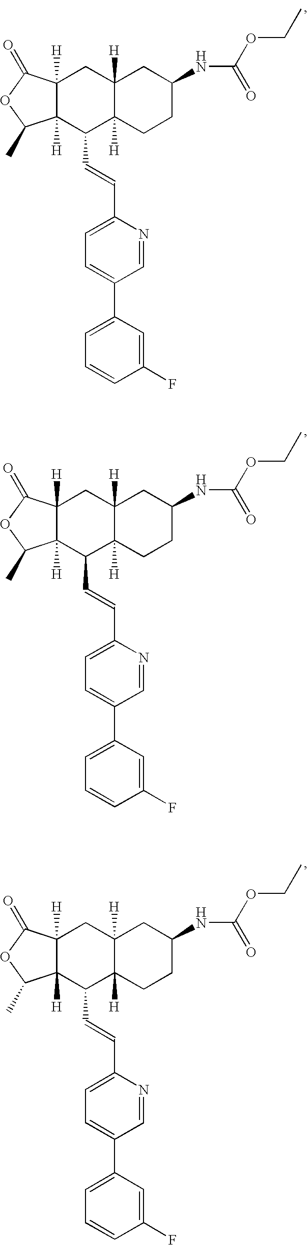 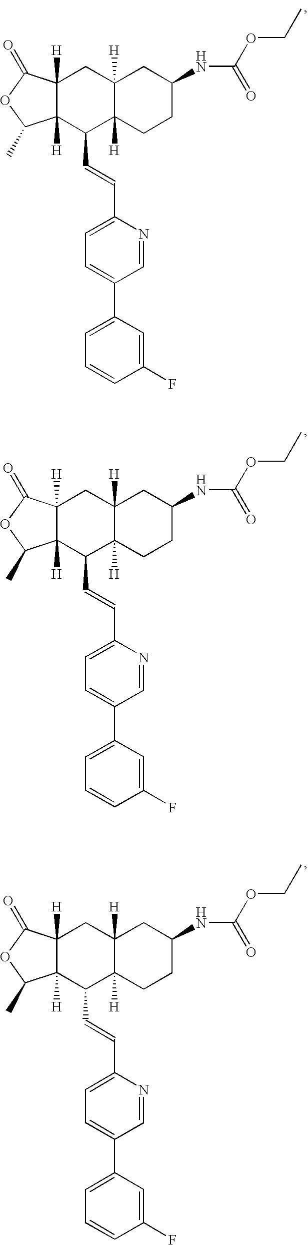 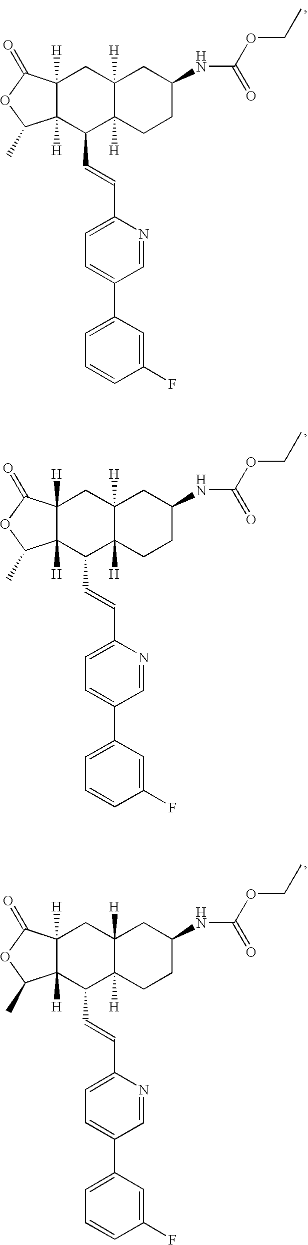 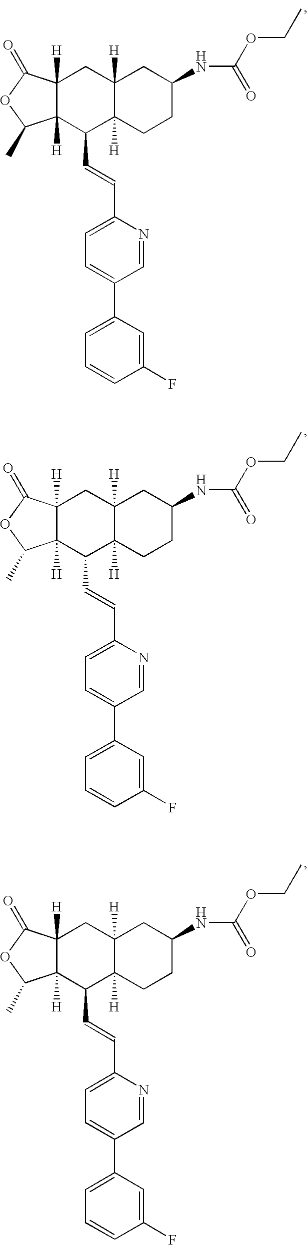 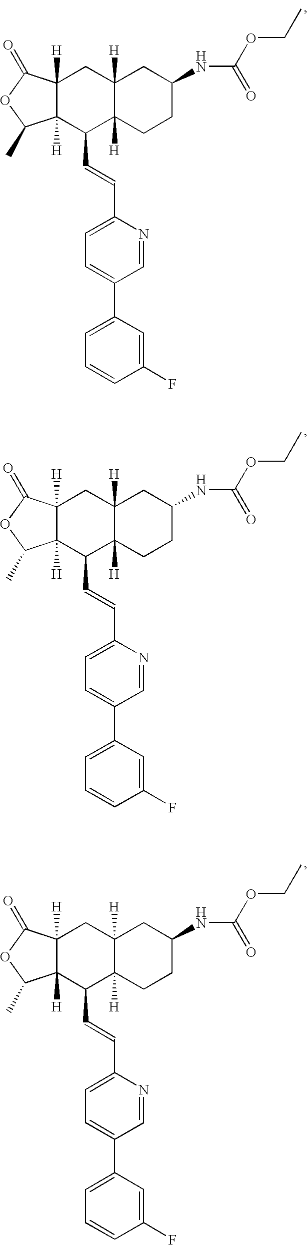 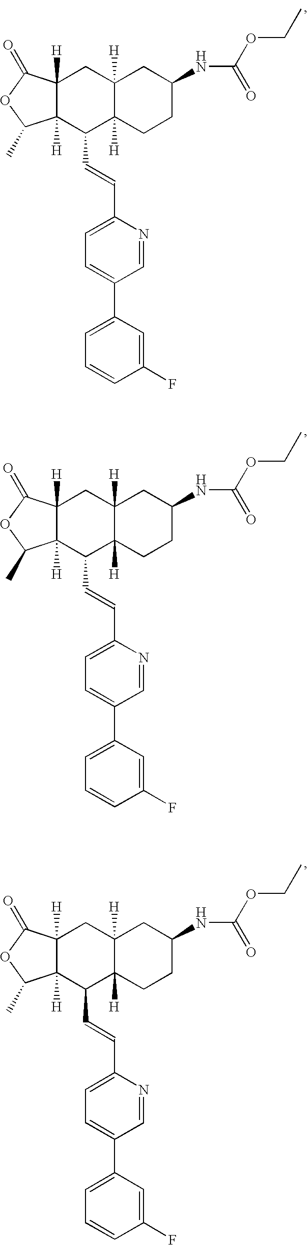 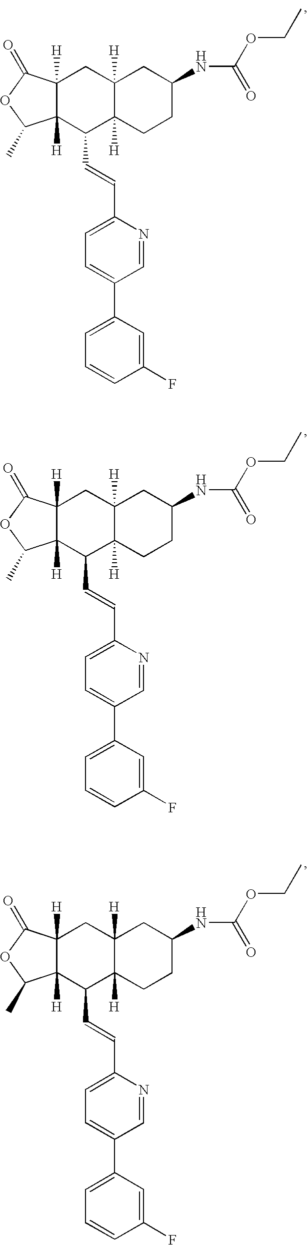 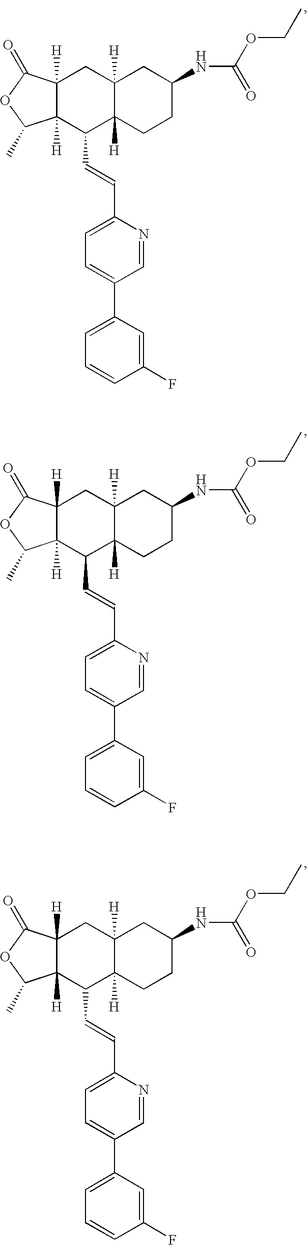 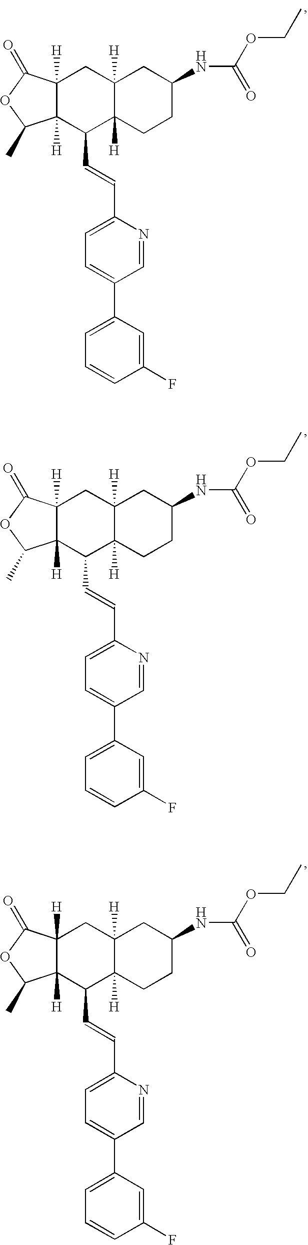 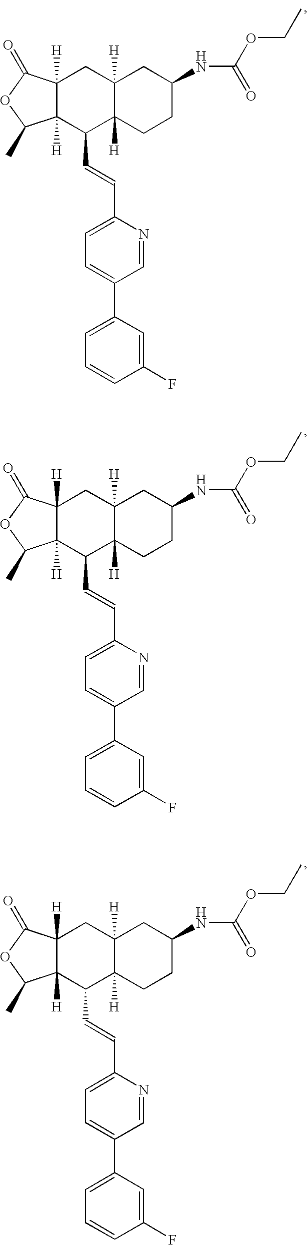 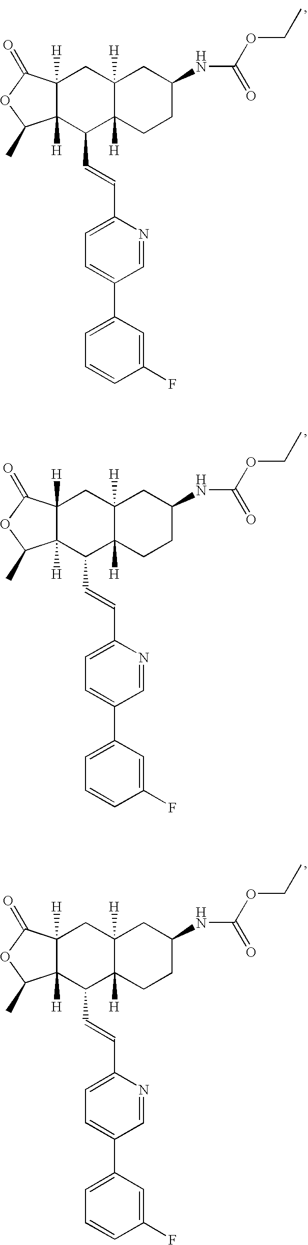 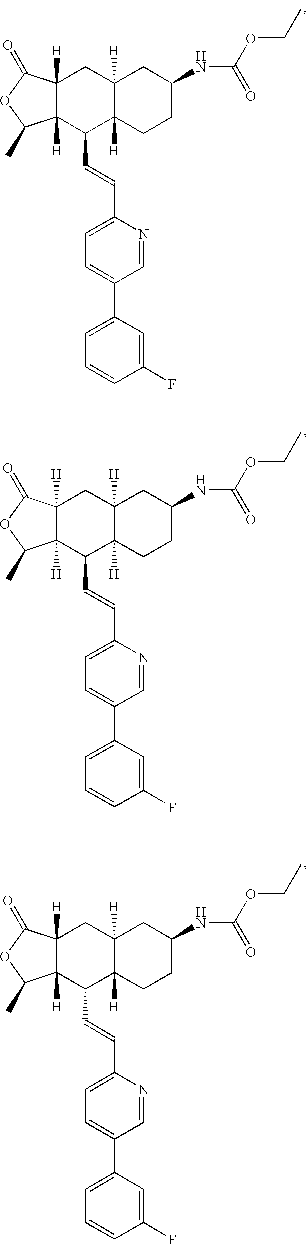 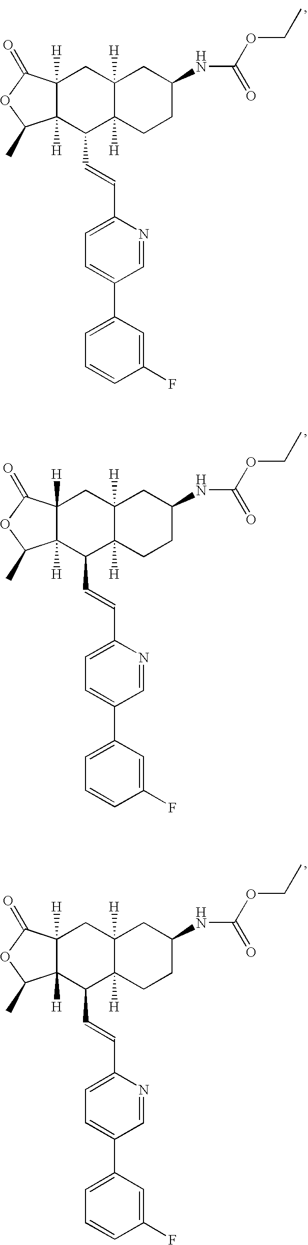 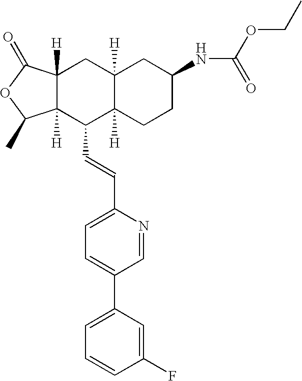
or a pharmaceutically acceptable salt, solvate, or ester thereof.

8. A pharmaceutical composition comprising an effective amount of at least one compound of claim 1 and a pharmaceutically acceptable carrier.

9. The pharmaceutical composition of claim 8, further comprising at least one additional cardiovascular agent to treat thrombosis, atherosclerosis, restenosis, hypertension, angina pectoris, angiogenesis related disorders, arrhythmia, a cardiovascular or circulatory disease or condition, heart failure, myocardial infarction, glomerulonephritis, thrombotic stroke, thromboembolytic stroke, peripheral vascular diseases, cerebral ischemia, rheumatoid arthritis, rheumatism, astrogliosis, a fibrotic disorder of the liver, kidney, lung or intestinal tract, systemic lupus erythematosus, multiple sclerosis, osteoporosis, glomerulonephritis, renal disease, acute renal failure, chronic renal failure, renal vascular homeostasis, renal ischemia, bladder inflammation, diabetes, diabetic neuropathy, cerebral stroke, cerebral ischemia, nephritis, cancer, melanoma, renal cell carcinoma, neuropathy and/or malignant tumors, neurodegenerative and/or neurotoxic diseases, conditions, or injuries, inflammation, asthma, glaucoma, macular degeneration, psoriasis, endothelial dysfunction disorders of the liver, kidney or lung inflammatory disorders of the lungs and gastrointestinal tract, respiratory tract disease or condition, radiation fibrosis, endothelial dysfunction, periodontal diseases or wounds or a spinal cord injury, or a symptom or result thereof.

10. The pharmaceutical composition of claim 9 wherein the additional cardiovascular agent or agents is selected from the group consisting of thromboxane A2 biosynthesis inhibitors, GP IIb/IIIa antagonists, thromboxane antagonists, adenosine diphosphate inhibitors, cyclooxygenase inhibitors, angiotensin antagonists, endothelin antagonists, angiotensin converting enzyme inhibitors, neutral endopeptidase inhibitors, anticoagulants, diuretics, and platelet aggregation inhibitors.

11. The pharmaceutical composition of claim 9 wherein the additional cardiovascular agent or agents are aspirin, cangrelor, clopidogrel bisulfate, parsugrel and fragmin.

12. The pharmaceutical composition of claim 9 wherein the additional cardiovascular agents are aspirin and clopidogrel bisulfate.

13. A method of inhibiting thrombin receptors comprising administering to a mammal in need of such treatment an effective amount of at least one compound of claim 1.

14. A method of treating thrombosis, atherosclerosis, restenosis, hypertension, angina pectoris, angiogenesis related disorders, arrhythmia, a cardiovascular or circulatory disease or condition, heart failure, myocardial infarction, glomerulonephritis, thrombotic stroke, thromboembolytic stroke, peripheral vascular diseases, cerebral ischemia, rheumatoid arthritis, rheumatism, astrogliosis, a fibrotic disorder of the liver, kidney, lung or intestinal tract, systemic lupus erythematosus, multiple sclerosis, osteoporosis, glomerulonephritis, renal disease, acute renal failure, chronic renal failure, renal vascular homeostasis, renal ischemia, bladder inflammation, diabetes, diabetic neuropathy, cerebral stroke, cerebral ischemia, nephritis, cancer, melanoma, renal cell carcinoma, neuropathy and/or malignant tumors, neurodegenerative and/or neurotoxic diseases, conditions, or injuries, inflammation, asthma, glaucoma, macular degeneration, psoriasis, endothelial dysfunction disorders of the liver, kidney or lung inflammatory disorders of the lungs and gastrointestinal tract, respiratory tract disease or condition, radiation fibrosis, endothelial dysfunction, periodontal diseases or wounds or a spinal cord injury, or a symptom or result thereof, comprising administering to a mammal in need of such treatment an effective amount of at least one compound of claim 1.

15. The method of claim 14 wherein the inflammatory disease or condition is irritable bowel syndrome, Crohn's disease, nephritis or a radiation- or chemotherapy-induced proliferate or inflammatory disorder of the gastrointestinal tract, lung, urinary bladder, gastrointestinal tract or other organ.

16. The method of claim 14 wherein the respiratory tract disease or condition is reversible airway obstruction, asthma, chronic asthma, bronchitis or chronic airways disease.

17. The method of claim 14 wherein the cancer is renal cell carcinoma or an angiogenesis related disorder.

18. The method of claim 14 wherein the neurodegenerative disease is Parkinson's disease, Amy tropic lateral sclerosis, Alzheimer's disease, Huntington's disease or Wilson's disease.

19. A method of treating thrombosis, atherosclerosis, restenosis, hypertension, angina pectoris, angiogenesis related disorders, arrhythmia, a cardiovascular or circulatory disease or condition, heart failure, myocardial infarction, glomerulonephritis, thrombotic stroke, thromboembolytic stroke, peripheral vascular diseases, cerebral ischemia, rheumatoid arthritis, rheumatism, astrogliosis, a fibrotic disorder of the liver, kidney, lung or intestinal tract, systemic lupus erythematosus, multiple sclerosis, osteoporosis, glomerulonephritis, renal disease, acute renal failure, chronic renal failure, renal vascular homeostasis, renal ischemia, bladder inflammation, diabetes, diabetic neuropathy, cerebral stroke, cerebral ischemia, nephritis, cancer, melanoma, renal cell carcinoma, neuropathy and/or malignant tumors, neurodegenerative and/or neurotoxic diseases, conditions, or injuries, inflammation, asthma, glaucoma, macular degeneration, psoriasis, endothelial dysfunction disorders of the liver, kidney or lung inflammatory disorders of the lungs and gastrointestinal tract, respiratory tract disease or condition, radiation fibrosis, endothelial dysfunction, periodontal diseases or wounds or a spinal cord injury, or a symptom or result thereof, comprising administering to a mammal in need of such treatment an effective amount of a compound of claim 1 in combination with at least one additional cardiovascular agent.

20. The method of claim 19 wherein the additional cardiovascular agent or agents is selected from the group consisting of thromboxane A2 biosynthesis inhibitors, GP IIb/IIIa antagonists, thromboxane antagonists, adenosine diphosphate inhibitors, cyclooxygenase inhibitors, angiotensin antagonists, endothelin antagonists, angiotensin converting enzyme inhibitors, neutral endopeptidase inhibitors, anticoagulants, diuretics, and platelet aggregation inhibitors.

21. The method of claim 19 wherein the additional cardiovascular agent or agents are aspirin, cangrelor, clopidogrel bisulfate, parsugrel and fragmin.

22. The method of claim 19 wherein the additional cardiovascular agents are aspirin and clopidogrel bisulfate.

23. A method of inhibiting cannabinoid receptors comprising administering to a mammal in need of such treatment an effective amount of at least one compound of claim 1.

24. A compound of claim 1 in purified form.

25. A compound of claim 1 in isolated form.

26. A method of treating or preventing radiation- or chemical-induced toxicity in non-malignant tissue in a patient comprising administering a therapeutically effective amount of at least one compound of claim 1.

27. The method of claim 26 wherein the radiation- and/or chemical-induced toxicity is one or more of intestinal fibrosis, pneumonitis, intestinal mucositis, oral mucositis, intestinal radiation syndrome, or pathophysiological manifestations of intestinal radiation exposure.

28. A method of reducing structural radiation injury in a patient that will be exposed, is concurrently exposed, or was exposed to radiation and/or chemical toxicity; reducing inflammation in a patient that will be exposed, is concurrently exposed, or was exposed to radiation and/or chemical toxicity; adverse tissue remodeling in a patient that will be exposed, is concurrently exposed, or was exposed to radiation and/or chemical toxicity- or reducing fibroproliferative tissue effects in a patient that will be exposed, is concurrently exposed, or was exposed to radiation and/or chemical toxicity, comprising administering a therapeutically effective amount of at least one compound of claim 1.

29. A method of treating a cell proliferative disorder in a patient suffering therefrom comprising administering a therapeutically effective amount of at least one compound of claim 1.

30. The method of claim 29 wherein the cell proliferative disorder is pancreatic cancer, glioma, ovarian cancer, colorectal cancer, colon cancer, breast cancer, prostate cancer, thyroid cancer, lung cancer, melanoma, or stomach cancer.

31. The method of claim 30 wherein the glioma is an anaplastic astrocytoma or a glioblastoma multiforme.
